(19) United States
(12) Patent Application Publication
(10) Pub. No.: US 2026/0207864 A1
(43) Pub. Date: Jul. 23, 2026

(54) DRIVER AND MIST INHALATION POD

(71) Applicant: SHAHEEN INNOVATIONS HOLDING LIMITED, Abu Dhabi (AE)

(72) Inventor: Mohammed Alshaiba Saleh Ghannam Almazrouei, Abu Dhabi (AE)

(21) Appl. No.: 19/550,181

(22) Filed: Feb. 25, 2026

Related U.S. Application Data
(63) Continuation-in-part of application No. 19/530,070, filed on Feb. 4, 2026.
(63) Continuation-in-part of application No. 19/255,014, filed on Jun. 30, 2025.
(63) Continuation-in-part of application No. 18/971,899, filed on Dec. 6, 2024.
(60) Provisional application No. 63/606,859, filed on Dec. 6, 2023.
(60) Provisional application No. 63/679,371, filed on Aug. 5, 2024.
(60) Provisional application No. 63/928,619, filed on Dec. 1, 2025.

(51) Int. Cl. A61M 15/00; A24F 40/10

(52) U.S. Cl. CPC A61M 15/001; A24F 40/10; A61M 15/0021; A61M 15/0085; A61M 15/0095; A61M 2205/3334; A61M 2205/587

(57) ABSTRACT

A mist inhaler including a pod and a driver, the pod comprising: a housing having a first end, an opposite second end and at least one side wall extending therebetween;
a liquid chamber containing a liquid comprising nicotine; a spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel and a cavity, the cavity including a first aperture for allowing fluid flow in a first direction, and one or more further apertures for allowing fluid flow in a second direction; a sonication chamber including the cavity of the fluid flow manifold; an ultrasonic transducer; an air inlet conduit; and
a mist outlet conduit; the driver comprising: a driver casing having an air intake aperture; a casing recess; electronic components configured to generate control signals to control the operation of the pod, and drive signals to drive the pod to generate a mist; and a bulkhead positioned within the casing recess, the bulkhead including: a body including a portion that extends from the top surface to form a seal; a plurality of apertures formed in the body and positioned in an area at least partly surrounded by the seal; and a bulkhead intake conduit in airtight fluid communication with the air intake aperture and with a first aperture of the plurality of apertures, the bulkhead intake conduit being configured to conduct air from the air intake aperture to the pod through the first aperture of the plurality of apertures in the bulkhead; wherein, when the driver is coupled to the pod, a part of the pod is received into the casing recess, the seal of the driver contacting the part of the pod forming a substantially airtight seal therebetween to prevent air leaking out from between the bulkhead and the part of the pod.

### CROSS REFERENCES TO RELATED APPLICATIONS

The present application is a continuation-in-part of U.S. application Ser. No. 19/530,070, filed 4 Feb. 2026 which claims the benefit of priority to U.S. provisional application No. 63/928,619, filed 1 Dec. 2025. The present application is also a continuation-in-part of U.S. application Ser. No. 19/255,014, filed 30 Jun. 2025, which claims the benefit of priority to U.S. provisional application No. 63/679,371, filed 5 Aug. 2024. The present application is also a continuation-in-part of U.S. application Ser. No. 18/971,899, filed 6 Dec. 2024, which claims the benefit of priority to U.S. provisional application No. 63/606,859, filed 6 Dec. 2023. All of the foregoing applications are incorporated herein by reference in their entirety.

### FIELD

The present invention relates to a mist inhaler including a driver and a mist inhalation pod.

### BACKGROUND

Mist inhalers are used for generating a mist or vapour for inhalation by a user. The mist may contain a drug or medicine which is inhaled by a user and absorbed into the user's blood stream.

In particular, mist inhalers or electronic vaporising inhalers are becoming popular among smokers who want to avoid the tar and other harsh chemicals associated with traditional cigarettes and who wish to satisfy the craving for nicotine. Electronic vaporising inhalers may contain liquid nicotine, which is typically a mixture of nicotine oil, a solvent, water, and often flavouring. When the user draws, or inhales, on the electronic vaporising inhaler, the liquid nicotine is drawn into a vaporiser where it is heated into a vapour. As the user draws on the electronic vaporising inhaler, the vapour containing the nicotine is inhaled. Such electronic vaporising inhalers may have medical purpose.

Electronic vaporising inhalers and other vapour inhalers typically have similar designs. Most electronic vaporising inhalers feature a liquid nicotine reservoir with an interior membrane, such as a capillary element, typically cotton, that holds the liquid nicotine so as to prevent leaking from the reservoir. Nevertheless, these cigarettes are still prone to leaking because there is no obstacle to prevent the liquid from flowing out of the membrane and into the mouthpiece. A leaking electronic vaporising inhaler is problematic for several reasons. As a first disadvantage, the liquid can leak into the electronic components, which can cause serious damage to the device. As a second disadvantage, the liquid can leak into the electronic vaporising inhaler mouthpiece, and the user may inhale the unvapourised liquid.

Electronic vaporising inhalers are also known for providing inconsistent doses between draws. The aforementioned leaking is one cause of inconsistent doses because the membrane may be oversaturated or undersaturated near the vaporiser. If the membrane is oversaturated, then the user may experience a stronger than desired dose of vapour, and if the membrane is undersaturated, then the user may experience a weaker than desired dose of vapour. Additionally, small changes in the strength of the user's draw may provide stronger or weaker doses. Inconsistent dosing, along with leaking, can lead to faster consumption of the vaping liquid.

Additionally, conventional electronic vaporising inhalers tend to rely on inducing high temperatures of a metal heating component configured to heat a liquid in the e-cigarette, thus vaporising the liquid that can be breathed in. Problems with conventional electronic vaporising inhalers may include the possibility of burning metal and subsequent breathing in of the metal along with the burnt liquid. In addition, some may not prefer the burnt smell caused by the heated liquid.

Thus, a need exists in the art for improved mist inhalers which seek to address at least some of the problems described herein.

### BRIEF DESCRIPTION OF THE INVENTION

The present invention provides a mist inhaler as claimed in claim 1 and claim 12. Preferred features of the invention are provided in the dependent claims.

The various examples of this disclosure are described below and have multiple advantages and benefits over conventional vaporising inhalers. These advantages and benefits are set out in the description below.

### REPRESENTATIVE FEATURES

Representative features are set out in the following clauses, which stand alone or may be combined, in any combination, with one or more features disclosed in the text and/or drawings of the specification.

According to an aspect of the present disclosure, there is provided a driver for use with a mist inhalation pod, the driver comprising: a driver casing having a base end, an open end, and an air intake aperture positioned between the base end and the open end, the open end incorporating a casing recess; electronic components configured to generate control signals to control the operation of the pod, and drive signals to drive the pod to generate a mist, the mist comprising nicotine; and a bulkhead positioned within the casing recess, the bulkhead including: a body substantially of a resiliently deformable material, the body having a top surface, a bottom surface and a portion that extends from the top surface to form a seal; a plurality of apertures formed in the body, each aperture extending through the top surface and the bottom surface, the plurality of apertures being positioned in an area at least partly surrounded by the seal; and a bulkhead intake conduit in airtight fluid communication with the air intake aperture and with a first aperture of the plurality of apertures, the bulkhead intake conduit being formed entirely within the driver and forming a part of an intake air flow path, the bulkhead intake conduit being configured to conduct air from the air intake aperture to the pod through the first aperture of the plurality of apertures in the bulkhead; wherein, when the driver is coupled to the pod, a part of the pod is received into the casing recess, the seal of the driver contacting the part of the pod forming a substantially airtight seal therebetween to prevent air leaking out from between the bulkhead and the part of the pod.

In some examples, the seal is positioned proximate to the perimeter of the bulkhead body.

In some examples, the seal is an upturned lip extending around the periphery of the top surface.

In some examples, the seal spaces the part of the pod from the top surface of the body of the bulkhead by a distance, the distance defining a chamber therebetween.

In some examples, the chamber defines a part of the intake air flow path.

In some examples, the driver further includes a plurality of electrical connectors, each of the electrical connectors extending through a respective one of the plurality of apertures formed in the body of the bulkhead, the body of the bulkhead providing an airtight seal around each of the electrical connectors such that air cannot flow from the top surface of the body to the bottom surface of the body.

In some examples, the driver further includes: an air flow sensor for detecting a change in air pressure relative to an ambient air pressure and providing an air flow pressure signal to the electronic components within the driver; an air flow sensor holder within the driver casing, the air flow sensor holder including an opening for accepting the air flow sensor, the opening having a perimeter contacting the air flow sensor to form an airtight seal with the air flow sensor to create an ambient air space and an intake air space separated by the air flow sensor, wherein the air flow sensor holder includes: a channel for supplying air at substantially the ambient air pressure to the ambient air space; and a further channel in fluid communication with the intake air flow path extending from the driver casing to the pod, the further channel for detecting an air pressure in the intake air flow path lower than the ambient air pressure, the lower air pressure being indicative of a user using the pod.

In some examples, the driver further comprises at least one LED, the air flow sensor holder including a recess for accepting a respective one of the at least one LEDs.

In some examples, the air flow sensor holder comprises: a body portion which includes the opening for accepting the air flow sensor, the channel for supplying air at substantially the ambient air pressure to the ambient air space, and the further channel for detecting an air pressure in the intake air flow path lower than the ambient air pressure; and an auxiliary portion extending from the base portion, the auxiliary portion covering each of the at least one LEDs and including a first side and a second side, the second side of the auxiliary portion including the recess for accepting a respective one of the at least one LEDs, the auxiliary portion further including at least one projection extending from and substantially normal to the first side of the auxiliary portion.

In some examples, the projection is translucent such that, when the at least one LED is activated, light is visible through the projection.

In some examples, the at least one LED is a plurality of LEDs, and wherein a corresponding projection is provided for each LED.

According to an aspect of the present disclosure, there is provided a bulkhead for use with a driver, the bulkhead including: a body substantially of a resiliently deformable material, the body having a top surface, a bottom surface and a portion that extends from the top surface to, in use, form a seal; a plurality of apertures formed in the body, each aperture extending through the top surface and the bottom surface, the plurality of apertures being positioned in an area at least partly surrounded by the seal; and a bulkhead intake conduit in airtight fluid communication with a first aperture of the plurality of apertures, the bulkhead intake conduit being configured to conduct air through the first aperture of the plurality of apertures in the bulkhead.

According to an aspect of the present disclosure, there is provided a driver for use with a mist inhalation pod, the driver comprising: a substrate; an air flow sensor coupled to the substrate for detecting a change in air pressure relative to the ambient air pressure; at least one LED coupled to the substrate; and an air flow sensor holder coupled to the substrate, the air flow sensor holder including: a body portion having an opening for accepting the air flow sensor, the opening having a perimeter contacting the air flow sensor to form an airtight seal with the air flow sensor to create an ambient air space and an intake air space separated by the air flow sensor, wherein the body portion of the air flow sensor holder includes: a channel for supplying air at substantially ambient air pressure to the ambient air space; a further channel in fluid communication with an intake air flow path extending from the driver casing to the pod, the further channel for detecting an air pressure in the intake air flow path lower than the ambient air pressure, the lower air pressure being indicative of a user using the pod; the air flow sensor holder having a recess for accepting a respective one of the at least one LEDs.

In some examples, the air flow sensor holder further comprises an auxiliary portion extending from the base portion, the auxiliary portion including a first side and a second side, the auxiliary portion covering each of the at least one LEDs and the second side of the auxiliary portion including the recess for accepting a respective one of the at least one LEDs, the auxiliary portion further including at least one projection extending from and substantially normal to the first side of the auxiliary portion.

In some examples, the projection is translucent such that, when the at least one LED is activated, light is visible through the projection.

In some examples, the substrate is a PCB.

In some examples, the driver includes a main PCB mounted therein, the substrate being the main PCB.

In some examples, the air flow sensor holder is formed of a single piece.

In some examples, the air flow sensor holder is of a resiliently deformable material.

In some examples, the air flow sensor holder is of Shore A 40 silicone.

In some examples, the at least one LED is a plurality of LEDs, and wherein a corresponding projection is provided for each LED.

According to an aspect of the present disclosure, there is provided an air flow sensor holder for use in a mist inhaler, the holder including: a body portion having an opening for accepting an air flow sensor, the opening having a perimeter for contacting the air flow sensor to form an airtight seal with the air flow sensor to create an ambient air space and an intake air space separated by the air flow sensor, wherein the body portion of the air flow sensor holder includes: a channel for supplying air at substantially ambient air pressure to the ambient air space; a further channel for detecting an air pressure lower than the ambient air pressure, the air flow sensor holder having a recess for accepting an LED.

According to an aspect of the present disclosure, there is provided a mist inhaler including a pod and a driver for delivering a mist including nicotine, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid chamber containing a liquid to be atomised, the liquid comprising the nicotine; a spacer positioned within the housing between the liquid chamber and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having a first portion and a second portion, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an ultrasonic transducer positioned between the sonication chamber and the second end wall, the ultrasonic transducer having an atomisation surface adjacent to the sonication chamber and in communication with the sonication chamber; an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and a mist outlet conduit, including the first aperture in the fluid flow manifold, forming an air-tight channel for conducting the mist through the liquid chamber and any liquid contained therein, the mist outlet conduit extending from the first end wall, through the liquid chamber, and through the fluid flow manifold to the sonication chamber, a first end of the mist outlet conduit being in fluid communication with a mist outlet port in the first end wall of the housing and a second end of the mist outlet conduit being in fluid communication with the sonication chamber, and the driver comprising: a driver casing having a base end, an open end, and an air intake aperture positioned between the base end and the open end, the open end incorporating a casing recess; electronic components configured to generate control signals to control the operation of the pod, and drive signals to drive the ultrasonic transducer to atomise the liquid to generate the mist; and a bulkhead positioned within the casing recess, the bulkhead including: a body substantially of a resiliently deformable material, the body having a top surface, a bottom surface and a portion that extends from the top surface to form a seal; a plurality of apertures formed in the body, each aperture extending through the top surface and the bottom surface, the plurality of apertures being positioned in an area at least partly surrounded by the seal; and a bulkhead intake conduit in airtight fluid communication with the air intake aperture and with a first aperture of the plurality of apertures, the bulkhead intake conduit being formed entirely within the driver and forming a part of an intake air flow path, the bulkhead intake conduit being configured to conduct air from the air intake aperture to the pod through the first aperture of the plurality of apertures in the bulkhead, a part of the pod being received into the casing recess, the seal of the driver contacting the part of the pod and forming a substantially airtight seal therebetween to prevent air leaking out from between the bulkhead and the part of the pod, and an intake air flow path extending from the air intake aperture in the driver casing to the sonication chamber, the intake air flow path passing through the bulkhead intake conduit in the driver, and the air intake conduit in the pod.

In some examples, the first portion of the channel in the fluid flow manifold extends to the second portion tangentially.

In some examples, the seal is an upturned lip extending around the periphery of the top surface.

In some examples, the seal spaces the part of the pod from the top surface of the body of the bulkhead by a distance, the distance defining a chamber therebetween.

In some examples, the chamber defines a part of the intake air flow path.

In some examples, the mist inhaler further includes a plurality of electrical connectors, each of the electrical connectors extending through a respective one of the plurality of apertures formed in the body of the bulkhead, the body of the bulkhead providing an airtight seal around each of the electrical connectors such that air cannot flow from the top surface of the body to the bottom surface of the body.

In some examples, the mist inhaler further includes: an air flow sensor for detecting a change in air pressure relative to an ambient air pressure and providing an air flow pressure signal to the electronic components within the driver; an air flow sensor holder within the driver casing, the air flow sensor holder including an opening for accepting the air flow sensor, the opening having a perimeter contacting the air flow sensor to form an airtight seal with the air flow sensor to create an ambient air space and an intake air space separated by the air flow sensor, wherein the air flow sensor holder includes: channel for supplying air at substantially the ambient air pressure to the ambient air space; and a further channel in fluid communication with the intake air flow path extending from the driver casing to the pod, the further channel for detecting an air pressure in the intake air flow path lower than the ambient air pressure, the lower air pressure being indicative of a user using the pod.

In some examples, the mist inhaler further comprises at least one LED, the air flow sensor holder including a recess for accepting a respective one of the at least one LEDs.

In some examples, the air flow sensor holder comprises: a body portion which includes the opening for accepting the air flow sensor, the channel for supplying air at substantially the ambient air pressure to the ambient air space, and the further channel for detecting an air pressure in the intake air flow path lower than the ambient air pressure; and an auxiliary portion extending from the base portion, the auxiliary portion covering each of the at least one LEDs and including a first side and a second side, the second side of the auxiliary portion including the recess for accepting a respective one of the at least one LEDs, the auxiliary portion further including at least one projection extending from and substantially normal to the first side of the auxiliary portion.

In some examples, the projection is translucent such that, when the at least one LED is activated, light is visible through the projection.

In some examples, the at least one LED is a plurality of LEDs, and wherein a corresponding projection is provided for each LED.

According to an aspect of the present disclosure, there is provided a mist inhaler including a pod and a driver for delivering a mist including nicotine, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid chamber, containing a liquid to be atomised, the liquid comprising the nicotine; a spacer positioned within the housing between the liquid chamber and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having a first portion and a second portion, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an ultrasonic transducer positioned between the sonication chamber and the second end wall, the ultrasonic transducer having an atomisation surface adjacent to the sonication chamber and in communication with the sonication chamber; an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and a mist outlet conduit, including the first aperture in the fluid flow manifold, forming an air-tight channel for conducting the mist through the liquid chamber and any liquid contained therein, the mist outlet conduit extending from the first end wall, through the liquid chamber, and through the fluid flow manifold to the sonication chamber, a first end of the mist outlet conduit being in fluid communication with a mist outlet port in the first end wall of the housing and a second end of the mist outlet conduit being in fluid communication with the sonication chamber, and the driver comprises: a substrate; an air flow sensor coupled to the substrate for detecting a change in air pressure relative to the ambient air pressure; at least one LED coupled to the substrate; and an air flow sensor holder coupled to the substrate, the air flow sensor holder including: a body portion having an opening for accepting the air flow sensor, the opening having a perimeter contacting the air flow sensor to form an airtight seal with the air flow sensor to create an ambient air space and an intake air space separated by the air flow sensor, wherein the body portion of the air flow sensor holder includes: a channel for supplying air at substantially ambient air pressure to the ambient air space; a further channel in fluid communication with an intake air flow path extending from the driver casing to the pod, the further channel for detecting an air pressure in the intake air flow path lower than the ambient air pressure, the lower air pressure being indicative of a user using the pod; the air flow sensor holder having a recess for accepting a respective one of the at least one LEDs, an intake air flow path extending from the air intake aperture in the driver casing to the sonication chamber, the intake air flow path passing through the bulkhead intake conduit in the driver, and the air intake conduit in the pod.

In some examples, the air flow sensor holder further comprises an auxiliary portion extending from the base portion, the auxiliary portion including a first side and a second side, the auxiliary portion covering each of the at least one LEDs and the second side of the auxiliary portion including the recess for accepting a respective one of the at least one LEDs, the auxiliary portion further including at least one projection extending from and substantially normal to the first side of the auxiliary portion.

In some examples, the projection is translucent such that, when the at least one LED is activated, light is visible through the projection.

In some examples, the substrate is a PCB.

In some examples, the driver includes a main PCB mounted therein, the substrate being the main PCB.

In some examples, the air flow sensor holder is formed of a single piece.

In some examples, the air flow sensor holder is of a resiliently deformable material.

In some examples, the air flow sensor holder is of Shore A 40 silicone.

In some examples, the at least one LED is a plurality of LEDs, and wherein a corresponding projection is provided for each LED.

According to an aspect of the present disclosure, there is provided an apparatus for controlling a driver for a mist inhalation pod for delivering a mist including nicotine, the apparatus comprising: a main printed circuit board (PCB) including: a plurality of conductive tracks providing an electrical connection between components mounted to the main PCB; a positive battery terminal to connect to a positive terminal of a battery; a ground battery terminal to connect to a ground terminal of the battery, the apparatus further comprising: a controller mounted to the main PCB and connected electrically to receive power via the positive battery terminal and the ground battery terminal, the controller including a processor and a memory, the memory storing executable instructions which, when executed by the processor, control at least one function of the apparatus; a load driver circuit mounted to the main PCB, the load driver circuit being configured to generate a load drive signal to control generation of the mist in the mist inhalation pod, the mist including nicotine; a first switch connected between the positive battery terminal and the load driver circuit, the first switch being controllable by the controller to: electrically connect the load driver circuit to the positive battery terminal when the load driver circuit is in use, and electrically disconnect the load driver circuit from the positive battery terminal when the load driver circuit is not in use; a fuel gauge circuit configured to monitor the charge level of the battery; and a second switch connected between the positive battery terminal and the fuel gauge circuit, the second switch being controllable by the controller to: electrically connect the fuel gauge circuit to the positive battery terminal when the fuel gauge circuit is in use, and electrically disconnect the fuel gauge circuit from the positive battery terminal when the fuel gauge circuit is not in use, wherein the controller remains connected electrically to receive power via the positive battery terminal and the ground battery terminal to control at least one function of the apparatus and the load driver circuit and the fuel gauge circuit are disconnected from the positive battery terminal when the load driver circuit and the fuel gauge circuit are not in use to minimise power consumption by the battery.

In some examples, the apparatus further comprises: a power supply input terminal configured to receive power from an external power supply; a power control circuit connected electrically to the power supply input terminal, the positive battery terminal and the ground battery terminal, the power control circuit being configured to control charging of the battery using power from the external power supply; a third switch connected between the positive battery terminal and the power control circuit, the third switch being controllable by the controller to switch on to connect the positive battery terminal to the power control circuit and to switch off to disconnect the positive battery terminal from the power control circuit; a fourth switch connected to the power supply input terminal, the fourth switch being configured to turn on the third switch when power is received at the power supply input terminal so that the power can charge the battery, the controller being configured to turn the fourth switch off in response to the controller receiving a signal indicative of an inhalation by a user on the mist inhaler so that power is drawn by the load driver circuit from the battery and not the external power supply during inhalation.

In some examples, the third switch has a body diode that conducts to provide a voltage to the power control circuit when the third switch is turned off to enable the power control circuit to monitor the charge level of the battery.

In some examples, the plurality of conductive tracks and the position of the components on the main PCB conduct currents across the main PCB in a plurality of current loops for delivering current at different current levels to circuits and sub-systems of the apparatus.

In some examples, the apparatus comprises: a conductive ground plane formed on the main PCB, the load driver circuit having a load driver circuit ground terminal which is connected electrically to the ground plane; and a shunt resistor which is connected electrically between the ground plane of the main PCB and the ground battery terminal.

In some examples, the ground plane extends across a majority of a side of the main PCB.

In some examples, the fuel gauge circuit is configured to detect a current flowing through the shunt resistor which is indicative of current flowing between the battery and the ground plane to enable the fuel gauge circuit to monitor the charge level of the battery based on the detected current flowing through the shunt resistor.

In some examples, the conductive tracks are spaced apart from one another, and the conductive tracks form an electrical connection with the conductive ground plane.

In some examples, the load driver circuit comprises at least one DC-DC converter circuit.

In some examples, the main PCB comprises an H bridge circuit having two AC outputs which are electrically connected to first ends of two respective AC conductive tracks of the plurality of conductive tracks of the main PCB, the two AC conductive tracks being positioned proximate to one another and terminating at two respective AC output terminals on the main PCB that are positioned proximate to one another so that electric fields generated by differential AC signals conducted by the AC conductive tracks at least partly cancel one another to minimise inductance in the AC conductive tracks.

According to an aspect of the present disclosure, there is provided a method for controlling a driver for a mist inhalation pod for delivering a mist including nicotine, the driver including a positive battery terminal to connect to a positive terminal of a battery, a ground battery terminal to connect to a ground terminal of the battery, a load driver circuit configured to generate a load drive signal, and a fuel gauge circuit configured to monitor the charge level of a battery, the method comprising: controlling a first switch connected between the positive battery terminal and the load driver circuit to: electrically connect the load driver circuit to the positive battery terminal when the load driver circuit is in use generating a load drive signal to control generation of the mist in the mist inhalation pod, the mist including nicotine, and electrically disconnect the load driver circuit from the positive battery terminal when the load driver circuit is not in use to minimise power consumption by the battery; and controlling a second switch connected between the positive battery terminal and the fuel gauge circuit to: electrically connect the fuel gauge circuit to the positive battery terminal when the fuel gauge circuit is in use, and electrically disconnect the fuel gauge circuit from the positive battery terminal when the fuel gauge circuit is not in use to minimise power consumption by the battery.

In some examples, the driver includes a power supply input terminal configured to receive power from an external power supply, and a power control circuit connected electrically to the power supply input terminal, the power control circuit being configured to control charging of the battery using power from the external power supply, the method further comprising: controlling a third switch connected between the positive battery terminal and the power control circuit to: switch on to connect the positive battery terminal to the power control circuit, and switch off to disconnect the positive battery terminal from the power control circuit; and controlling a fourth switch connected to the power supply input terminal to turn on the third switch when power is received at the power supply input terminal so that the power can charge the battery, and turn off the fourth switch in response to a signal indicative of an inhalation by a user so that power is drawn by the load driver circuit from the battery and not the external power supply during inhalation.

In some examples, the method comprises: detecting, using the fuel gauge circuit, a current flowing through a shunt resistor which is indicative of current flowing between the battery and a ground plane to enable the fuel gauge circuit to monitor the charge level of the battery based on the detected current flowing through the shunt resistor.

In some examples, the method comprises controlling the driver to: generate the load drive signal to control generation of the mist in the mist inhalation pod if a start condition is met; and not generate the load drive signal if the start condition is not met.

In some examples, the start condition is a start condition selected from a group including a battery charge level being below a threshold, a battery voltage level being below a threshold, an amount of the liquid in the pod being below a threshold and a temperature of the driver being above a threshold.

In some examples, the start condition is the status of a child lock for the driver, the child lock being controllable to prevent the driver being used by a user below a threshold age.

In some examples, the method comprises: communicating data between the driver and an application executing on a computing device; and using the application to control the operation of the driver.

In some examples, the method comprises: using the application executing on the computing device to verify at least one of an age and identity of a user by communicating with a remote computing device storing user data.

In some examples, the method comprises: using the application executing on the computing device to restrict use of the driver and monitor nicotine administration by the driver and the pod.

In some examples, the method comprises: using the application executing on the computing device to restrict use of the driver and the pod to a time frame.

According to an aspect of the present disclosure, there is provided an apparatus for controlling a driver for a mist inhalation pod for delivering a mist including nicotine, the apparatus comprising: a main printed circuit board (PCB) including: a plurality of conductive tracks providing an electrical connection between components mounted to the main PCB; a positive battery terminal to connect to a positive terminal of a battery; a ground battery terminal to connect to a ground terminal of the battery, the apparatus further comprising: a controller mounted to the main PCB and connected electrically to receive power via the positive battery terminal and the ground battery terminal, the controller including a processor and a memory, the memory storing executable instructions which, when executed by the processor, control at least one function of the apparatus; a load driver circuit mounted to the main PCB, the load driver circuit being configured to generate a load drive signal to control generation of the mist in the mist inhalation pod, the mist including nicotine; a switch connected between the positive battery terminal and the load driver circuit, the switch being controllable by the controller to: electrically connect the load driver circuit to the positive battery terminal when the load driver circuit is in use, and electrically disconnect the load driver circuit from the positive battery terminal when the load driver circuit is not in use; wherein the controller remains connected electrically to receive power via the positive battery terminal and the ground battery terminal to control at least one function of the apparatus and the load driver circuit is disconnected from the positive battery terminal when the load driver circuit is not in use to minimise power consumption by the battery.

According to an aspect of the present disclosure, there is provided an apparatus for controlling a driver for a mist inhalation pod for delivering a mist including nicotine, the apparatus comprising: a main printed circuit board (PCB) including: a plurality of conductive tracks providing an electrical connection between components mounted to the main PCB; a positive battery terminal to connect to a positive terminal of a battery; a ground battery terminal to connect to a ground terminal of the battery, the apparatus further comprising: a controller mounted to the main PCB and connected electrically to receive power via the positive battery terminal and the ground battery terminal, the controller including a processor and a memory, the memory storing executable instructions which, when executed by the processor, control at least one function of the apparatus; a fuel gauge circuit configured to monitor the charge level of the battery; and a switch connected between the positive battery terminal and the fuel gauge circuit, the switch being controllable by the controller to: electrically connect the fuel gauge circuit to the positive battery terminal when the fuel gauge circuit is in use, and electrically disconnect the fuel gauge circuit from the positive battery terminal when the fuel gauge circuit is not in use, wherein the controller remains connected electrically to receive power via the positive battery terminal and the ground battery terminal to control at least one function of the apparatus and the fuel gauge circuit is disconnected from the positive battery terminal when the fuel gauge circuit is not in use to minimise power consumption by the battery.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a diagrammatic view of a mist inhalation pod embodying the present disclosure;

FIG. 2 is a diagrammatic cross-sectional view of the mist inhalation pod, embodying the present disclosure;

FIG. 3 is a diagrammatic cross-sectional view of a part of the mist inhalation pod, embodying the present disclosure;

FIG. 4 is a diagrammatic exploded view of a part of the mist inhalation pod, embodying the present disclosure;

FIG. 5 is a diagrammatic view of a part of the mist inhalation pod, embodying the present disclosure;

FIG. 6 is a diagrammatic view of a part of the mist inhalation pod, embodying the present disclosure;

FIG. 7 is a diagrammatic view of the part of the mist inhalation pod illustrated in FIG. 6;

FIG. 8 is a diagrammatic view of a part of the mist inhalation pod, embodying the present disclosure;

FIG. 9 is a diagrammatic view of the mist inhalation pod, embodying the present disclosure;

FIG. 10 is a diagrammatic view of a part of the mist inhalation pod, embodying the present disclosure;

FIG. 11 is a diagrammatic cross-sectional view of the part of the mist inhalation pod illustrated in FIG. 10, embodying the present disclosure;

FIG. 12 is a diagrammatic cross-sectional view of a part of the mist inhalation pod, embodying the present disclosure;

FIG. 13 is a diagrammatic view of a part of a mist inhalation pod embodying the present disclosure;

FIG. 14 is a diagrammatic view of the part of the mist inhalation pod illustrated in FIG. 13, embodying the present disclosure;

FIG. 15 is a diagrammatic view of a inhalation pod and driver assembly, embodying the present disclosure;

FIG. 16 is a diagrammatic view of a driver, embodying the present disclosure;

FIG. 17 is a diagrammatic view of a driver, embodying the present disclosure;

FIG. 18 is a diagrammatic view of a subassembly of the driver, embodying the present disclosure;

FIG. 19 is a diagrammatic view a subassembly of the driver, embodying the present disclosure;

FIG. 20 is a diagrammatic view of a part of the driver, embodying the present disclosure;

FIG. 21 is a diagrammatic view of the part of the driver illustrated in FIG. 20, embodying the present disclosure;

FIG. 22 is a diagrammatic view of a part of the driver, embodying the present disclosure;

FIG. 23 is a diagrammatic view of the part of the driver illustrated in FIG. 22, embodying the present disclosure;

FIG. 24 is a diagrammatic view of a part of the driver, embodying the present disclosure;

FIG. 25 is a diagrammatic view of the part of the driver illustrated in FIG. 24, embodying the present disclosure;

FIG. 26 is a diagrammatic view of a part of the driver, embodying the present disclosure;

FIG. 27 is a diagrammatic view of the part of the driver illustrated in FIG. 26, embodying the present disclosure;

FIG. 28 is a diagrammatic view of a part of the driver, embodying the present disclosure;

FIG. 29 is a diagrammatic view of the part of the driver illustrated in FIG. 28, embodying the present disclosure;

FIG. 30 is a diagrammatic exploded view of the driver assembly, embodying the present disclosure;

FIG. 31 is a diagrammatic exploded view of the pod and the driver, embodying the present disclosure;

FIG. 32A is a diagrammatic cross-sectional view of the driver, embodying the present disclosure;

FIG. 32B is a diagrammatic cross-sectional view of the driver, embodying the present disclosure;

FIG. 33 is a diagrammatic view of a part of the driver, embodying the present disclosure;

FIG. 34 is a diagrammatic view of a part of the driver, embodying the present disclosure;

FIG. 35 is a diagrammatic view of a part of the driver, embodying the present disclosure;

FIG. 36 is a diagrammatic view of a part of the driver, embodying the present disclosure;

FIG. 37 is a diagrammatic view of a part of the driver, embodying the present disclosure;

FIG. 38 is a diagrammatic view of a part of the driver, embodying the present disclosure;

FIG. 39 is a diagrammatic cross-sectional view of the pod, embodying the present disclosure;

FIG. 40 is a diagrammatic cross-sectional view of a part of the pod, embodying the present disclosure;

FIG. 41 is a diagrammatic view of a part of the pod showing hidden lines, embodying the present disclosure;

FIG. 42 is a diagrammatic cross-sectional view of the pod and the driver, embodying the present disclosure;

FIG. 43 is a schematic diagram of components of a driver, embodying the present disclosure;

FIG. 44 is a circuit diagram of a DC-DC converter circuit of a driver, embodying the present disclosure;

FIG. 45 is a circuit diagram of a fuel gauge circuit of a driver, embodying the present disclosure;

FIG. 46 is a circuit diagram of high side switch circuits of a driver, embodying the present disclosure;

FIG. 47 is a diagrammatic view of a main printed circuit board (PCB) of a driver, embodying the present disclosure;

FIG. 48 is a schematic diagram of an integrated circuit of a driver, embodying the present disclosure;

FIG. 49 is a circuit diagram of a charging circuit of a driver, embodying the present disclosure;

FIG. 50 is a schematic diagram of a pulse width modulation generator of a driver, embodying the present disclosure;

FIG. 51 is timing diagram of an example of this disclosure;

FIG. 52 is timing diagram of an example of this disclosure;

FIG. 53 is a table showing port functions of an example of this disclosure;

FIG. 54 is a schematic diagram of an integrated circuit of a driver, embodying the present disclosure;

FIG. 55 is a circuit diagram of an H-bridge of an example of this disclosure;

FIG. 56 is a circuit diagram of a current sense arrangement of an example of this disclosure;

FIG. 57 is a circuit diagram of an H-bridge of an example of this disclosure;

FIG. 58 is a graph showing the voltages during the phases of operation of the H-bridge of FIG. 55;

FIG. 59 is a graph showing the voltages during the phases of operation of the H-bridge of FIG. 55;

FIG. 60 is a graph showing the voltage and current at a terminal of an ultrasonic transducer while the ultrasonic transducer is being driven by the H-bridge of FIG. 55;

FIG. 61 is a schematic diagram showing connections between integrated circuits of this disclosure;

FIG. 62 is a schematic diagram of an integrated circuit of this disclosure;

FIG. 63 is diagram illustrating the steps of an authentication method of examples of this disclosure;

FIG. 64 is a schematic diagram of an engine state machine of examples of this disclosure;

FIG. 65 is a schematic diagram of a pod authentication state machine of examples of this disclosure;

FIG. 66 is a schematic diagram of a pod authentication timeline of examples of this disclosure;

FIG. 67 is a schematic diagram of a pod and driver of examples of this disclosure;

FIG. 68 is a schematic diagram of an inhalation timeline of examples of this disclosure;

FIG. 69 is a schematic diagram of an inhalation timeline of examples of this disclosure;

FIG. 70 is a schematic diagram of an inhalation timeline of examples of this disclosure; and

FIG. 71 is a schematic diagram of a charging state machine of examples of this disclosure.

### DETAILED DESCRIPTION OF THE DISCLOSURE

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

The following disclosure provides many different embodiments, or examples, for implementing different features of the subject matter provided. Specific examples of components, concentrations, applications and arrangements are described below to simplify the present disclosure. These are, of course, merely examples and are not intended to be limiting. For example, the attachment of a first feature and a second feature in the description that follows may include embodiments in which the first feature and the second feature are attached in direct contact, and may also include embodiments in which additional features may be positioned between the first feature and the second feature, such that the first feature and the second feature may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The following disclosure describes representative arrangements or examples. Each arrangement or example may be considered to be an embodiment and any reference to an “arrangement” or an “example” may be changed to “embodiment” in the present disclosure.

Although the figures and description may indicate that there are two primary embodiments, each of the components of each embodiment are interchangeable where technically possible as it will be understood to be impractical to list or illustrate every possible permutation of components.

Conventional electronic vaporizing inhalers tend to rely on inducing high temperatures of a metal component configured to heat a liquid in the inhaler, thus vaporizing the liquid that can be breathed in. The liquid typically contains nicotine and flavorings blended into a solution of propylene glycol (PG) and vegetable glycerin (VG), which is vaporized via a heating component at high temperatures. Problems with conventional inhalers may include the possibility of burning metal and subsequent breathing in of the metal along with the burnt liquid. In addition, some may not prefer the burnt smell or taste caused by the heated liquid.

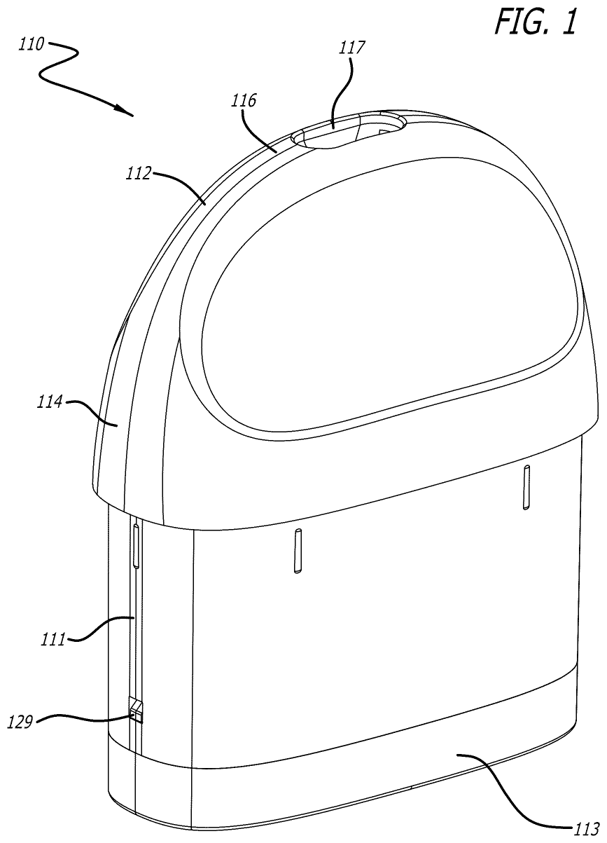

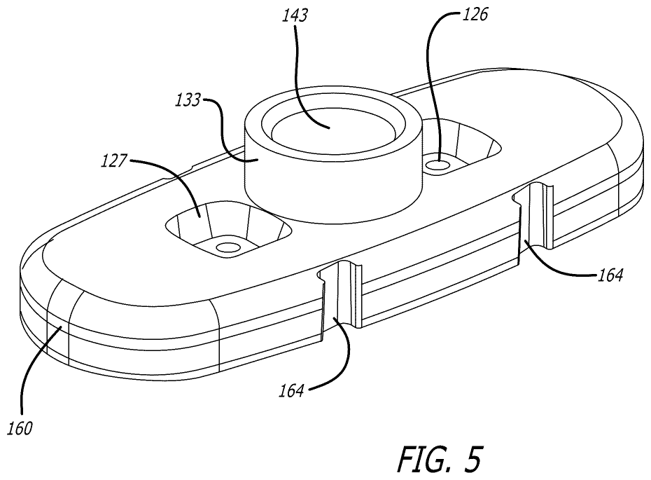

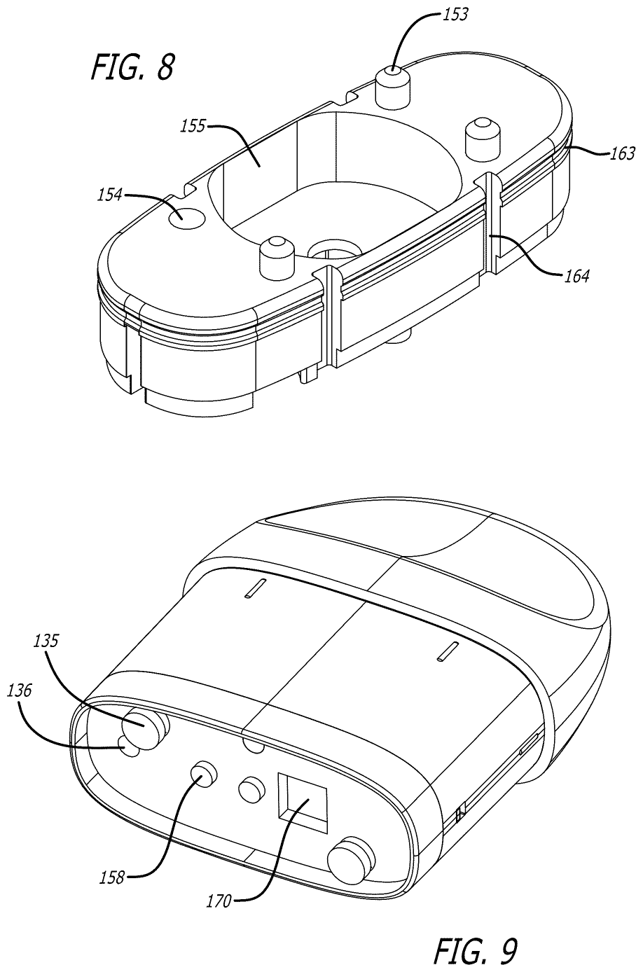

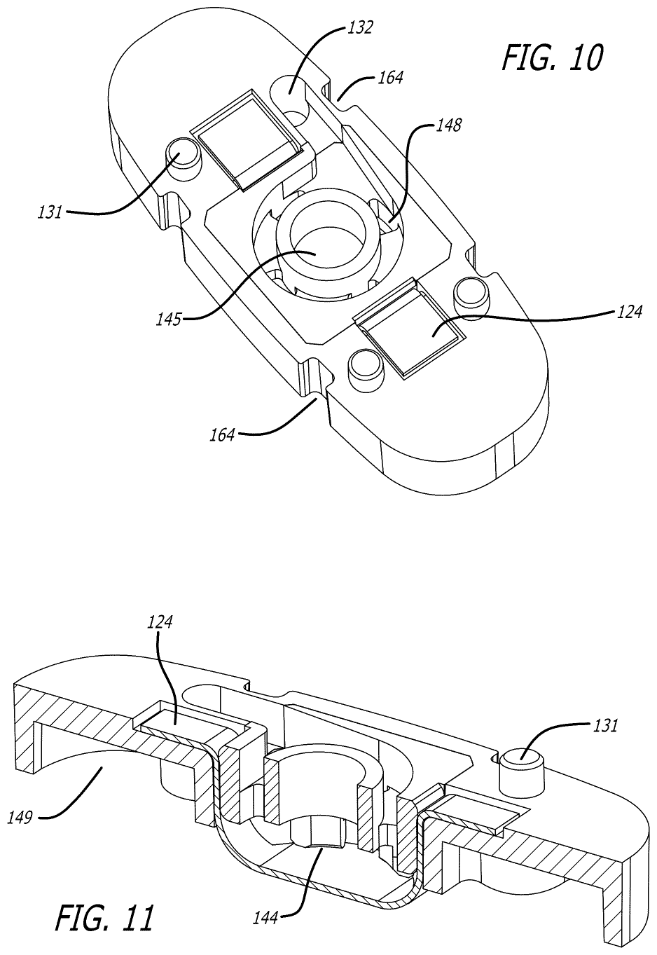

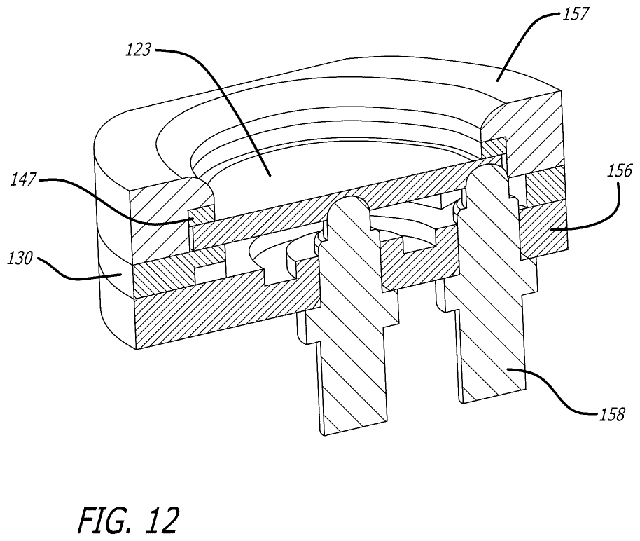

FIGS. 1 to 12 illustrate a pod 110, or components thereof, comprising a sonication chamber. It is noted that the expression “mist” used in the following disclosure means the liquid is not heated as in traditional inhalers known from the prior art. In fact, traditional inhalers use heating elements to heat the liquid above its boiling temperature to produce a vapor, which is different from a mist. A vapor involves a phase change from the liquid to a gas, whereas the liquid dispersed within the air in the present disclosure remains in the liquid phase.

When sonicating liquids at high intensities, the sound waves that propagate into the liquid media result in alternating high-pressure (compression) and low-pressure (rarefaction) cycles, at different rates depending on the frequency. During the low-pressure cycle, high-intensity ultrasonic waves create small vacuum bubbles or voids in the liquid. This phenomenon is termed cavitation. When the bubbles attain a volume at which they can no longer absorb energy, they collapse violently during a high-pressure cycle. During the implosion, very high pressures are reached locally. At cavitation, broken capillary waves are generated, and tiny droplets break the surface tension of the liquid and are quickly released into the air, taking mist form.

The following will explain more precisely the cavitation phenomenon.

When the liquid is atomized by ultrasonic vibrations, micro water bubbles are produced in the liquid.

The bubble production is a process of formation of cavities created by the negative pressure generated by intense ultrasonic waves generated by the means of ultrasonic vibrations.

High intensity ultrasonic sound waves leading to rapid growth of cavities with relatively low and negligible reduction in cavity size during the positive pressure cycle.

Ultrasound waves, like all sound waves, consist of cycles of compression and expansion. When in contact with a liquid, compression cycles exert a positive pressure on the liquid, pushing the molecules together. Expansion cycles exert a negative pressure, pulling the molecules away from one another.

Intense ultrasound waves create regions of positive pressure and negative pressure. A cavity can form and grow during the episodes of negative pressure. When the cavity attains a critical size, the cavity implodes.

The amount of negative pressure needed depends on the type and purity of the liquid. For truly pure liquids, tensile strengths are so great that available ultrasound generators cannot produce enough negative pressure to make cavities. In pure water, for instance, more than 1,000 atmospheres of negative pressure would be required, yet the most powerful ultrasound generators produce only about 50 atmospheres of negative pressure. The tensile strength of liquids is reduced by the gas trapped within the crevices of the liquid particles. The effect is analogous to the reduction in strength that occurs from cracks in solid materials. When a crevice filled with gas is exposed to a negative-pressure cycle from a sound wave, the reduced pressure makes the gas in the crevice expand until a small bubble is released into solution.

However, a bubble irradiated with ultrasound continually absorbs energy from alternating compression and expansion cycles of the sound wave. These cause the bubbles to grow and contract, striking a dynamic balance between the void inside the bubble and the liquid outside. In some cases, ultrasonic waves will sustain a bubble that simply oscillates in size. In other cases, the average size of the bubble will increase.

Cavity growth depends on the intensity of sound. High-intensity ultrasound can expand the cavity so rapidly during the negative-pressure cycle that the cavity never has a chance to shrink during the positive-pressure cycle. In this process, cavities can grow rapidly in the course of a single cycle of sound.

For low-intensity ultrasound the size of the cavity oscillates in phase with the expansion and compression cycles. The surface of a cavity produced by low-intensity ultrasound is slightly greater during expansion cycles than during compression cycles. Since the amount of gas that diffuses in or out of the cavity depends on the surface area, diffusion into the cavity during expansion cycles will be slightly greater than diffusion out during compression cycles. For each cycle of sound, then, the cavity expands a little more than it shrinks. Over many cycles the cavities will grow slowly.

It has been noticed that the growing cavity can eventually reach a critical size where it will most efficiently absorb energy from the ultrasound. The critical size depends on the frequency of the ultrasound wave. Once a cavity has experienced a very rapid growth caused by high intensity ultrasound, it can no longer absorb energy as efficiently from the sound waves. Without this energy input the cavity can no longer sustain itself. The liquid rushes in and the cavity implodes due to a non-linear response.

The energy released from the implosion causes the liquid to be fragmented into microscopic particles which are dispersed into the air as mist.

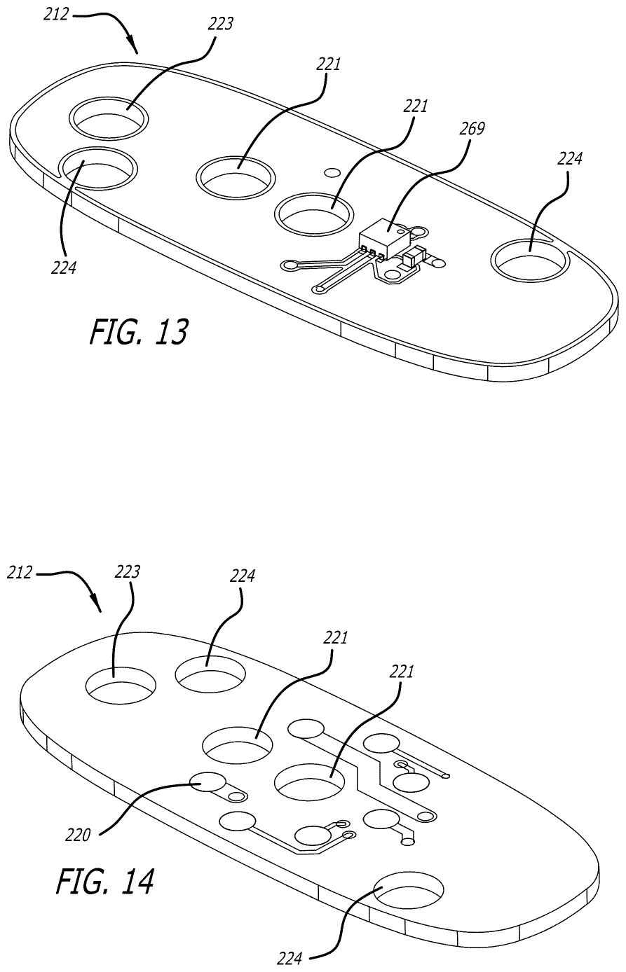

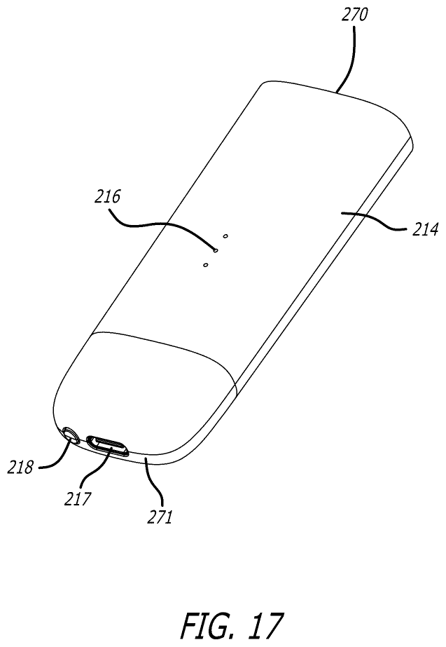

FIG. 1 shows a mist inhalation pod (hereinafter referred to as a pod) 110 according to some embodiments if the present disclosure. The pod 110 is configured to be releasably attached to a driver, one example of which is shown in FIGS. 13 to 36. The driver houses the components required to store electrical energy and provide an electrical signal to the pod 110. In other examples, the pod 110 may be fixed to, formed integrally with or otherwise non-releasably attached to the driver.

The pod 110 comprises a housing 111 having a first end and an opposite second end, a mouthpiece 112 and an end cap 113. Between the two ends of the housing 111 extends at least one side wall. In some examples, the housing 111 is of injection moulded plastic, specifically polypropylene that is typically used for medical applications. In some examples, the housing 111 is of a heterophasic copolymer. More particularly a BF970MO heterophasic copolymer is preferred, which has an optimum combination of very high stiffness and high impact strength. Parts moulded with this material also exhibit good anti-static performance.

A heterophasic copolymer such as polypropylene is particularly suitable for the pod housing 111 since this material minimises or does not cause condensation of the aerosol as it flows through the mouthpiece 112 to the user. This plastic material can also be directly recycled easily using industrial shredding and cleaning processes.

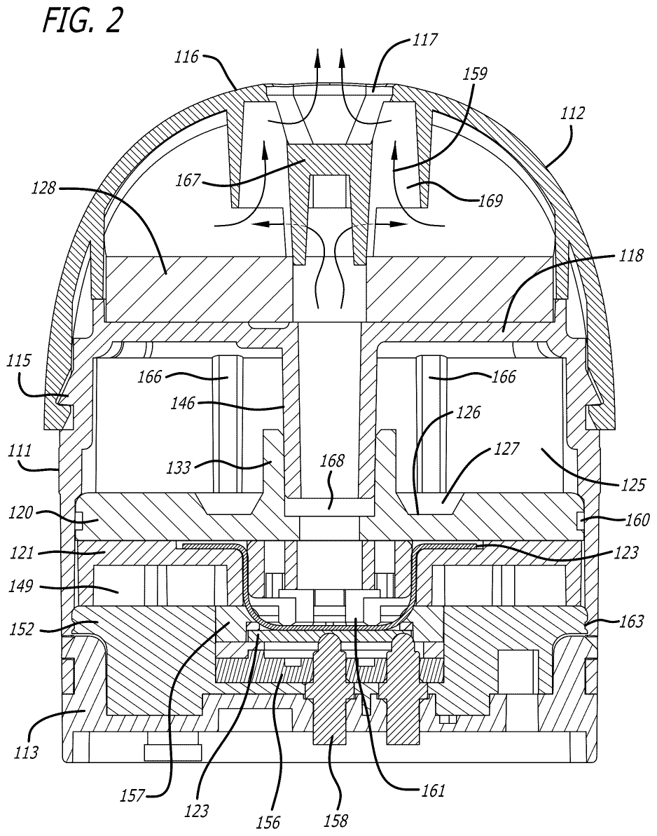

The mouthpiece 112 comprises a base 114 having an opening which receives a connector portion 115 positioned towards the first end of the housing 111 as seen in FIG. 2. The connector portion 115 may comprises at least one latch element that engages a latch recess to retain the mouthpiece 112 in connection with the housing 111.

The mouthpiece 112 may narrow progressively from the base 114 to a distal end 116. The distal end 116 comprises a mouthpiece outlet port 117 to enable mist to exit the pod 110 for inhalation by the user. The mouthpiece 112 may be substantially oval in cross section to allow for comfortable use by the user. The longer sides of the mouthpiece 112 may be indented in order to further increase the user comfort and experience.

The mouthpiece 112 may comprise an indirect flow path for mist produced by the pod 110 so that the mist takes a non-direct path to the mouthpiece outlet port 117. The mist is forced to take a path which is diverted radially outward and then radially inward again before passing through the mouthpiece outlet port 117. The mist flow path may further include a tangential component, or any flow path which increases the dwell time of the mist within the mouthpiece 112. One example of such a mist flow path is illustrated by the arrows 159 in FIG. 2. It will be understood that a non-direct flow path includes any flow path which includes a deviation from a straight line through the mouthpiece 112. A non-direct flow path reduces the likelihood of liquid droplets being drawn through the mouthpiece by the user, and increasing the dwell time allows for more liquid droplets to separate from the mist flow.

The mist preferably flows into the mouthpiece 112 through a mouthpiece inlet port which is in communication with a mist generating component. The mouthpiece 112 may act as a chamber through which the mist passes on its way to the mouthpiece outlet port 117. The mouthpiece chamber is of a larger diameter, and preferably of a much larger diameter, than the mist outlet conduit 146 and the mouthpiece outlet port 117.

A blocking element 167 may be positioned axially between the mouthpiece inlet port and the mouthpiece outlet port 117 to prevent the mist exiting the pod 110 straight through the mouthpiece outlet port 117 without first entering the mouthpiece chamber. The blocking element 167 may, as shown in FIG. 2, have a substantially horizontal plate portion which serves to interrupt the mist flow path. In some examples, the plate portion may not be horizontal and may instead be angled to deflect the mist flow in at least one specific direction. The blocking element 167 may further include at least one, but preferably a plurality of legs which extend longitudinally with respect to the pod 110. The legs may extend as far as an absorbing element 128 or the mist outlet conduit 146 to aid assembly and to prevent mist from bypassing the blocking element 167.

The blocking element 167 may further include at least one mist outlet aperture 169. The mist outlet aperture 169 is in fluid communication with both the mouthpiece chamber and the mouthpiece outlet port 117. The mouthpiece outlet aperture(s) 169 are preferably positioned at approximately 90 degrees to the mist flow path into the mouthpiece chamber, so as to increase the dwell time of the mist in the mouthpiece chamber. In other words, the legs of the blocking element 167 preferably block a direct path between the mouthpiece inlet port and the mouthpiece outlet aperture 169.

Although the blocking element 167 is described as a component, it could instead be integrated into the mouthpiece 112.

The end cap 113 attaches to the second end of the housing 111. The end cap 113 includes a plurality of deformable hooked elements 129 for receiving and securing to the side wall of the housing 111. The underside of the end cap 113 includes a recess, most clearly shown in FIG. 9, for housing circuitry such as an authentication PCB 212 as described herein and as illustrated in FIGS. 13 and 14.

FIG. 2 illustrates the internal components of the pod 110 according to some examples. The housing 11 comprises a first end wall 118 proximate the first end of the housing 111 and a second end wall in the form of the end cap 113. The first end wall 118 and the second end wall may be configured to close the first end and the second end of the housing 111, respectively. The side wall of the housing 111 may comprise at least one, but preferably a plurality of ridges 166, as shown in FIG. 2. The ridges 166 may be configured to engage corresponding longitudinal grooves 164 of the liquid barrier wall 120, the spacer 121, and the lower body portion 152. The ridge 166 and groove 164 interaction eases the assembly of the pod 110, where it is important that the internal components of the pod 110 align correctly with one another. If misalignment occurs, the air inlet conduit may not be correctly formed, thereby causing inefficiencies in the assembled pod 110. In some examples, at least one of the ridges 166 may be of differing size and/or shape to the others so that the pod 110 can only be assembled correctly without inadvertently placing a component incorrectly.

An absorbing element 128 may be positioned in or adjacent to the mist flow path so as to absorb any liquid droplets as the mist is conducted towards the mouthpiece 112. Preferably, the absorbing element 128 is at least partly of bamboo fibre. In some examples, the absorbing element 128 is positioned within the mouthpiece 112. The absorbing element 128 preferably extends radially to the wall of the mouthpiece 112 to increase the volume of mist droplets absorbed.

The pod 110 comprises a mist outlet conduit. In some examples, at least a first section 146 of the mist outlet conduit may be integrally formed with the housing 111 and extend from the first end wall 118 towards the second end wall.

The housing 111 may enclose a liquid barrier wall 120, a spacer 121, a fluid flow manifold 122, an ultrasonic transducer 123, and a capillary 124.

The liquid barrier wall 120 is positioned within the housing 111 and extends towards the side wall to create a seal between the liquid barrier wall 120 and the side wall of the housing 111. In some examples, the liquid barrier wall 120 has a contoured outer edge such that the outer edge contacts the side wall in two distinct locations, thereby providing a double seal against the side wall. The double seal may be provided by a circumferential groove 160. The liquid barrier wall 120 is spaced apart from the first end wall 118 to form a liquid chamber 125 therebetween. The liquid chamber 125 is configured to hold a liquid to be atomised. The liquid may comprise nicotine. In some examples, the liquid chamber has a volume of approximately 2.5 ml. In other examples, this may be reduced or increased, such as to comply with legal requirements or determined user preferences and requirements.

The liquid barrier wall 120 comprises a liquid channel 126 having a liquid inlet and a liquid outlet. The liquid channel 126 passes entirely through the liquid barrier wall 120 so as to allow liquid communication between the liquid chamber 125 and the capillary 124. The liquid barrier wall 120 may include more than one liquid channel 126 to improve the liquid flow rate to the capillary 124, or to improve the dispersion of the liquid over a larger surface area of the capillary 124. The diameter and the number of the liquid channels 126 are chosen to allow sufficient liquid flow to the capillary 124 without allowing a significant oversupply to the capillary 124, and as such prevent flooding and leaking, and also allow for more efficient mist generation.

The liquid barrier wall 120 may further comprise one or more recesses 127 in the planar face that defines the liquid chamber 125. Providing the liquid inlet of the liquid channel 126 in the recess 127 allows the liquid chamber 125 to be fully depleted of liquid before the pod 110 needs to be refilled or disposed of, due to the recess 127 representing the lowest point in the liquid chamber 125. As illustrated in FIG. 2, the recesses 127 and liquid channels may be positioned as close as possible to the sonication chamber 142 radially, thereby reducing the length of capillary 124 required and thus saving materials and manufacturing costs. Further, the recesses preferably span less than 50%, and more preferably less than 40% of the distance between the central protrusion 133 and the furthest point of the liquid barrier wall 120 from the central protrusion 133. Such dimensions may optimise the drainage of the liquid chamber 125.

The liquid barrier wall 120 may comprise blind holes in its lower surface (i.e. the planar surface opposite that on which the recess 127 is provided) configured to accept pegs 131 of the spacer 121.

The liquid barrier wall 120 may further comprise a central protrusion 133 which includes a through hole 143. The central protrusion 133 and through hole 143 form a second section of the mist outlet conduit.

The spacer 121 is positioned between the liquid barrier wall 120 and the second end wall within the housing 111. The spacer 121 has an outer wall forming a perimeter, and a hollow interior. Once the pod 110 is assembled, the perimeter of the spacer 121 extends towards the side wall of the housing 111. In order to save weight and material costs, the underside of the spacer, i.e., the side proximate the second end wall, may include at least one cavity 149.

The spacer 121 includes a slot 132 which may extend axially through the perimeter portion. In some examples, the slot 132 may not extend through the top surface of the perimeter portion, the top surface being the surface which may abut the liquid barrier wall 120. The slot 132 forms a section of the air inlet conduit once the pod 110 is assembled.

The spacer 121 further includes indentations 137 for accepting at least part of the second portion of the capillary 124. The indentations 137 may align with the liquid channels 126 of the liquid barrier wall 120 and thereby permit at least part of the second portion of the capillary 124 to lie adjacent the liquid outlet of the liquid channels 126. It will be appreciated that the spacer 121 may include only one indentation 137, or need not include indentations 137 at all.

In some envisaged alternative examples, the spacer 121 may be spaced from the lower surface of the liquid barrier wall 120. In a further alternative, indentations may additionally or alternatively be provided in the lower surface of the liquid barrier wall 120.

At least a part of at least one of the liquid barrier wall 120, the spacer 121, and the second end wall may be at least partly of a resiliently deformable material so as to prevent liquid leaking from the liquid chamber 125. Such a material may comprise silicone.

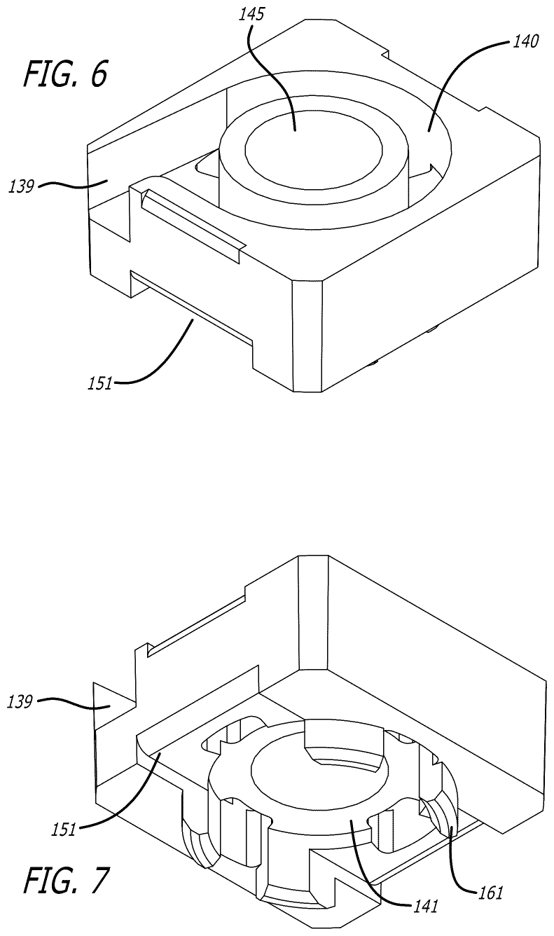

The hollow interior of the spacer 121 is sized to at least partially receive the fluid flow manifold 122. The manifold 122 has a first side and an opposite second side, the first side being the upper side as shown in FIG. 6, and the second side being the lower side shown in FIG. 7. When assembled, the first side of the manifold 122 is proximate the first end wall 118 and the second side of the manifold is proximate the second end wall.

The first side of the manifold 122 may comprise a channel 138. The channel 138 extends from an edge of the manifold 122 which is proximate the slot 132 in the spacer 121. The channel 138 may comprise a first portion 139 and a second portion 140. In some examples, the first portion 139 may be substantially straight and the second portion 140 may be at least partly annular. The first portion 139 may extend to the second portion 140 such that the air flow may transition from the first portion 139 to the second portion 140 smoothly, thereby minimising air turbulence within the manifold 122 that might otherwise affect the performance of the pod 110. The first portion 139 preferably extends to the second portion 140 tangentially or at an angle with respect to the second portion 140. The term “tangential” refers to an angle, such as an oblique angle, projecting from a part of the second portion 140. In the example in which the second portion 140 is at least partly annular, the first portion 139 extends tangentially or at a tangent relative to a curved part of the second portion 140.

Performance of the pod 110 may be affected by turbulent air flow due to the unpredictability of the direction, speed, and pressure of air within the pod 110. It is therefore preferable to avoid any features within the manifold 122 which may cause additional turbulence, such as sharp turns and edges. It will be appreciated that the manifold 122 shown in the figures is only one of a number of possible configurations. The first portion 139 may, for example, include a gentle curve. A gentle curve may allow for manufacturing and assembly practicalities, where certain other features of the pod 110 must be located in an optimal location for the first portion 139 of the channel 138.

The second side of the manifold 122 may include a cavity 141. Once the pod 110 is assembled, the cavity 141 partially defines a sonication chamber 142 in which the mist is produced.

The base of the cavity 141 includes a first aperture 145. The first aperture 145 extends through the centre of the annular channel 140 in the first side of the manifold 122. The first aperture 145 and the centre of the annular channel 140 thereby form a third section of the mist outlet conduit.

The base of the cavity 141 also includes one or more further apertures 148. The further apertures 148 extend from the base of the cavity 141 through to the second portion 140 of the channel 138 in the first side of the manifold 122. The further apertures 148 thereby allow air into the sonication chamber 142 at an angle transverse to the atomisation surface of the ultrasonic transducer 123. The air flow therefore contacts the ultrasonic transducer 123 with an increased force and may result in more efficient aerosolization and/or mist extraction. In some examples, the air flow is substantially perpendicular to the atomisation surface.

Preferably, there are four further apertures 148. In examples having two or more further apertures 148, the further apertures 148 are spaced, preferably evenly spaced, around the first aperture 145. The air inlet flow and the mist outlet flow may therefore be coaxial.

The one or more further apertures 148 of the air inlet conduit are preferably positioned radially inward of the edge of the atomisation surface. The apertures 148 may be of any shape, including circular holes or elongate slots. The apertures 148 are preferably of the same width and shape as the second portion 139 of the channel 138 so as to maximise air flow from the channel 138 to the sonication chamber 142.

In some examples, the second side of the manifold 122 may comprise protrusions 161, as shown in FIG. 7. In some examples, each protrusion 161 is a curved segment with the degree of curvature of the segment matching or substantially matching the degree of curvature of a part of an edge of the ultrasonic transducer 123. In some examples, there are four protrusions 161 in the form of quadrants that are positioned annularly and spaced apart from one another. The protrusions 161 may be positioned to contact the capillary 124 towards its outer edge. The protrusions 161 may therefore serve to keep the edge of the first portion of the capillary 124 in contact with the ultrasonic transducer 123, 223.

The protrusions 161 preferably extend around as much of the perimeter of the first portion of the capillary 124 as possible. Such a configuration reduces liquid leakage, and also controls the air flow through the manifold 122 to direct the air flow to the ultrasonic transducer 123. The gaps between the protrusions 161 therefore preferably only exist due to other features of the assembly, such as the further apertures 148 of the manifold 122 and the passage of the second portion(s) of the capillary 124. The protrusions 161 may further act to centre the manifold 122, and therefore the sonication chamber 142, over the ultrasonic transducer 123 during assembly.

Additionally or alternatively, a plurality of biasing elements (not shown) may extend from the base of the cavity 141 towards the second side of the manifold 122. The biasing elements are configured to urge the first portion of the capillary 124 into contact with the ultrasonic transducer 123 to enhance atomisation of the liquid. The biasing elements may be of any size, shape and material. Preferably, there are four biasing elements. Even more preferably, the biasing elements are evenly spaced across the surface of the capillary 124. The provision of the four biasing elements achieves a more even spread of a biasing force that acts against the first portion of the capillary 124 than other examples that comprise fewer than four biasing elements. The even spread of the biasing force of the four biasing elements ensures uniform contact between the first portion of the capillary 124 and the ultrasonic transducer 123. This optimises the transfer of ultrasonic waves generated by the ultrasonic transducer 123 to the liquid carried by the capillary 124, thereby helping to optimise the aerosolization of the liquid.

The capillary 124 extends between the liquid channel 126 of the liquid barrier wall 120 and the sonication chamber 142. In order to aid the passage of the capillary 124, the manifold 122 may include open slots 151 in its side surfaces. In some examples, the slots 151 may additionally or alternatively be positioned in the wall of the hollow interior of the spacer 121. Any change in angle of a slot, indentation, or otherwise which is configured to receive a portion of the capillary 124 may include a radius so as to not interfere with the fluid flow through the capillary 124.

The pod 110 further includes a lower body portion 152 positioned between the spacer 121 and the second end wall. The lower body portion 152 has an upper surface, a lower surface, and at least one side extending therebetween. The lower surface may abut the second end wall of the housing 111. The side(s) of the lower body portion 152 may, similar to the liquid barrier wall 120, have a contoured outer edge and a circumferential groove 163 such that the outer edge contacts the side wall in two distinct locations, thereby providing a double seal against the side wall. The upper surface may include dowels 153 configured to locate in corresponding holes (not shown) in the lower surface of the spacer 121. In some examples, the dowels 153 are integrally formed within the lower body portion 152. A through hole 154 may extend through both the upper and lower surfaces of the lower body portion 152. The through hole 154 serves as a section of the air inlet conduit.

The lower body portion 152 comprises a cavity 155 configured to accept the transducer holder 150. At least one, and preferably a plurality of passages, extend between the base of the cavity 155 and the lower surface of the lower body portion 152, the passages serving to allow electrical connections to pass therethrough. The lower body portion 152 may include recesses around its periphery so that when the pod is assembled, the hooked elements 129 are accommodated.

The transducer holder 150 is sized and shaped to be accepted by the lower body portion 152. The ultrasonic transducer 123 is supported in position adjacent to and in communication with the sonication chamber 142 by the transducer holder 150. The transducer holder 150 comprises a lower disc portion 156, a gasket 130, and an upper annular portion 157, at least one of which may comprise a resiliently deformable material.

The lower disc portion 156 may be generally planar. The lower disc portion 156 comprises holes through which electrical contacts 158 may extend to enable the transfer of a signal to the ultrasonic transducer 123. The lower disc portion 156 further comprises an annular ridge to act as the supporting surface for the underside of the ultrasonic transducer 123.

The upper annular portion 157 is sized and shaped to contact the gasket 130 and the spacer 121. The gasket 130 is preferably at least partially of silicone or another resiliently deformable material, and serves to seal the transducer holder 150 to minimise liquid leakage between the lower disc portion 156 and the upper annular portion 157. The upper annular portion 157 further acts to clamp the outer rim of the ultrasonic transducer 123 between itself and the annular ridge of the lower disc portion 156, via the gasket 130, such that any vibrations are efficiently transferred to the capillary 124, but preferably isolated from the housing 111. The upper annular portion 157 may incorporate a chamfer or radius on its inner edge, thereby aiding the change in direction of air flow within the sonication chamber 142 whilst reducing turbulence. During assembly, the chamfer or radius may also interact with the protrusions 161 of the manifold 122 to aid in centring the manifold 122 and sonication chamber 142 over the ultrasonic transducer 123.

Further, the face of the upper annular portion 157 which clamps the outer ring of the ultrasonic transducer 123 may include an annular retaining ring 147. The retaining ring 147 may be of silicone or another plastic material, and acts to minimise energy loss by the ultrasonic transducer 123 while still holding the ultrasonic transducer 123 securely in position.

The components of the transducer holder 150 may be attached to one another using heat staking (thermoplastic staking). In examples using heat staking to assemble the transducer holder 150, one of the lower disc portion 156 and the upper annular portion 157 may comprise posts (not shown), with the other of the lower disc portion 156 and the upper annular portion 157, along with the gasket 130, comprising holes for the posts to pass through. The silicone or other plastic material seals the components of the transducer holder 150 together to minimise the risk of liquid flowing between the components of the transducer holder 150, which may otherwise cause a malfunction.

The ultrasonic transducer 123 is configured to convert an electrical input signal into high frequency vibrations. The atomisation surface of the ultrasonic transducer 123 is adjacent to and in communication with the sonication chamber 142 via the capillary 124. In use, the atomisation surface is configured to turn the liquid, which saturates the capillary 124, into a mist.

The capillary 124 may be of any material capable of transporting liquid by capillary action. The shape of the capillary 124 may be determined by the channel formed between the manifold 122 and the spacer 121, the channel formed between the spacer 121 and the liquid barrier wall 120, and/or the shape of the ultrasonic transducer 123.

The capillary 124 comprises a first portion and a second portion. In some examples, the first portion is at least partly circular in shape to correspond with the shape of the ultrasonic transducer 123. By “partly circular” it is meant that the first portion preferably includes at least one arcuate edge extending between the second portions. In some examples, such as those depicted in the figures, two arcuate edges are provided, each arcuate edge extending from one of the second portions to another of the second portions.

In examples including more than one arcuate edge, they are preferably of the same length and/or radius, however this need not be the case. Uneven edges may be desired or necessary, for example, where a pod is non-symmetrical. It is preferable that the first portion covers substantially all of the atomisation surface so as to maximise the surface area available for liquid to be atomised.

The second portion includes at least one, but preferably a plurality of arms which extend away from the first portion. In examples employing a plurality of arms, the arms are preferable evenly spaced around the first portion. In preferred examples, the arms are generally rectangular in shape, although their shape may depend on the design and configuration of the pod 110 in which they are being implemented. For example, in the pod 110 illustrated in FIG. 2, the second portions extend upwards (i.e., transverse to the plane of the atomisation surface of the ultrasonic transducer 123), and then radially outward with respect to the first portion. The second portion may have any number of bends, as required by the design of the pod. In some embodiments the arms may be of differing lengths to one another. The first portion is at least partially superimposed on the atomisation surface of the ultrasonic transducer 123, and preferably substantially covers the atomisation surface of the ultrasonic transducer 123. The second portion is preferably adjacent the liquid outlet of the liquid channel 126, and more preferably covers at least a portion of the liquid outlet. More preferably still, the second portion completely covers all liquid channels 126 in the liquid barrier wall 120. The liquid from the liquid chamber 125 is therefore conducted from the liquid outlet of the liquid channel 126 to the atomisation surface of the ultrasonic transducer 123 by the capillary.

The first portion of the capillary may comprise an opening in the form of a hole or a slit. It will be understood that where the capillary of any embodiment is of a woven material, an opening is defined as being larger than the holes naturally found in the weave of the fabric.

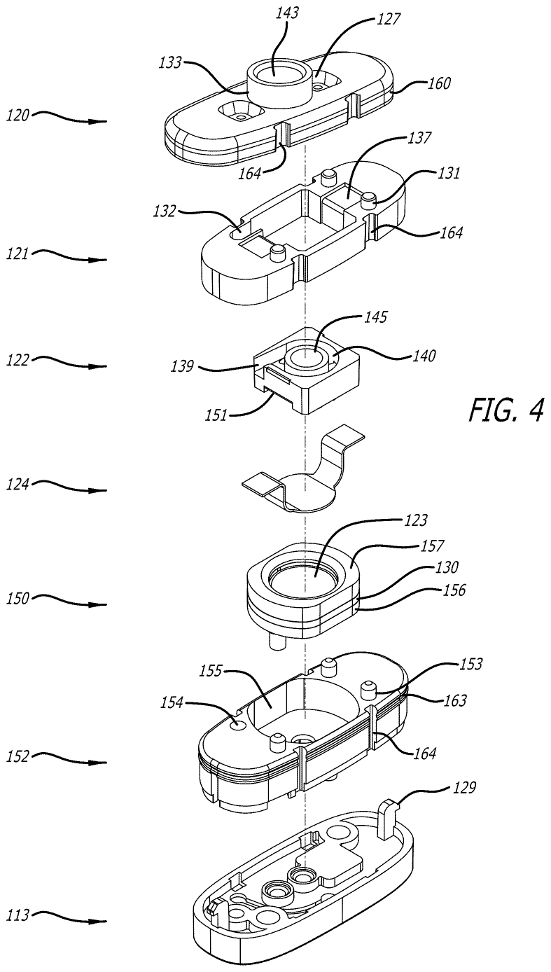

Referring now to FIG. 4, which illustrates an exploded view of part of the pod 110 and thus assists with the visualisation of the assembly. The transducer holder 150 is placed within the cavity 155 of the lower body portion 152, and the spacer 121 is coupled to the lower body portion 152. The dowels 153 of the lower body portion 152 engage in holes on the lower surface of the spacer 121. The dowels 153 and holes may be any of a clearance, transitional, or interference fit, and serve to prevent excessive movement of the lower body portion 152 relative to the spacer 121. The through hole 154 and the slot 132 align so as to form a part of the air inlet conduit.

The capillary 124 is inserted through the hollow interior of the spacer 121 so that the first portion of the capillary 124 is superimposed on the atomisation surface of the ultrasonic transducer 123. The second portions of the capillary 124 are positioned within the indentations 137 in the spacer 121.

The manifold 122 is positioned within the hollow cavity of the spacer 121, thereby forming the sonication chamber 142. The protrusions 161 and/or biasing elements urge the first portion of the capillary 124 into contact with the atomisation surface of the ultrasonic transducer 123. In examples utilising protrusions 161, said protrusions 161 may serve to centre the manifold 122 relative to the transducer holder 150. The second portion of the capillary 124 passes through a channel formed between the manifold slots 151 and the spacer 121. The first portion 139 of the manifold channel 138 aligns with the slot 132 in the spacer 121, further defining the air inlet conduit.

The liquid barrier wall 120 couples to the spacer 121 by means of pegs 131 on the spacer 121 engaging with holes in the underside of the liquid barrier wall 120. Similar to the dowels 153 in the lower body portion 152, the pegs 131 may engage with their respective holes by means of a clearance, transitional, or interference fit, and serve to prevent excessive movement of the liquid barrier wall 120 relative to the spacer 121.

The second portion of the capillary 124 is held in place within the indentations 137 in the spacer 121. Further, the slot 13 in the spacer 121, and both the first and second portions 139, 140 of the channel 138 in the manifold 122 are provided with a closing side by the liquid barrier wall 120, the air inlet conduit thereby defined.

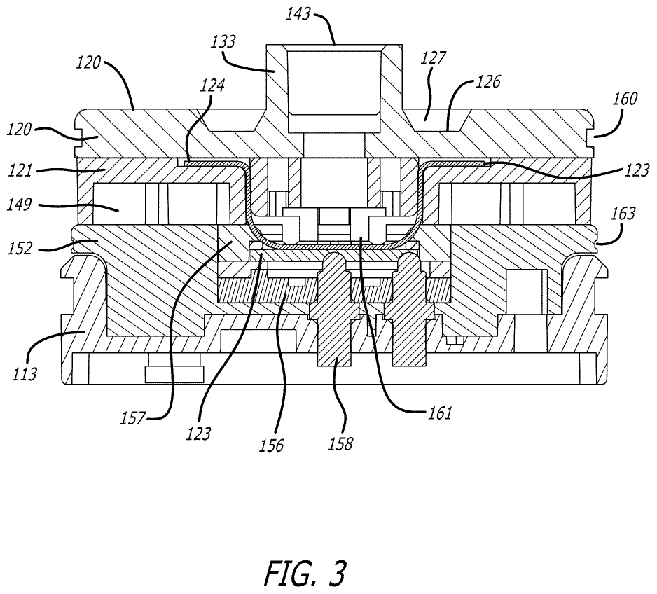

The end cap 113, the lower body portion 152, the transducer 123, and transducer holder 150, the spacer 121, the capillary 124, the manifold 122, and the liquid barrier wall 120 form the subassembly illustrated in FIG. 3. The various slots and indentations interacting with the capillary 124 may be shaped and/or sized in order to compress the capillary 124 a predetermined amount. The compressive force on the capillary 124 is low enough to avoid restricting liquid transfer, but high enough to prevent the liquid flooding the sonication chamber 142. The channel formed by the various slots and indentations are preferably spaced and sized to fit the capillary 124 without an air gap, so as to reduce leakage of the liquid and allow greater control of the flow of liquid being delivered to the sonication chamber 142. Alternatively, the capillary 124 may be shaped and sized to the design constraints of the components of the pod.

A collar 168 may be provided inside the central through hole 143 to encourage the seal between the liquid barrier wall 120 and the first portion of the mist outlet conduit 146. The collar 168 may also act as a reducer in examples where the outlet of the sonication chamber 142 and the first portion of the mist outlet conduit 146 are of a different diameter.

The abovementioned subassembly carries the advantage of being simple to manufacture, and also simple to assemble. For example, at least some of the various holes, channels, and protrusions are two dimensional forms, and not intricate and complex geometries.

The various sections are thus efficient to manufacture using well established manufacturing techniques, such as machining, casting, and moulding. This also means that manufacture and assembly may be at least partially autonomous. The parts of the device may be assembled using automated robots on a production line with minimal human intervention. The device is therefore configured to be mass produced on a production line relatively easily and at low cost compared with conventional mist generator devices.

The subassembly may be positioned within the housing 111 of the pod 110 such that the first section of the mist outlet conduit 146 is inserted into the central through hole 143 in the liquid barrier wall 120. The inner edge of the central protrusion 133 may be chamfered in order to aid insertion. The central protrusion 133 may have a stepped diameter and therefore act as a stop against the first portion 146 of the mist outlet conduit.

The end cap 113 may include a plurality of holes to provide continuity of the holes in the lower body portion 152. For example, the end cap 113 may include an air inlet hole 136, preferably configured to align with the through hole 154, which forms a portion of the air inlet conduit.

The absorbing element 128 and the mouthpiece 112 are coupled at the first axial end of the housing 111 to form the pod 110, an example configuration of which can be seen in FIG. 2.

The above-described pod assembly comprises both an air-tight air inlet conduit and an air-tight mist outlet conduit, each formed of multiple components of the pod 110. Air may be conducted to the sonication chamber 142 from proximate the second end wall via the through hole 154 in the lower body portion 152, the slot 132 formed in the spacer 121, the channel formed in the manifold 122, and through the one or more inlet apertures 148. After combining with the liquid particles, the mist exits the pod 110 through the first aperture 145 in the manifold 122, the first portion of the mist outlet conduit 146, the absorbent element 128, and the mouthpiece outlet port 117. The mist outlet conduit preferably passes through the liquid chamber 125.

Although the assembly has been described in a certain order, it will be appreciated that this is only an example and the components may be assembled in any plausible order. Similarly, terms such as “upper”, “lower”, and “side” are not to be construed as limiting, but for ease of reference to the figures.

The above-described pod 110 is configured to be coupled to a driver 210, the driver 210 comprising the means for powering and, in some embodiments, controlling the pod 110. The pod 110 is typically at least partially received by an axial end of the driver 210, and more specifically a cavity 211 in the driver 210. The pod 110, and more preferably the end cap 113, may include a seal around its lower edge to seal against the driver 210.

The end cap 113 may comprise retention pins 135 (seen in FIG. 9) for mounting a printed circuit board (PCB), hereinafter named an authentication PCB 212, to the pod 110. The authentication PCB 212 may be mounted to the pod 110 by any means, including but not limited to at least one of an interference fit with the retention pins 135 and/or the end cap 113, mechanical fixings or clips, and adhesive. In some examples, the authentication PCB 212 is mounted to the pod 110 to sit at least partly in the recess in the base of the pod 110. A microchip 269, 2690 may be carried by the authentication PCB 212 and be positioned so that the microchip 269, 2690 sits on a surface of the authentication PCB 212. The microchip 269, 2690 may therefore be positioned within a further recess 170 in the end cap 113 of the pod 110 when the authentication PCB 212 is mounted to the pod 110. Mounting the microchip 269, 2690 within the recess 170 is advantageous, as the recess 170 is able to protect the microchip 269, 2690 from impact when the pod 110 is mounted to a driver.

In some examples, the microchip 269, 2690 is a one-time-programmable integrated circuit (OTP IC) that may be used to identify and/verify the authenticity of the pod 110. Further details of the OTP IC 269, 2690 are provided herein.

The authentication PCB 212, shown in FIGS. 13 and 14, comprises an array of contacts 220 for receiving electrical signals from the driver 210. The authentication PCB 212 further includes a plurality of through holes, two of which may be retention pin receiving holes 224 configured to engage with the aforementioned retention pins 135. Two transducer driving holes 221 are provided and are preferably positioned amongst the array of contacts 220, thereby allowing an auxiliary PCB 222 having an array of pins to communicate with both the array of contacts 220 on the authentication PCB 212, and also communicate with the electrical contacts 158 which supply a drive signal to the ultrasonic transducer 123. The remaining through hole may be an air passage 223 configured to align with the air inlet hole 136 in the end cap, thereby allowing air into the pod to be combined with the atomised liquid particles in the sonication chamber 142.

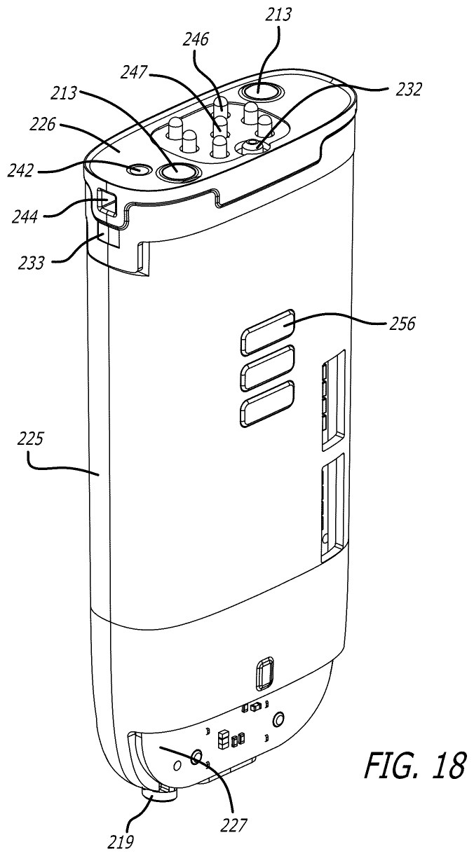

In some examples, the retention pins 135 are magnetic, and are configured to magnetically couple the driver 210 to the pod 110. In some examples, the retention pins 135 are magnets that are mounted to the pod 110 and arranged so that the ends of the retention pins 13 have opposite polarities to one another. In these examples, the polarities of the magnetic retention pins 135 must match the polarities of corresponding magnets 213, shown in FIG. 18 provided on the driver 210. The pod 210 may therefore only be coupled to the driver 210 in one orientation. The magnetic pins 135 repel the magnets 213 on the driver 210 when the pod 110 is moved towards the driver 210 in an incorrect orientation. This ensures that a user couples the pod 110 to the driver 210 in the correct orientation when replacing the pod 110. Correct orientation allows the electrical contacts and air flow path to line up correctly.

In examples where the pod 110 has an air inlet port in a position which is covered by the driver casing 214, the driver 210 may include a conduit to convey air from the surroundings to the air inlet port of the pod 110.

It will be appreciated that the pod 110 may be used with drivers different to those disclosed. Similarly, the driver 210 described herein may be used with various types of pods utilising ultrasonic mist generation, including those, for example, which incorporate a mesh within the nebuliser.

Mist inhalers are either disposable or reusable. The term “reusable” as used herein implies that a battery within the mist inhaler is rechargeable or replaceable or that the liquid within the pod is able to be replenished either through refilling or through replacement of a liquid tank. Preferably, the mist inhaler is reusable because both the battery is rechargeable and the liquid can be replenished, and is therefore more environmentally friendly than a single-use device.

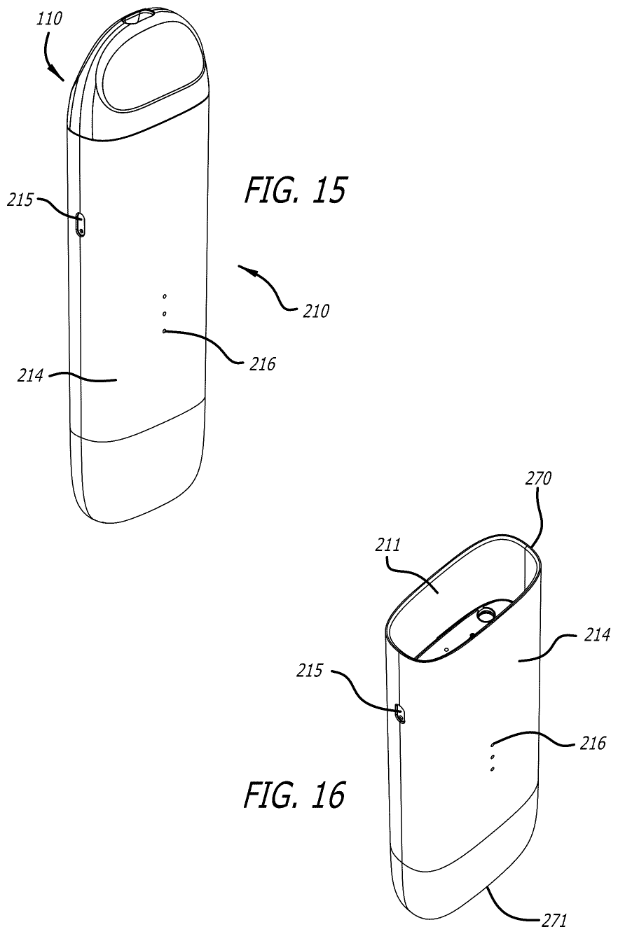

Referring now to FIGS. 15 to 17 of the accompanying drawings, the driver 210 comprises a driver casing 214. The driver casing 214 is preferably at least partly of metal. In some examples, the driver device housing 246 is entirely of aluminium (AL6063 T6) which protects the internal components from the environment (dust, water splashes, etc.) and also protects from damage from shocks (accidental drops, etc.). In some examples, the driver casing 214 may include vents that allow ambient air to enter the casing 214. The ambient air may provide a passive cooling effect to the electronics within the casing 214, thereby negating or reducing the active cooling requirements for the electronic components. Further passive cooling may be present in examples including a metal casing 214, as the casing 214 itself may act as a heat sink.

The driver casing 214 of this example comprises a generally tubular body having a substantially rectangular cross section. It will be appreciated, however, that any shape of casing is appropriate. The driver casing 214 is preferably a shell having an open end 270 and a base end 271, the open end 270 forming the cavity 211 in which the pod 110 is inserted. In some examples, the driver casing 214 may comprise a tapered section along which the cross sectional area of the tubular body reduces in size. In some examples, the tubular body may be at least partially of a certain material for aesthetic purposes, or to aid user comfort when gripping the driver 210.

The driver casing 214 includes a number of apertures which serve various functional purposes. An air intake aperture 215 is provided between the base end 271 and the open end 270. In some examples, the air intake aperture 215 is provided on the short side of the rectangular tubular casing 214, as shown in FIGS. 15 and 16. The air intake aperture 215 provides a passage for the surrounding air to enter the driver 210 and then the pod 110 when the user draws on the mouthpiece 112 of the pod 110. The air intake aperture 215 may be positioned on the short side of the casing 214 or otherwise, in order to not be blocked by the hand of a user holding the driver 210 naturally for use. The size of the air intake aperture 215 is configured to optimize air flow whilst not being too large to attract debris, and also takes into account the geometry of other pneumatic passages within the driver 210 and pod 110.

The casing 214 may further include a plurality of holes 216 positioned such that light originating from circuitry within the casing 214 can pass therethrough. The light may originate from LEDs of the device, and relate to any parameter of the device, such as charge level, power on/off, or liquid dose strength. LEDs are preferable due to their size and configurability.

The lower, distal end of the casing 214 may include a charger port opening 217 so that, in examples where the driver 210 is reuseable, the battery 228 within the driver 210 can be charged.

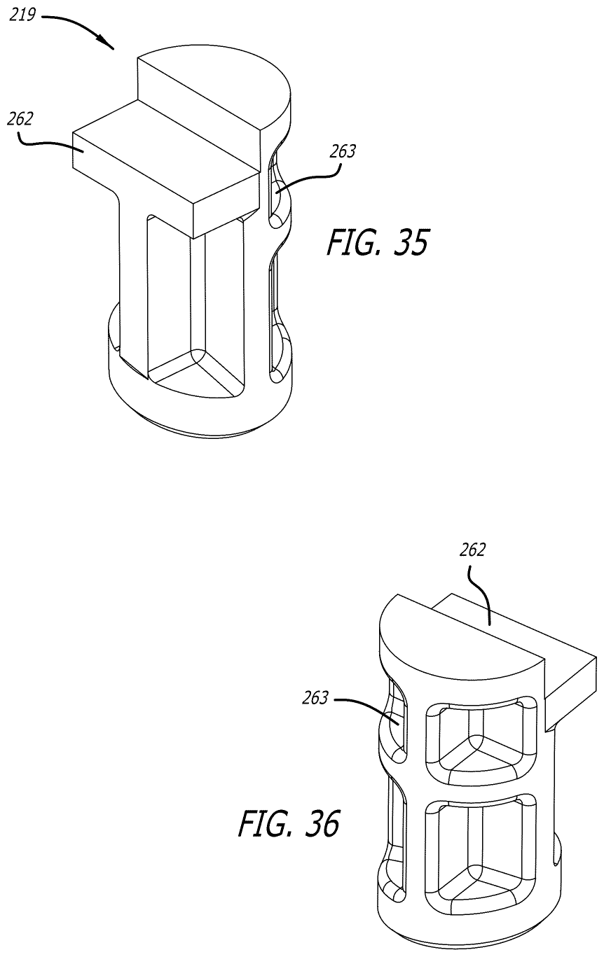

In some examples, a further opening 218 may be provided. This further opening 218 may be provided for housing a user-interactable button 219, the button 219 show in FIGS. 35 and 36, and described in further detail herein.

It will be noted that the positioning of any of the plurality of apertures in the casing 214 may be adjusted depending on the positioning of the functional components that lie beneath the casing 214.

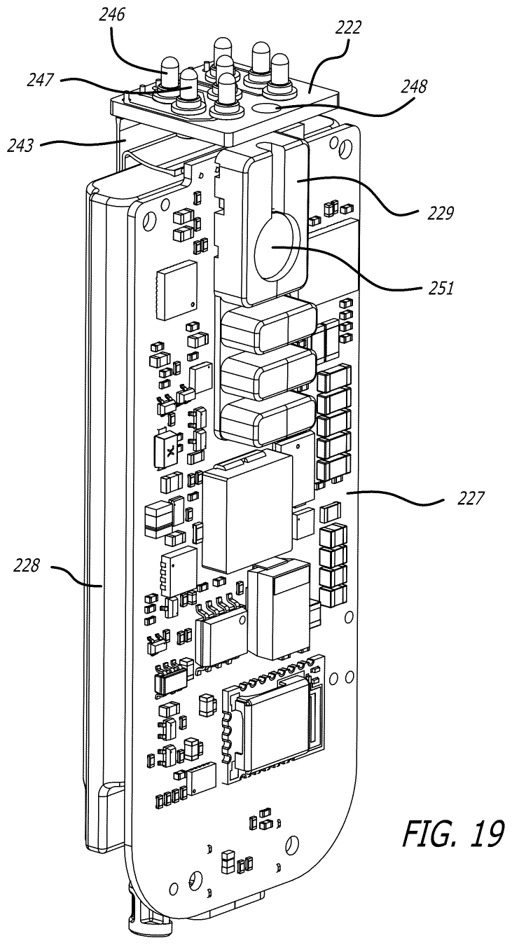

The subassembly shown in FIG. 18 comprises a skeleton 225, a bulkhead 226, and a main PCB 227. FIG. 19 shows the subassembly without the skeleton 225 and the bulkhead 226, and so further major components such as the auxiliary PCB 222, the main PCB 227, the battery 228, and the air flow sensor holder 229 can be seen. The components of the subassembly will now be described in turn.

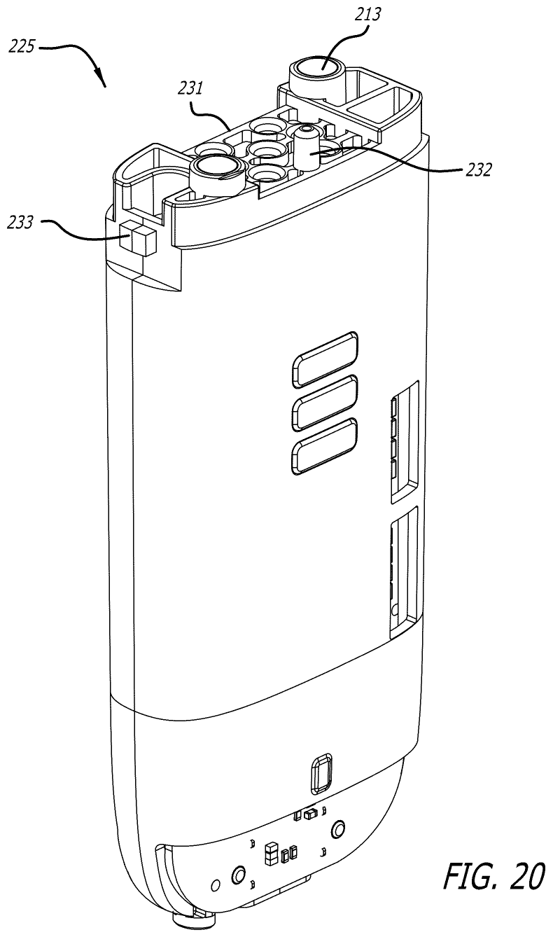

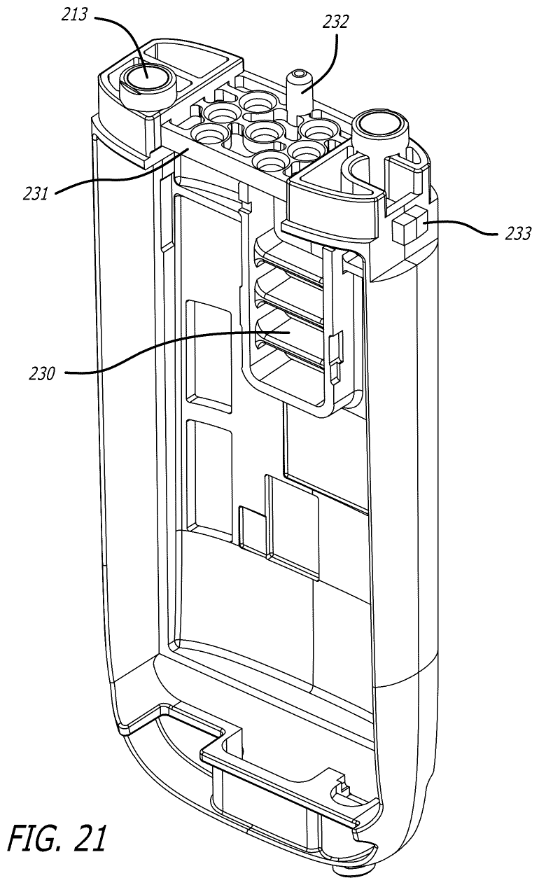

The skeleton 225 is shown in FIGS. 20 and 21, and serves as a frame for mounting the components of the driver 210 thereto. The skeleton 225 may then be inserted into and secured to the casing 214 upon assembly.

In some examples, the skeleton 225 is manufactured using industrial injection moulding processes. The moulded plastic skeleton ensures all parts are fixed and not loosely fitting inside the case. It also forms a cover over the front part of the main PCB 227. The skeleton preferably includes an open back (shown in FIG. 21) so as to decrease assembly time, optimise production efficiency and minimise material wastage.

A first end of the skeleton 225, i.e., the top of the skeleton 225 in FIGS. 20 and 21, includes a platform 231 on which the auxiliary PCB 222 is mounted. The platform 231 includes a number of recesses sized such that, once the auxiliary PCB 222 is mounted, the pins of the auxiliary PCB 222 may be supported axially, i.e., up and down relative to the skeleton 225. The recesses further improve the speed of assembly due to precise positioning of the auxiliary PCB 222 being possible.

An air flow sensor tube 232 may project from the platform 231 to allow the passage of air from the air flow sensor to be in fluid communication with the air intake flow path.

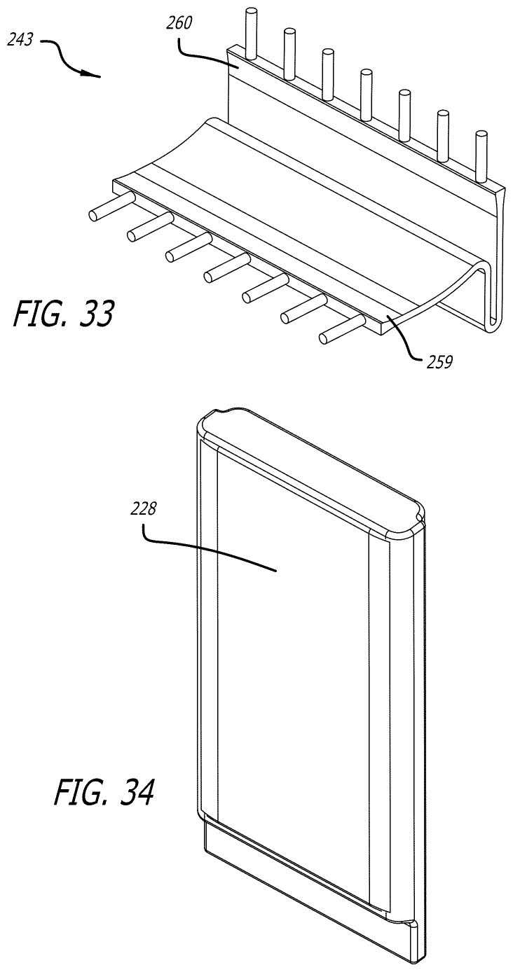

The platform 231 preferably does not extend the full depth of the skeleton 225 so as to allow an electrical cable to connect the main PCB 227 to the auxiliary PCB 222. Such a feature further improves assembly times, as the skeleton 225 may be slid into the casing 214 without fear of snagging on any components. One example of a suitable electrical cable is a flexible ribbon cable 243, as shown in FIG. 33. Of course, it will be appreciated that the platform 231 may extend the full depth of the skeleton 225 and instead be provided with a hole, slot, or other means of permitting electrical coupling of the main PCB 227 to the auxiliary PCB 222.

The front face of the skeleton 225, as viewed in FIG. 20, preferably has a similar profile to the casing 214 so as to maximise the available volume within the driver 210 for the components of said driver 210. The front face of the skeleton 225 may comprise a plurality of slots 230 configured to receive portions of the air flow sensor holder 229. The slots preferably align with LEDs on the main PCB 227, and also with holes 216 in the front face of the casing 214.

Other holes, slots, indentations and the like may be present to accommodate features and/or forms of the main PCB 227, the battery 228, or other components, or to improve air flow and cooling within the driver 210.

Each of the side faces of the skeleton 225 may include a protrusion 233, the protrusion 233 configured to engage in corresponding recesses or holes 234 in the bulkhead 226.

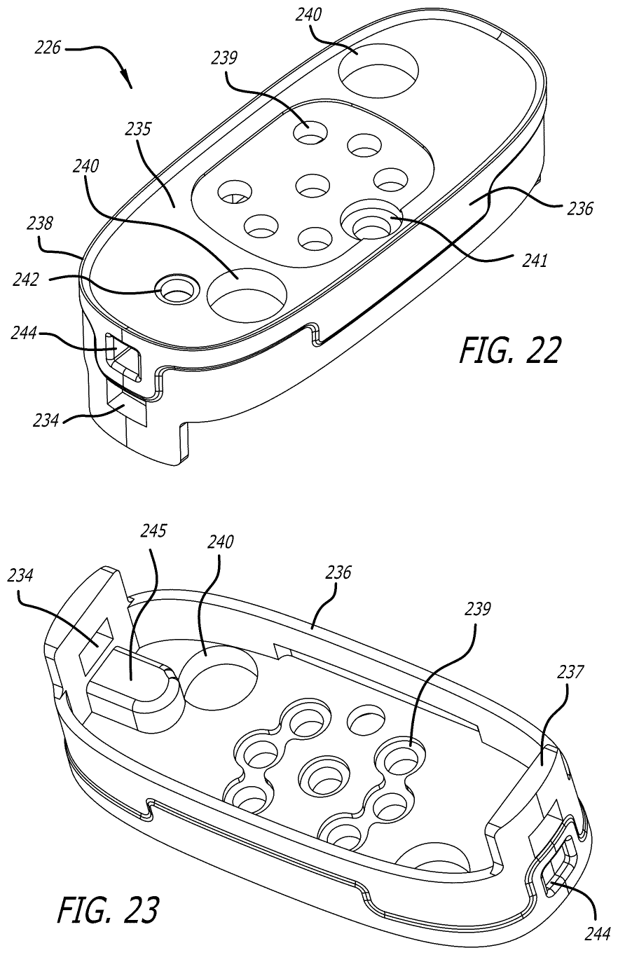

The bulkhead 226 is illustrated in FIGS. 22 and 23, and is shaped and sized to be couplable to the first end of the skeleton 225. The bulkhead 226 includes a body portion 235, a circumferential skirt 236, and legs 237 extending from the skirt 236.

The body portion 235 of the bulkhead 226 having a top surface and a bottom surface. A seal is preferably provided proximate, and more preferably at, the outer perimeter, i.e., the peripheral edge, of the top surface of the body portion. In some examples, the body portion 235 may have an upturned lip 238 around its upper peripheral edge. In examples including the upturned lip 238, a seal may be formed between the bulkhead 226 and the pod 110. In any of the above examples, the sealing element is sized and of a material to ensure a robust seal in order to minimise, and preferably prevent, air leaking between the sealed components.

The body portion 235 includes a plurality of apertures which pass through both the top and bottom surface. The apertures allow pneumatic, electrical, and magnetic communication between the driver 210 and the pod 110.

The electrical apertures 239 are shaped and sized to allow the pins of the auxiliary PCB 222 to pass therethrough once the bulkhead 226 is coupled to the skeleton 225. The pins of the auxiliary PCB 222 are therefore able to make contact with the authentication PCB 212 and the electrical contacts 158 which supply power to the ultrasonic transducer 123.

The magnetic apertures 240 allow for magnetic contact between the magnets 213 coupled to the driver 210 and the retention pins 135 coupled to the pod 110.

The two pneumatic apertures serve different purposes. There is provided an air flow sensor tube hole 241 to allow the air flow sensor tube 232 to pass through the bulkhead 226. There is also provided an outlet 242 of the bulkhead intake conduit 245, the bulkhead intake conduit 245 most clearly illustrated in FIG. 23.

The skirt 236 of the bulkhead 226 preferably extends generally transverse to the plane of the body portion 235, and serves both to secure the auxiliary PCB 222 to the skeleton 225, and to provide an inlet 244 for the bulkhead intake conduit 245.

At least the body portion 235 of the bulkhead 226 is preferably of a resiliently deformable material, and more preferably silicone. In some examples, the bulkhead is manufactured from a plurality of pieces, but in other examples the bulkhead 226 is formed or otherwise manufactured as a single piece.

It is preferable that the body portion 235 of the bulkhead 226 provides a hermetic seal against any components that pass therethrough such that as air passes through the driver 210, there are no leaks and so operation of the device is optimised. Such an arrangement may allow a chamber 268 to be formed between the body portion 235 of the bulkhead 226 and the authentication PCB 212 when the device is assembled. In the illustrated examples, the height of the chamber 268 is determined by the distance that the retention pins 135 protrude from the pod 110. It will be noted that in other examples, the magnets 213 may protrude from the driver 210, or a combination of the two. In some examples, the height of the chamber 268 may be determined by other elements of the pod 110 and/or driver 210, such as the upturned lip 238. In some examples, the underside of the mouthpiece 112 of the pod 110 seats on the leading edge of the open end 270 of the casing 214 to function as stop, thereby preventing further advancement into the casing recess 211. In some examples, the underside of the end cap 113 at least partially surrounds the chamber 268 and forms at least a part of the height of the chamber 268.

The size of the chamber 238 may be configured to at least one of: optimise the ability of the air flow sensor to detect a change is pressure; ensure a reliable seal around the periphery of the bulkhead 226; and/or improve the air flow to the sonication chamber 142 of the pod 110.

More specifically, it is preferable that the body portion 235 of the bulkhead 226 provides a seal around any component which passes through the bulkhead 226, such as: the magnets 213; the air flow sensor tube 232; the outlet of the bulkhead intake conduit 242; and the control signal connectors 246 and the drive signal connectors 247 of the auxiliary PCB 222. This prevents air from leaking out from around any component which passes through the bulkhead 226.

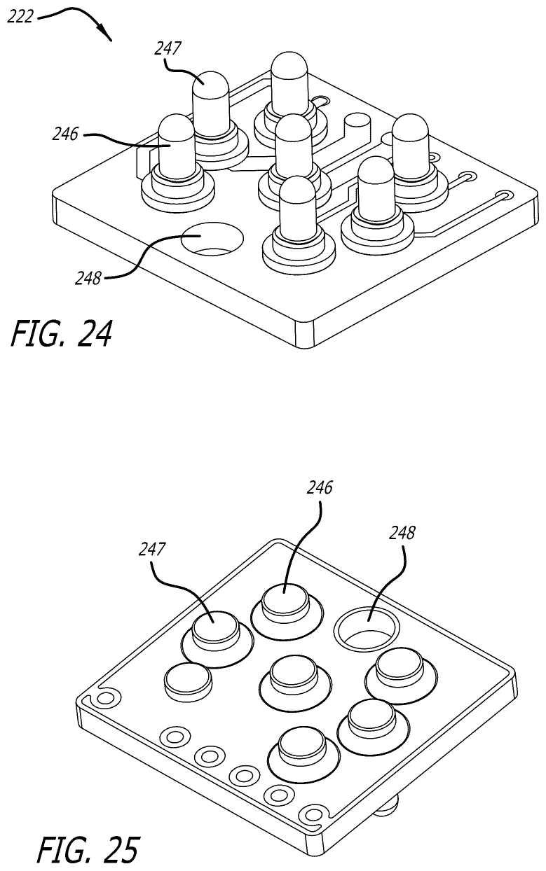

The auxiliary PCB 222 is illustrated in FIGS. 24 and 25. In this example, the auxiliary PCB 222 carries a plurality of control signal connectors 246 each configured to connect electrically to a respective contact 220 provided on the authentication PCB 212 to communicate control signals to the pod 110. In this example, the auxiliary PCB 222 also carries a plurality of drive signal connectors 247 each configured to connect electrically to a respective electrical contact 158provided in the pod 110 to communicate drive signals to the ultrasonic transducer 123. In this example, the drive signal connectors 247 extend through respective electrical through holes 239 in the bulkhead 226, and also through respective holes 221 in the authentication PCB 212. Direct contact between the drive signal connectors 247 and the electrical contacts 158 in the pod 110 allows for more efficient transmission of electrical signals and fewer possible points of failure, thereby improving reliability and longevity of the driver 210.

In this example, the control signal connectors 246 and the drive signal connectors 247 are spring-loaded pin connectors or pogo pin connectors. In examples of this disclosure, the control signal connectors 246 and the drive signal connectors 247 are each elongate with a contact surface at one end. In this example, the control signal connectors 246 may be pogo pins having a sealed base.

The drive signal connectors 247 are configured to deliver the drive signals at a current of up to 3 A, or more typically at up to 2 A. The drive signals are preferably AC drive signals at a frequency of 0.5 MHz to 1.5 MHz, 2.8 MHz to 3.2 MHz or 3 MHz to 5 MHz, depending on the application and required properties of the mist to be output from the pod 110.

In some examples, the authentication PCB 212 and the auxiliary PCB 222 provide passthrough electrical connections that provide an electrical connection between the control signal connectors 246 and the main PCB 227. This enables the electronic components, such as integrated circuits, on the main PCB 227 to receive signals from and send signals to the pod 110 via the control signal connectors 246.

The auxiliary PCB 222, similar to the bulkhead 226, may provide a through hole 248 through which the air flow sensor tube 232 may pass. It will be appreciated that instead of the hole 248, a slot, or cut-out could be provided. A further alternative is to provide an auxiliary PCB 222 having a shape such that the air flow sensor tube 232 does not pass through the auxiliary PCB 222. One edge of the auxiliary PCB 222 is configured to receive a second end 260 of the flexible ribbon cable 243 to convey electrical signals to and from the main PCB 227.

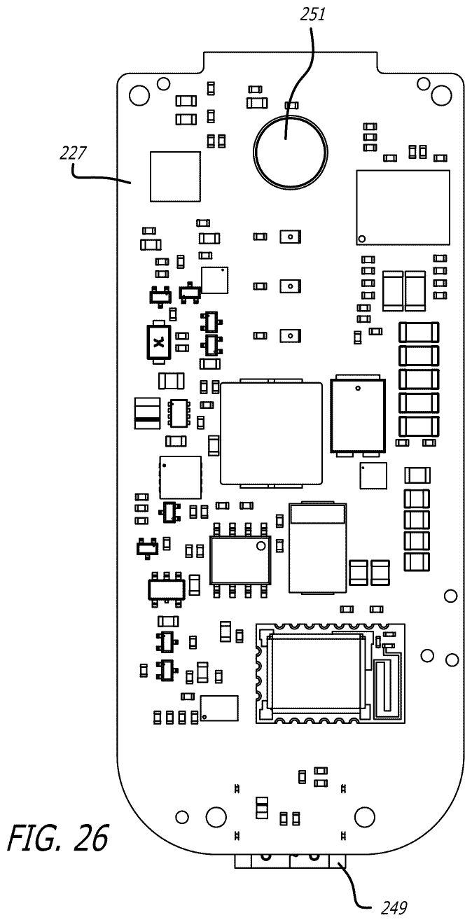

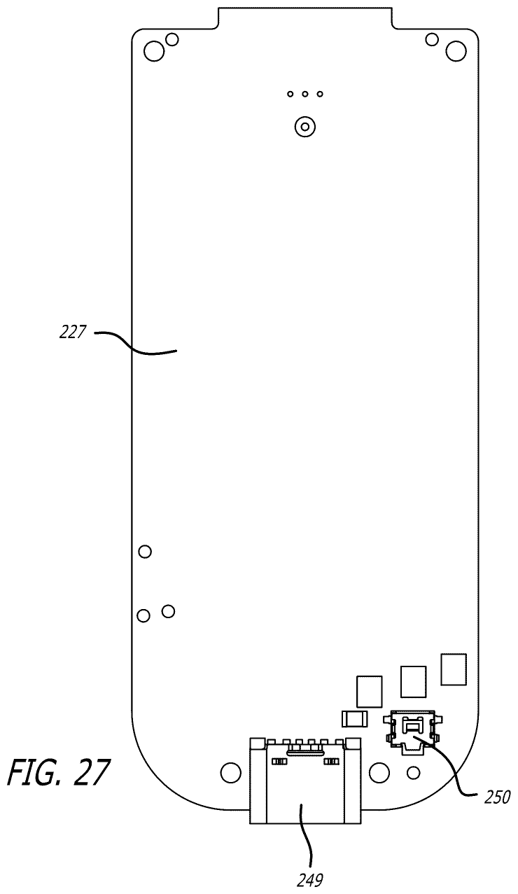

The main PCB 227 is shown in FIGS. 26 and 27. The main PCB 227 carries electronic components configured to generate control signals to control the operation of the pod 110 and drive signals to drive the pod 110 to generate a mist.

Some examples of electronic components that may be provided on the main PCB 227 are shown in FIGS. 26 and 27. However, it is to be appreciated that the main PCB 227 may incorporate other electronic components or may be configured differently from the examples described and illustrated herein. The main PCB 227 incorporates a processor, a memory, and other electronic components for implementing the electrical functions of the driver device 210.

The main PCB 227 may include various microchips and other components, such as: a H-bridge microchip for efficiently operating the ultrasonic transducer 123; an oscillator microchip; DC to DC converters; and microchip control units; low drop-out regulators; and capacitors for power smoothing and filtering. At least some of the above components are described further herein.

The rear of the main PCB 227, as shown in FIG. 27, may further include a charging port 249 (where a rechargeable battery 228 is implemented), and also an electronic switch button 250. The electronic switch button 250 preferably aligns with the further opening 218 in the casing 214 so that the button 219 may be attached to allow user interactions with the driver 210.

Each of the PCBs is preferably in the form of a laminated structure of conductive and insulating layers. Each conductive layer comprises a pattern of traces, planes and other conductive portions that provide electrical connections between components mounted to the PCB. Each PCB may be rigid or semi-rigid. Each PCB is preferably a multi-layer PCB but in other examples at least one of the PCBs may be a single-layer PCB.

In this example of the disclosure, the plurality of PCBs comprises the main PCB 227, the auxiliary PCB 222 and authentication PCB 212. In other examples of the disclosure, one or more of the PCBs may be omitted.

An air flow sensor 251 may be coupled to the main PCB 227. The air flow sensor 251 may be coupled directly to the main PCB 227 as shown in FIG. 26, or may instead be positioned elsewhere in the driver 210, such as on another substrate, and have wiring for communicating with the main PCB 227. The purpose of the air flow sensor 251 is to establish whether a user is drawing on the mouthpiece 112 of the pod 110 so that the ultrasonic transducer 123 may be powered to produce a mist. The air flow sensor 251 therefore activates the supply of power to the transducer 123 for sonication and aerosol production. The air flow sensor 251 detects a pressure drop to activate the driver device 210. In order for the air flow sensor 251 to detect a pressure drop, an air flow channel from the air flow sensor 251 may be in fluid communication with the air channel extending from the air intake aperture 215 of the casing 214 to the mouthpiece 112 of the pod 110.

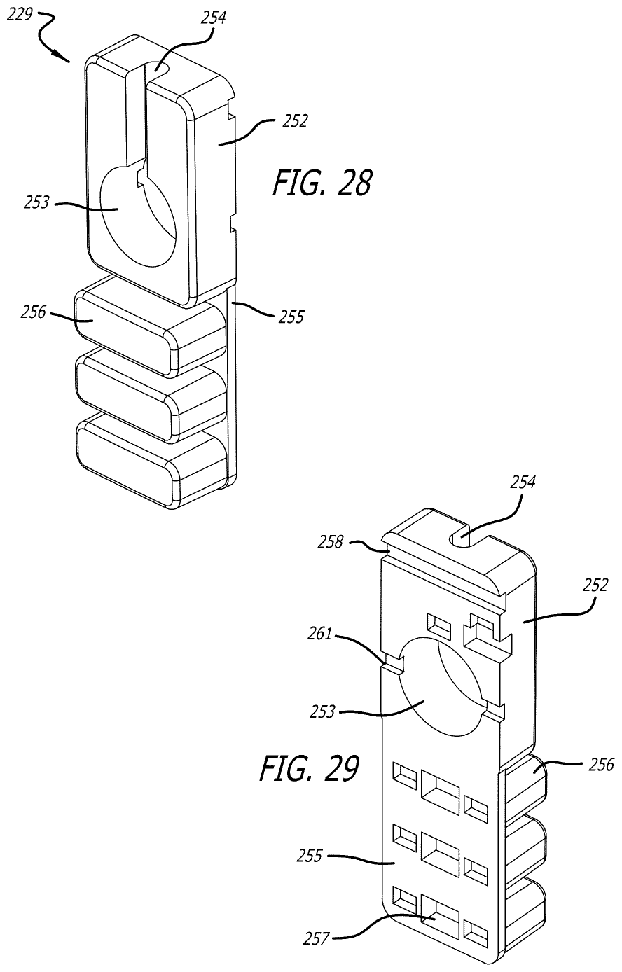

An air flow sensor holder 229, show in FIGS. 28 and 29, may be coupled to the main PCB 227 as shown in FIG. 19. The air flow sensor holder 229 is preferably manufactured or otherwise formed as a single piece to reduce manufacturing costs and improve assembly times. Preferably, the material of the air flow sensor holder 229 is a resiliently deformable material, such as silicone. More preferably, the material is Shore A 40 silicone.

The air flow sensor holder 229 may include a main body 252. The main body 252 may be generally a cuboid or rectangular prism in shape. An opening 253, which may be a through hole or recess, may be provided which is sized and shaped to compliment the size and shape of the air flow sensor 251 such that once the driver 210 is assembled, the air flow sensor 251 lies and is sealed within the opening 253. A channel 254 may extend radially from the opening 253 to the edge of the main body 252 and define an intake air space. It will be appreciated that although the channel 254 is depicted as an open slot that forms a conduit upon assembly with the skeleton 225, the channel 254 may, in some examples, instead form a self-contained conduit owing to a closed slot or a hole of circular cross section. Further, and depending on the layout of the driver 210, the channel 254 could incorporate curves and bends as necessary. In some examples, the air flow sensor tube 232 may be integrally formed with the air flow sensor holder 229.

The channel 254 is preferably dimensioned such that it terminates at one end of the air flow sensor tube 232 of the skeleton 225 upon assembly. There is thereby provided a part of the air sensor flow path from the air flow sensor 251 to tap into the air channel and detect the suction provided by the user.

The air flow sensor holder 229 preferably incorporates an auxiliary portion 255 which extends from the base of the main body 252. The substrate has a first side and a second side. At least one, but preferably a plurality of projections 256 are provided, which preferably extend from and substantially normal to the first side of the auxiliary portion 255. The projections 256 are configured to accept LEDs on the main PCB 227, and align with the LED holes 216 in the driver casing 214 once the driver 210 is assembled. Preferably, the projections 256 are distinct projections to avoid cross bleeding of light therebetween. It is preferred to avoid the cross bleeding of light, as this would be visible to the user and could cause confusion.

Turning now to FIG. 29, which shows the rear of the air flow sensor holder 229. The above-mentioned LEDs are accepted by the projections 256 through recesses 257 in the second side of the auxiliary portion 255. The depth of the recesses 257 and the height of the protrusion may be chosen such that some light originating from the LEDs is visible through the air flow sensor holder 229, i.e., at least the projections 256 are translucent.

The rear of the air flow sensor holder 229 may also include a track 258 which extends across its entire width. The track 258 in this example is to allow space for a first end 259 of the flexible ribbon cable 243 to connect to the main PCB 227.

In some examples, the rear of the air flow sensor holder 229 includes one or more channels 261 which extend from the opening 253 to the edge of the main body 252 of the air flow sensor holder 229 to define an ambient air space. These channels 261 allow air which is under substantially atmospheric or ambient pressure to reach the air flow sensor 251, which means that the air flow sensor 251 has a reference value to measure the air pressure in the intake air flow path against. The driver 210 is therefore able to operate correctly in different locations and at different altitudes, for example. Ambient pressure will be understood to mean the air pressure immediately surrounding the driver 210.

The rear of the air flow sensor holder 229 may further include any number of recesses or holes to accommodate components of the main PCB 227 or the geometry of the skeleton 225.

The driver 210 further comprises a battery 228 and a battery power connector (not shown) which is attached to the battery 228. In this example, the battery 228 is superimposed on the main PCB 227. The width of the battery 228 is preferably less than or equal to the width of the main PCB 227 and the length of the battery 228 is preferably less than or equal to the length of the main PCB 227. Such a battery size is considered appropriately large enough to last a predetermined amount of time without making the driver 210 too large or heavy. The battery power connector is secured to the main PCB 227, preferably by a least one cap connector.

In some examples, the battery 228 is a 3.7V DC Li—Po battery with 1140 mAh capacity and 10C discharge rate. A high discharge rate is required for voltage amplification of up to 15V that is required by the ultrasonic transducer 123 for desirable operation. The shape and size of the battery is designed, within physical constraints, as per the shape and size of the device and space allocated for the power source.

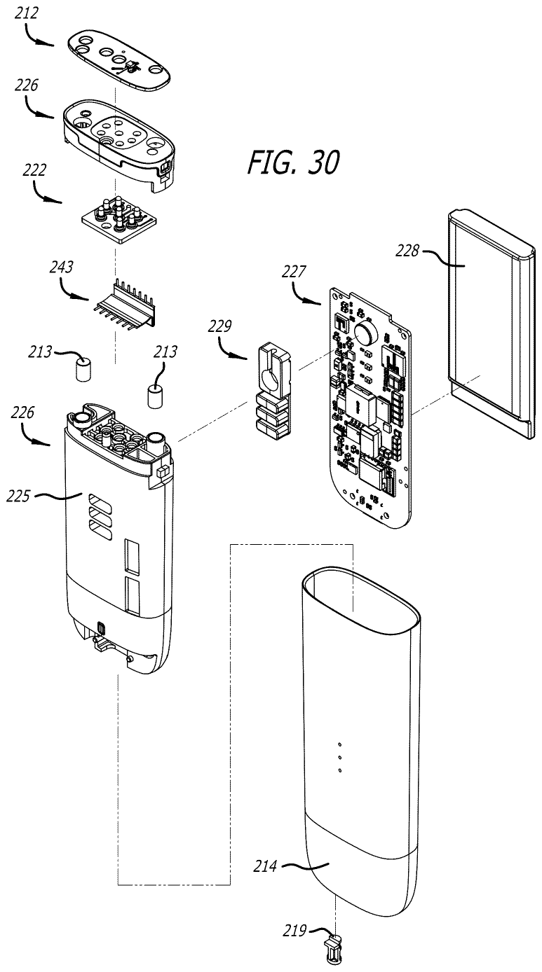

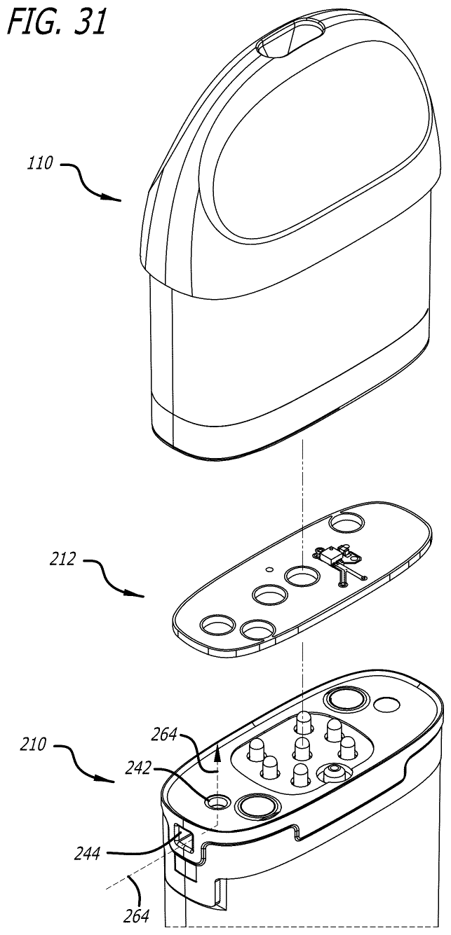

Turning now to the assembly of the driver 210, and to FIG. 30 which shows an exploded view of the driver 210. It will be appreciated that the following assembly description need not be carried out in the exact order described, and that other orders of assembling components are possible.

The air flow sensor holder 229 is coupled to the main PCB 227 such that the rear side of the air flow sensor holder abuts the main PCB 227. The intimate coupling of these two components minimises LED light cross bleed between the projections 256, and also seals the air flow sensor 251 against the main PCB 227. In this way, the only route that air can take to reach the lower portion of the air flow sensor 251 is through the channels 261.

The battery 228 is electrically coupled to the opposite side of the main PCB 227 than the air flow sensor 251, and the battery 228 is preferably positioned in a plane parallel to that of the main PCB 227. This leads to a space-efficient and compact assembly.

The first end 259 of the ribbon cable 243 may then be connected to the main PCB 227. The subassembly including the main PCB 227, the air flow sensor holder 229, the battery 228, and the ribbon cable 243 can then be inserted into the rear side of the skeleton 225 such that the projections 256 of the air flow sensor holder 229 are received within the slots 230 in the skeleton. In some examples, the subassembly may be clipped or otherwise fixed in position. In some examples, an adhesive may be used instead of or in addition to any other fixing.

The auxiliary PCB 222 may then be lowered onto the platform 231, with the air flow sensor tube 232 protruding through the hole 248 in the auxiliary PCB 222. The auxiliary PCB 222 therefore preferably lies in a plane transverse to the main PCB 227 and the device as a whole. This is so that the contact surfaces of the control signal connectors 246 and the drive signal connectors 247 terminate in a contact plane to align with the authentication PCB contacts 220 and the contacts 158 on the pod 110 which power the transducer 123.

The contact plane is transverse, perpendicular or generally perpendicular to a plane of the main PCB 227 and the contact plane faces outwardly into the cavity 211 of the casing 214. Consequently, when the pod 110 is inserted into the cavity 211 in the driver casing, the control signal contacts and the drive signal contacts are moved in a plane parallel to or generally parallel to the contact plane. The control signal contacts and the drive signal contacts then come into contact with the control signal connectors 246 and the drive signal connectors 247 at substantially the same time as one another. Moving the control signal contacts and the drive signal contacts directly towards and onto the contact surfaces of the control signal connectors 246 and the drive signal connectors 247 avoids the need to slide the contact surfaces against the control signal contacts and the drive signal contacts. This minimises mechanical wear to the contact surfaces and the control signal contacts and the drive signal contacts. Consequently, the longevity of the driver 210 and the pod 110 are improved since the contact surfaces and the control signal contacts and the drive signal contacts can remain intact for longer with minimal mechanical wear.

After the auxiliary PCB 222 has been mounted to the platform 231, the second end 260 of the ribbon cable 243 can be electrically coupled to the auxiliary PCB 222, thereby establishing the electrical connection between the main PCB 227 and the auxiliary PCB 222.

Next, magnets 213 are placed in recesses in the skeleton 225 on either side of the platform 231. The magnets 213 may be retained by adhesive or, additionally or alternatively, by mechanical means, such as the bulkhead 266.

The bulkhead 226 is coupled to the skeleton via the complementing protrusions 233 on the skeleton 225 and recesses 234 in the bulkhead 226. It should be noted that other fixing means may be used in some examples. In examples where the bulkhead 226 is formed of more than one piece, the bulkhead may be assembled before coupling to the skeleton 225.

Once the bulkhead 226 is coupled to the skeleton, the components passing through any holes in the bulkhead 226 may be hermetically sealed as described above.

The above-described example assembly procedure forms the subassembly shown in FIG. 18. The casing 214 may then receive the skeleton 225 and the components coupled thereto, thereby forming the driver 210 having an airtight seal between the air intake aperture 215 and the bulkhead intake conduit 245. The bulkhead intake conduit 245 is thereby formed entirely within the driver 210.

The base end 270 of the casing 214 may then receive the button 219. An example of a button 219 is shown in FIGS. 35 and 36. The button 219 preferably has a shape and size complementary to the shape and size of the opening 218 in the casing 214. The button may include protrusions 262 configured to make contact with the electronic switch button 250, and thus allow the user to interact with the driver 210. A push or hold of the button may, for example, allow the user to check the remaining battery life, check the remaining liquid volume, and/or initiate a Bluetooth pairing process. The recesses 263 in the button 219 are sized to be slightly longer than complimentary ribs in the driver casing 214. The recesses 263 being oversized allows for axial movement of the button 219. The amount of axial movement is configured to allow a positive user experience, and also to reduce false press instances when the driver is in a pocket or a bag, for example. In some examples, at least one of the button 219 and/or opening 218 may be sized and/or shaped such that once the button 219 is inserted into the opening 218, it cannot be easily removed.

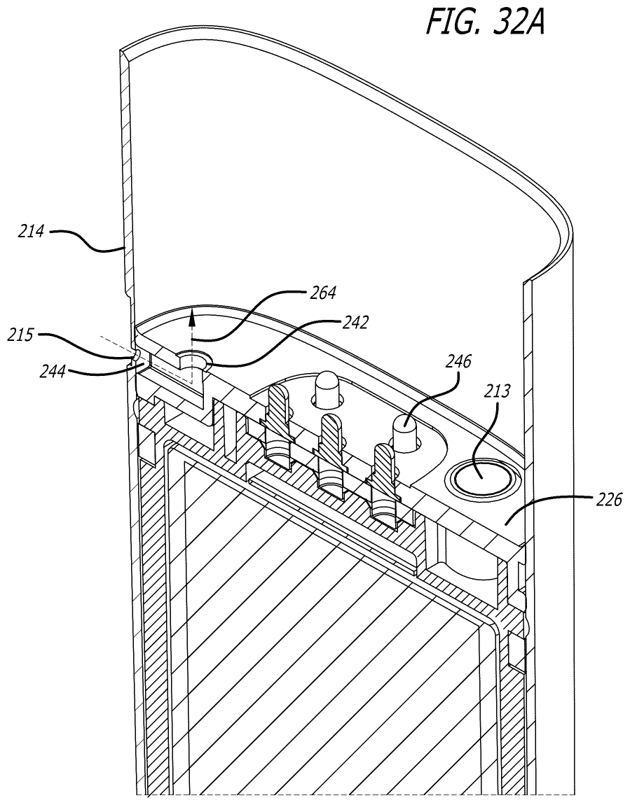

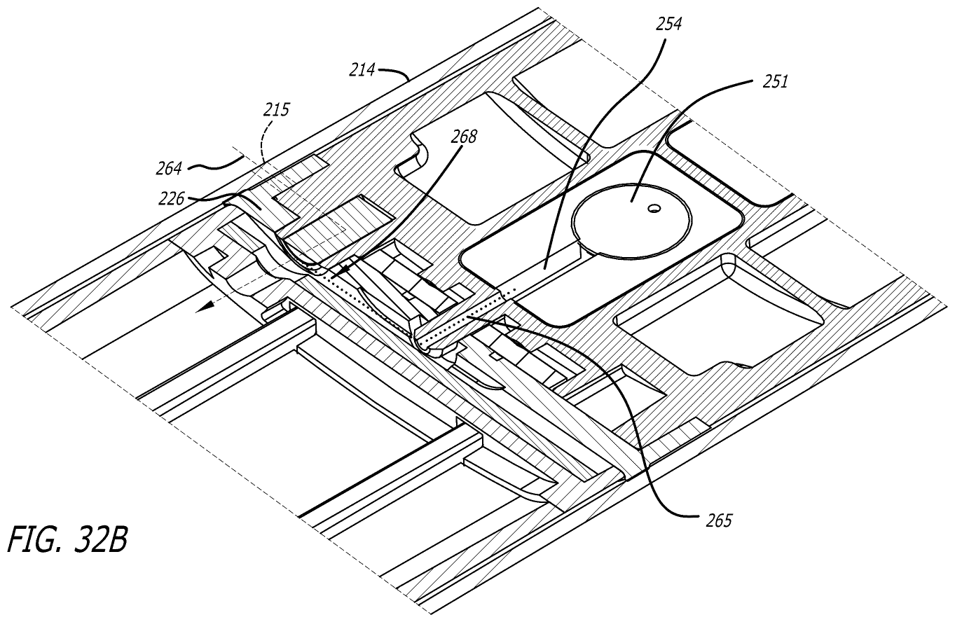

Once the driver 210 has been assembled, two air flow paths are formed-one for the intake of air that is routed through the bulkhead 226 to the pod 110, and another for allowing the air flow sensor 251 to determine a relative drop in air pressure due to the user drawing on the mouthpiece 112 of the pod 110. These air flow paths are shown in more detail in FIGS. 31, 32A, and 32B.

FIG. 31 shows the pod 110 spaced from the authentication PCB 212 and the bulkhead 226 for clarity. When the pod 110, authentication PCB 212, and bulkhead 226 are assembled, the transducer driving holes 221 may allow the drive signal connectors 247 to make contact with the electrical contacts 158 in the pod. Further, the retention pin receiving holes 224 may allow magnetic contact between the magnets 213 in the driver 210 and corresponding magnets 135 in the pod 110. Further still, the air passage 223 in the authentication PCB 212 permits the passage of air from the bulkhead intake conduit 245 to the air inlet hole 136 in the pod 110.

The depth of the cavity 211 in the open end 270 of the driver 210 and the length of the housing 111 of the pod 110 are preferably complimentary, such that once the pod 110 is received with in the cavity 211, the gap between mouthpiece 112 of the pod 110 and the driver casing 214 is minimal, or more preferably, non-existent, but a secure electrical contact between the pod and the signal connectors 246, 247 is made. Similarly, the thickness of the driver casing 214 is preferably complimentary to the thickness of the mouthpiece material. This gives the device a sleek finish, and also reduces the likelihood of dirt accumulation in a ridge between the mouthpiece 112 and the casing. Further, the risk of the pod 110 being inadvertently removed from the driver is minimised if there is no edge on either the casing 214 or the mouthpiece 112 to catch on a user's pocket or the like.

FIGS. 32A and 32B illustrate the air flow path of intake air and of the air flow sensor, in some examples, once the pod 110, authentication PCB 212, and bulkhead 226 are assembled. Note that some features of the pod 110 and driver 210 have been omitted for clarity. The intake air path, shown by the dashed lines and arrow 264, passes through the air intake aperture 215, the bulkhead intake conduit 245, the air passage 223 in the authentication PCB 212, and then into the air inlet hole 136 of the pod 110.

The air flow path of the air flow sensor is shown by dotted lines 265. The air flow path of the air flow sensor, in some examples, taps into the air intake flow path in the chamber 268 that may be formed between the bulkhead 226 and the authentication PCB 212. As air is drawn into the pod 110 by the user and the intake air flows into the pod 110, air is also drawn away from the air flow sensor 251 via the air flow sensor holder channel 254 and the air flow sensor tube 232. This creates the negative air pressure at the air flow sensor 251, which the air flow sensor 251 communicates to the main PCB 227, thereby allowing the ultrasonic transducer 123 to be activated to produce the mist.

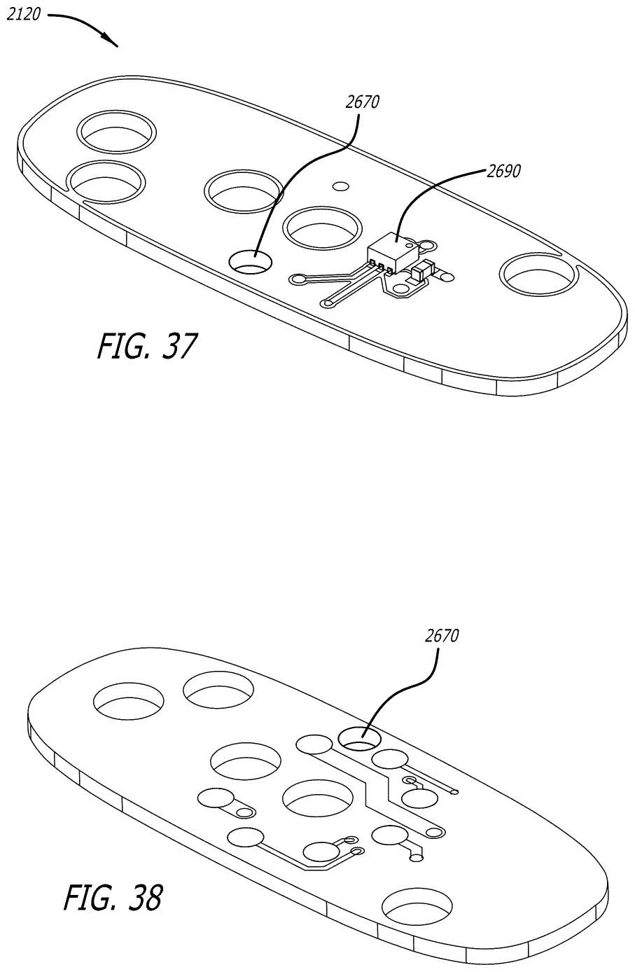

It will of course be appreciated that FIGS. 13 to 36 illustrate only one of many possible configurations of implementing the teachings of the disclosure herein. Other, but by no means all, example configurations and components are shown in FIGS. 37 to 42, and will be described in turn. Note that only differences relative to the above-described components may be highlighted for brevity.

FIGS. 37 and 38 illustrate an alternative authentication PCB 2120. In some examples, the authentication PCB 2120 may include a further through hole 2670. The further through hole 2670 may be positioned so as to allow the air flow sensor tube 2320 to pass therethrough. In some examples, the air sensor flow path may be between the authentication PCB 2120 and the end cap 113 of the pod 110.

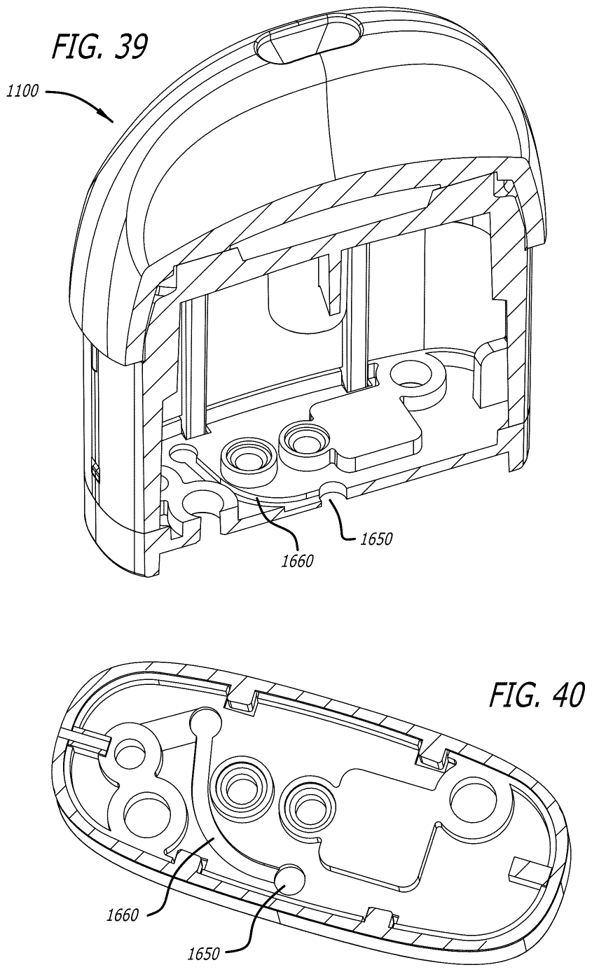

Alternatively, an example configuration such as that shown in FIGS. 39 to 42 may be implemented in combination with the extended air sensor flow tube 2320.

FIG. 39 shows a pod 1100 with an alternative end cap 1130, the alternative end cap 1130 shown in isolation in FIG. 40. The end cap 1130 includes an aperture 1650 and an arcuate duct 1660 to enable the air sensor flow path to tap into the air intake flow path between the end cap 1130 and the lower body portion 152 of the pod 1100. It should be noted that the duct 1660 need not be arcuate, and may take any shape, as required, to avoid other features of the end cap 1130. It is preferable that the duct 1660 is either straight or incorporates a minimal number of gradual turns, thereby providing a more efficient flow path including less turbulence.

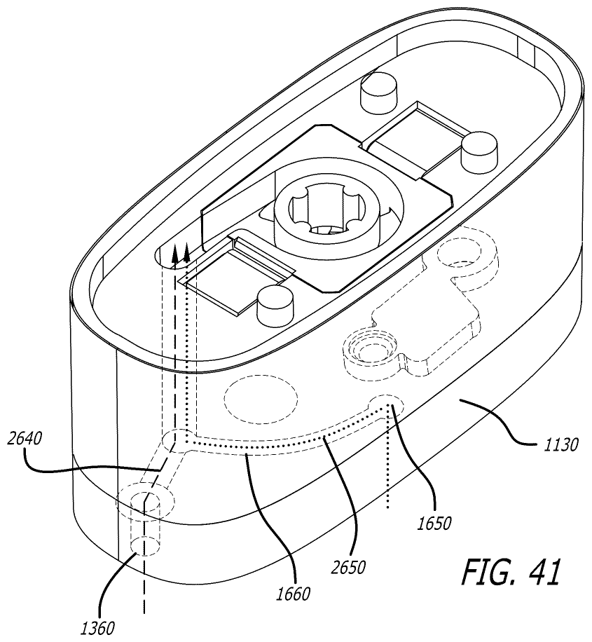

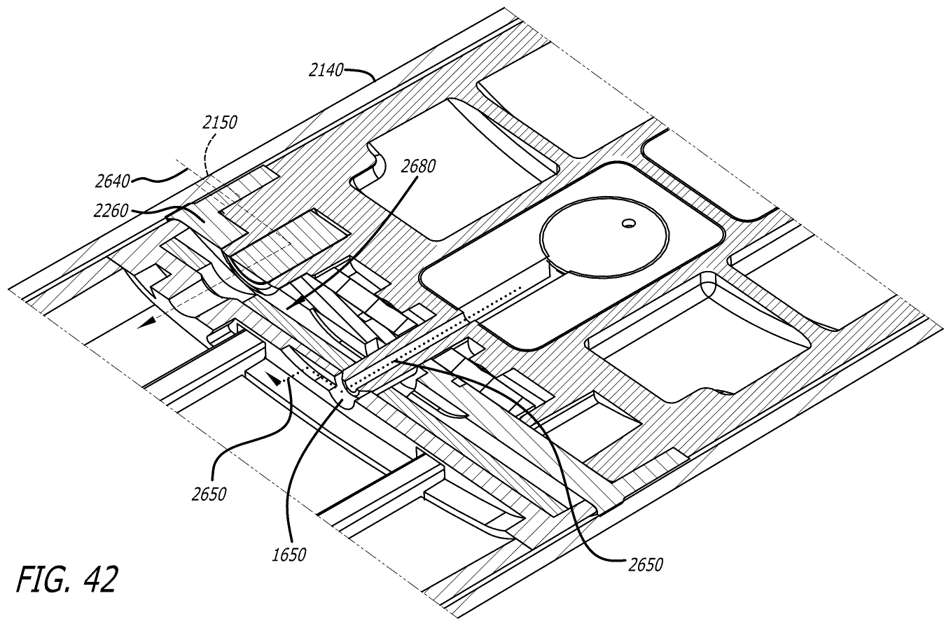

FIGS. 41 and 42 show the intake air flow path 2640, and the air sensor flow path 2650 tapping thereinto. It should be noted that the air sensor flow path in FIG. 42 illustrates the general direction of air flow, and not the specific path which the air may take. In this example, the air sensor flow path passes through the aperture 1650 in the end cap 1130 and then through the arcuate duct 1660. The intake air flow path 2640 and the air sensor flow path 2650 then join at the point in which the air intake flow path 2640 changes direction to travel to the spacer 121 and the manifold 122. The intake air flow path through the lower body portion 152 and the spacer 121 may represent the longest section of substantially straight flow path, and so tapping into this section may provide the most accurate readings to the air flow sensor due to lower turbulence and reduced eddy currents.

In this example, the bulkhead may not seal the components passing therethrough. Instead, the end cap 1130 may provide a seal for the air flow sensor tube 2320 and, in some embodiments, the bulkhead intake conduit 245. The intake air flow path can be similarly sealed.

The device is a compact, portable and highly advanced device that allows precise, safe and monitored aerosolisation. This is done by incorporating high-quality electronic components designed with IPC class 3—medical grade—in mind.

The electronics of the driver 210 are divided as such:

### 1. Sonication Section

In order to obtain the most efficient aerosolisation to date for inhalation in a portable device, with particle size below 1 μm, the sonication section has to provide the contacts pads receiving the ultrasonic transducer 123 (piezoelectrical ceramic disc (PZT)) with high adaptive frequency (approximately 3 MHz).

This section not only has to provide high frequency but also protect the ultrasonic transducer 123 against failures while providing constant optimised cavitation.

PZT mechanical deformation is linked to the AC Voltage amplitude that is applied to it, and in order to guarantee optimal functioning and delivery of the system at every sonication, the maximum deformation must be supplied to the PZT all the time.

However, in order to prevent the failure of the PZT, the active power transferred to it must be precisely controlled.

This could only be achieved by designing a custom, not existing in the market, Power Management Integrated Circuit (PMIC) chip which is provided on the printed circuit board of the driver 210. This PMIC allows modulation of the active power given to the PZT at every instant without compromising the mechanical amplitude of vibration of the PZT.

By Pulse Width Modulation (PWM) of the AC voltage applied to the PZT, the mechanical amplitude of the vibration remains the same.

The only ‘on the shelf’ option available would have been to modify the output AC voltage via the use of a Digital to Analog Converter (DAC). The energy transmitted to the PZT would be reduced but so would the mechanical deformation which as a result completely degrades and prevents proper aerosolisation. Indeed, the RMS voltage applied would be the same with effective Duty Cycle modulation as with Voltage modulation, but the active power transferred to the PZT would degrade. Indeed, given the formula below:

$$Active Power displayed to the PZT being Pa = \frac{2 \sqrt{2}}{π} Irms * Vrms * cos φ ,$$

Where
φ is the shift in phase between current and voltage
Iᵣₘₛ is the root mean square Current
Vᵣₘₛ is the root mean square Voltage.

When considering the first harmonic, Irms is a function of the real voltage amplitude applied to the transducer, as the pulse width modulation alters the duration of voltage supplied to the transducer, controlling Irms.

The specific design of the PMIC uses a state-of-the-art design, enabling ultra-precise control of the frequency range and steps to apply to the PZT including a complete set of feedback loops and monitoring path for the control section to use.

The rest of the aerosolisation section is composed of the DC/DC boost converter and transformer that carry the necessary power from a 3.7V battery to the PZT contact pads.

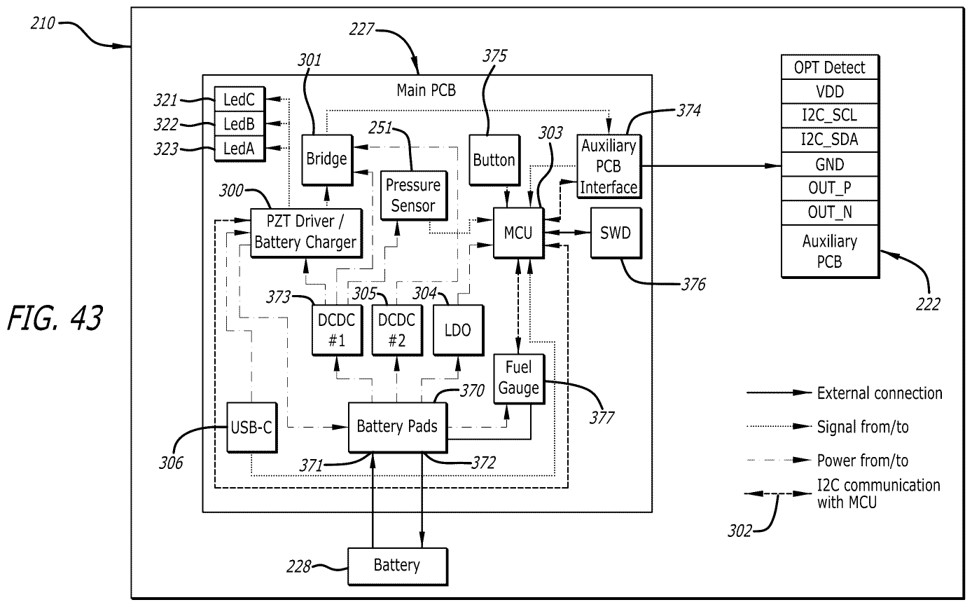

Referring now to FIG. 43 of the accompanying drawings, an apparatus for controlling a mist inhaler comprises the main PCB 227 and is provided within the driver 210. For simplicity, the following description will refer to the driver 210 as comprising elements of the apparatus.

The main PCB 227 includes a plurality of conductive tracks which provide an electrical connection between components mounted to the main PCB 227. The main PCB 227 comprises battery pads 370 that include a positive battery terminal 371 to connect to a positive terminal of the battery 228 and a ground battery terminal 372 to connect to a ground terminal of the battery 228.

The driver 210 comprises an ultrasonic transducer driver microchip which is referred to herein as a power management integrated circuit or PMIC 300. In some embodiments, the PMIC 300 comprises a battery charging sub-system. The PMIC 300 is a microchip for driving a resonant circuit. The resonant circuit is an inductance (L) capacitance (C) circuit (LC tank), an antenna or, in this case, a piezoelectric transducer (the ultrasonic transducer 123).

In this disclosure, the terms chip, microchip and integrated circuit are interchangeable. The microchip or integrated circuit is a single unit which comprises a plurality of interconnected embedded components and subsystems. The microchip is, for example, at least partly of a semiconductor, such as silicon, and is fabricated using semiconductor manufacturing techniques.

The driver 210 also comprises a second microchip which is referred to herein as a bridge integrated circuit or bridge IC 301 which is electrically connected to the PMIC 300. The bridge IC 301 is a microchip for driving a resonant circuit, such as an LC tank, an antenna or a piezoelectric transducer. The bridge IC 301 is a single unit which comprises a plurality of interconnected embedded components and subsystems.

In this example, the PMIC 300 and the bridge IC 301 are mounted to the same PCB 227 of the driver 210. In this example, the physical dimensions of the PMIC 300 are 1-3 mm wide and 1-3 mm long and the physical dimensions of the bridge IC 301 are 1-3 mm wide and 1-3 mm long.

The main PCB 227 comprises an auxiliary PCB interface 374 which is configured to communicate control signals from a controller 303 to the auxiliary PCB 222. The auxiliary PCB interface 374 which is also configured to communicate drive signals from the bridge IC 301 to the auxiliary PCB 222. The auxiliary PCB 222 the communicates the control signals and the drive signals to the pod 110 to control the operation of the pod 110.

The main PCB 227 is provided with a button input 375 which is connected electrically to the push button provided adjacent the base of the driver 210. The button input 375 receives a button signal whenever the button is pressed by a user. For example, a user may press the button (short press) to check the charge level of the battery 228. A user may press the button (long press of >3s) to cause the driver 210 to enter Bluetooth pairing mode to pair the driver 210 with an external computing device, such as smartphone.

The main PCB 227 may also be provided with a serial data port 376 which is connected electrically to the controller 303. The serial data port 376 may be used to communicate with the controller 303 either directly or via the I2C bus to control or program the driver 210.

When the pod 110 is coupled to the driver 210, the OTP IC 269 is electrically connected to the PMIC 300 to receive power from the PMIC 300 such that the PMIC 300 can manage the voltage supplied to the OTP IC 269. The OTP IC 269 is also connected to a communication bus 302 in the driver 210. In this example, the communication bus 302 is an I2C bus but in other examples the communication bus 302 is another type of digital serial communication bus.

The ultrasonic transducer 123 in the pod 110 is electrically connected to the bridge IC 301 via the auxiliary PCB 222 so that the ultrasonic transducer 123 may be driven by an AC drive signal generated by the bridge IC 301 when the device is in use.

The driver 210 comprises a controller or master control unit (MCU) in the form of a controller 303 which is electrically coupled for communication with the communication bus 302. The controller 303 is mounted to the main PCB 227 and connected electrically to receive power via the positive battery terminal 371 and the ground battery terminal 372. The controller 303 is preferably a computing device comprising a processor and a memory, the memory storing executable instructions which, when executed by the processor, control at least one function of the apparatus (driver 210). In some examples, the controller 303 is a Bluetooth™ low energy (BLE) controller. The controller 303 receives power from a low dropout regulator (LDO) 304 which is driven by the battery 228. The LDO 304 provides a stable regulated voltage to the controller 303 to enable the controller 303 to operate consistently even when there is a variation in the voltage of the battery 228.

The driver 210 comprises a voltage regulator in the form of a first DC-DC converter 373 which is connected electrically to the battery 228 via the battery pads 370. The first DC-DC converter 373 is a voltage regulator that provides a stable voltage to power the PMIC 300, control circuitry of the bridge IC 301 and the air flow sensor 251.

The driver 210 comprises a further voltage regulator in the form of a second DC-DC converter 305 which is connected electrically to the battery 228 via the battery pads 370. The second DC-DC converter 305 is a boost converter which increases the voltage of the battery 228 to a programmable voltage VBOOST. The second DC-DC converter 305 is configured to deliver a higher power than the first DC-DC converter 373.

The second DC-DC converter 305 forms part of a load driver circuit or main power trunk in the driver 210. The bridge IC 301 and the second DC-DC converter 305 together generate a load drive signal which may be used to drive a load, such as the ultrasonic transducer 123 in the pod 110.

The programmable voltage VBOOST is set by the second DC-DC converter 305 in response to a voltage control signal VCTL from the PMIC 300. As will be described in more detail below, the second DC-DC converter 305 outputs the voltage VBOOST to the bridge IC 301. In other examples, the voltage regulator is another type of voltage regulator which outputs a selectable voltage. The provision of two DC-DC converters 373, 305 enables power management within the driver 210 to be optimised by enabling the controller 303 to precisely control the voltages supplied to the electronic components. For example, the controller 303 can deactivate the second DC-DC converter 305 when there is no requirement for the higher voltage VBOOST (e.g. when inhalation is not occurring).

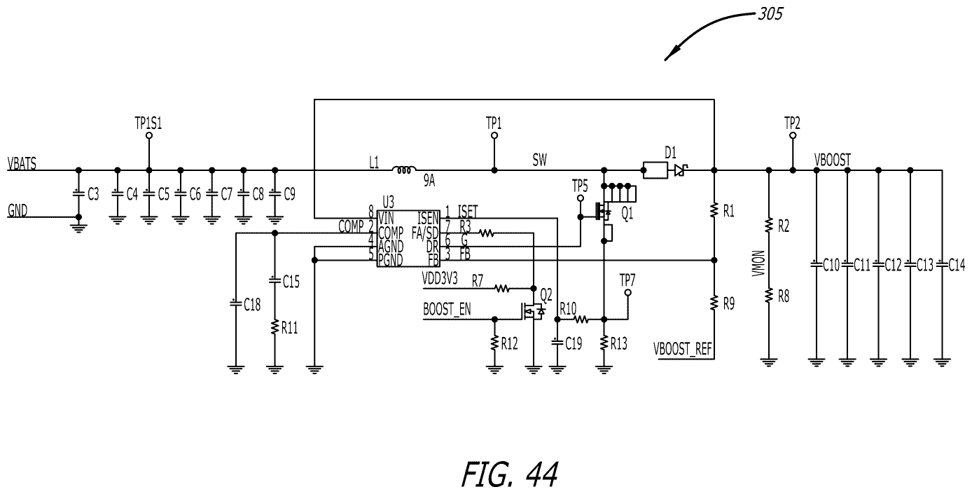

Referring now to FIG. 44 of the accompanying drawings, the second DC-DC converter 305 of some examples is a boost converter circuit that comprises an integrated circuit U3.

In some examples, the integrate circuit is of type LM3478MAX/NOPB. The boost converter circuit comprises a plurality of input capacitors C3-C9 that are connected in parallel between the positive battery supply voltage rail VBATS and ground GND. The boost converter circuit comprises an inductor L1, a diode D1, a switch Q1, a resistor R1 and a capacitor C19 that are connected at a boost converter circuit. The integrated circuit U3 controls the switch Q1 to switch on and off in sequence to boost the supply voltage from the battery VBATS to a higher boost voltage VBOOST (preferably approximately 24V) which is output to the bridge IC 301.

The boost converter circuit comprises output capacitors C10-C14 which filter and smooth the output voltage VBOOST for delivery to the load, which in this case is the bridge IC 301.

The voltage control signal VCTL is generated by a digital to analogue converter (DAC) which, in this example, is implemented within the PMIC 300. The DAC is not visible in FIG. 43 since the DAC is integrated within the PMIC 300. The DAC and the technical benefits of integrating the DAC within the PMIC 300 are described in detail below.

In this example, the PMIC 300 is connected to a power source connector in the form of a universal serial bus (USB) connector 306 so that the PMIC 300 can receive a charging voltage VCHRG when the USB connector 306 is coupled to a USB charger. The USB connector 306 is preferably a USB—C type connector. In other examples, the connector may be a different type of connector to USB-C while still being configured to receive a charging voltage VCHRG.

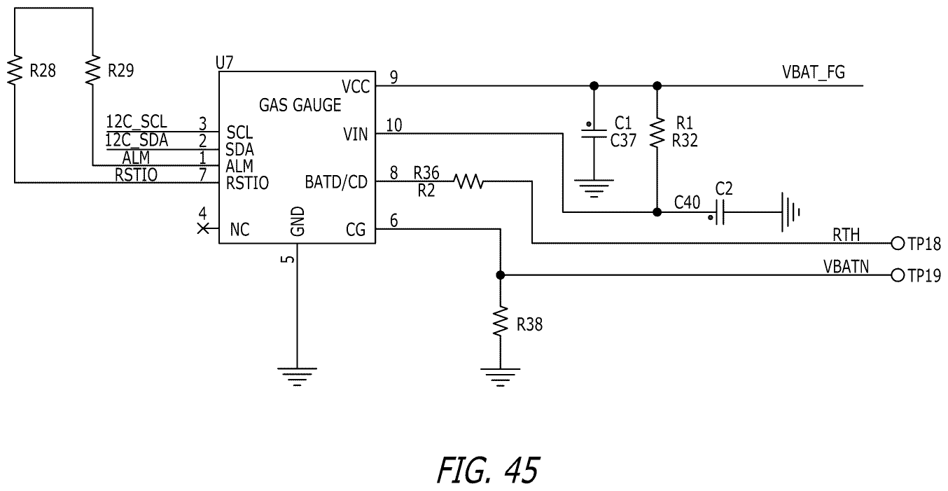

In some examples, the driver 210 comprises a fuel (gas) gauge 377 that is connected electrically to the positive and ground battery pads 371, 372. The fuel gauge 377 is configured to monitor the charge level of the battery 228 but monitoring the about of charge received from the battery 228 during operation and the charge provided to the battery 228 during charging. FIG. 45 of the accompanying drawings shows an example circuit diagram for the fuel gauge 377. The fuel gauge 377 comprises an integrated circuit U7 that is configured to communicate a charge signal to the controller 303 which is indicative of the charge level of the battery 228. In some examples, the integrated circuit U7 is of type STC3115.

The fuel gauge 377 is powered by a voltage applied at VBAT_FG. The fuel gauge 377 senses the current flowing through the shunt resistor R38 which is coupled between the negative battery terminal 372 and the ground plane of the main PCB 227. The shunt resistor R38 is preferably 5 m² to 50 mΩ. The accuracy of the value of the shunt resistor R38 is preferably 2% or better.

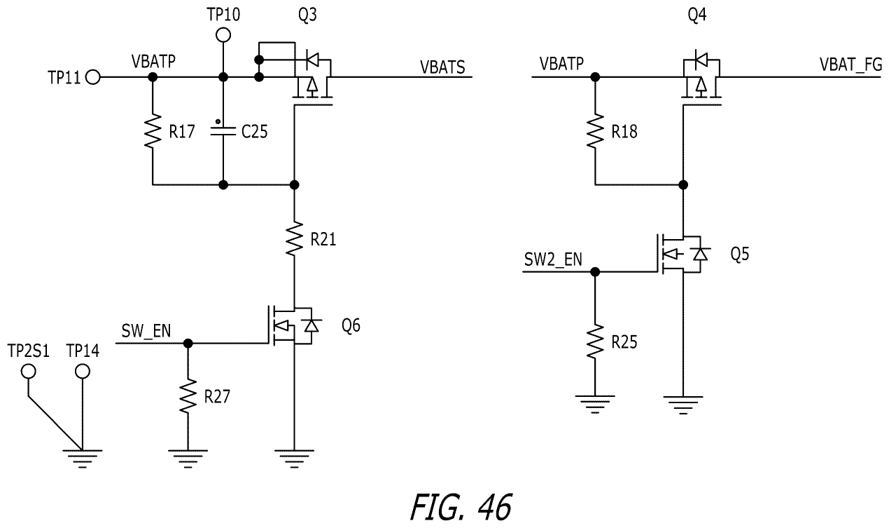

Referring now to FIG. 46 of the accompanying drawings, in some examples an apparatus within the driver 210 comprises a first switch Q3 connected between the positive battery terminal 371 (VBATP) and the load driver circuit (main trunk circuit and/or the second DC-DC converter circuit 305) at VBATS. The first switch Q3 may be a high side switch. The first switch Q3 is controllable by the controller 303 to electrically connect the load driver circuit (VBATS) to the positive battery terminal 371 when the load driver circuit (incorporating at least the second DC-DC converter circuit 305) is in use and to electrically disconnect the load driver circuit from the positive battery terminal 371 when the load driver circuit is not in use. The first switch Q3 can therefore connect/disconnect the entire power trunk to and from the battery 228 to guarantee zero power consumption by the power trunk when the first switch Q3 is off. This minimises unnecessary power consumption by the battery 228 and thereby optimises battery life.

The apparatus may comprise capacitor C25 and resistor R17 that are connected to the switch Q3 to provide current inrush protection so that the first switch Q3 turns on in a well-defined slope.

The first switch Q3 is preferably controlled by a first control switch Q6 which is turned on and off by a control signal (SW_EN) from the controller 303.

In some examples an apparatus within the driver 210 comprises a second switch Q4 which is connected between the positive battery terminal 371 (VBATP) and the fuel gauge circuit. The second switch Q4 may be a high side switch. The second switch Q4 is controllable by the controller 303 to electrically connect the fuel gauge circuit (VBAT_FG) to the positive battery terminal 371 when the fuel gauge circuit is in use and to electrically disconnect the fuel gauge circuit from the positive battery terminal 371 when the fuel gauge circuit is not in use. This minimises unnecessary power consumption by the battery 228 and thereby optimises battery life.

The second switch Q4 is preferably controlled by a second control switch Q5 which is turned on and off by a second control signal (SW2_EN) from the controller 303.

It is to be appreciated that the controller 303 remains connected electrically to receive power via the positive battery terminal 371 and the ground battery terminal 372 to control at least one function of the apparatus even when the load driver circuit and/or the fuel gauge circuit are disconnected.

In some examples, the second switch Q4 is optional and is omitted and the apparatus is not configured to disconnect the fuel gauge circuit when the fuel gauge circuit is not in use. In other examples, the first switch Q3 is optional and is omitted and the apparatus is not configured to disconnect the load driver circuit when the load driver circuit is not in use.

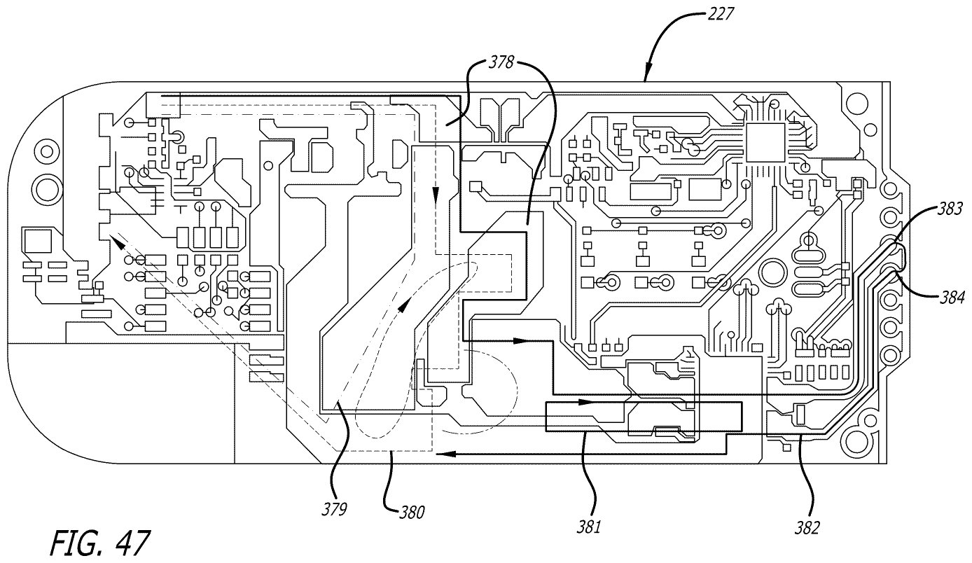

Referring now to FIG. 47 of the accompanying drawings, the layout of the main PCB 227 may be optimised by the arrangement of the plurality of conductive tracks 378 and the position of the components on the main PCB 227. The layout of the main PCB 227 is optimised for most-efficient-path for the driver power signals to the interface with the pod 110 (at the auxiliary PCB 222). The plurality of conductive tracks 378 and the position of the components on the main PCB 227 may be arranged to conduct currents across the main PCB 227 in a plurality of current loops 379-381 for delivering current at different current levels to circuits and sub-systems. In this example, the main PCB 227 is configured to deliver current at different current levels to circuits and sub-systems across four current loops 379-381. For example, to deliver a higher current in a loop 381 to transducer output pins 383 (OUT_P) and 384 (OUT_N).

First current loop 379:
a. Loop 1 from the positive terminal 371 of the battery 228 through the inductor L1 to a low-side-switch back through the shunt resistor R38 to the negative terminal 372 of the battery227.
b. Loop 1* from the positive terminal 371 of the battery 228 to the input capacitances C3-C9 and back through the shunt resistor R38 to the negative terminal 372 of the battery 228.
   Second current loop 380:
Loop 2 from the input capacitance C3-C9 through the inductor L1 to the low-side-switch back through the shunt resistor R38 to the negative terminal 372 of the battery 228.
   Third current loop 381:
Loop 3 to charge the output capacitors C10-C14.
   Third current loop 382:
Loop 4 is the loop where energy is transferred from the output capacitors C10-C14 to the load. In this case, the load is connected to the bridge IC 301 which directs the current in one half cycle of a 3 MHz period to the load (ultrasonic transducer 123 in the pod 110 via the transducer output pins 383 (OUT_P) and 384 (OUT_N)) and in the second half cycle in counter polarity to the load.

The layout of the main PCB 227 is further optimised by the conductive tracks 378 being routed, preferably on the top level only of the main PCB 227, to minimise impedance in the conductive tracks 378. This low impedance routing improves the operation of the driver 210 by enabling signals to be conducted across the main PCB 227 at high speeds with minimal distortion/interference.

The conductive tracks 378 are preferably laid out as wide as possible relative to one another on the main PCB 227. The main PCB 227 comprises a well-defined return path at a star connected ground (GND) node on a bottom edge of the PCB (connecting to the input capacitors, low side FET S-terminal, output capacitance and Bridge GND connection).

As described herein, the main PCB 227 comprises an H bridge circuit implemented in the bridge IC 301. The H bridge circuit has two AC outputs which are electrically connected to first ends of two respective AC conductive tracks of the plurality of conductive tracks 378 of the main PCB, the two AC conductive tracks being positioned proximate to one another and terminate at the output pins 383 (OUT_P) and 384 (OUT_N). The conductive tracks 378 to the differential output pins 383 (OUT_P) and 384 (OUT_N) are routed as close as possible to one another. This minimises the magnetic field as much as possible and reduces the inductance to the ultrasonic transducer 123 in the pod 110. Power transfer to the ultrasonic transducer 123 is thus maximised and battery life of the driver 210 is optimised.

The main PCB 227 comprises a conductive ground plane and has a load driver circuit ground terminal which is connected electrically to the ground plane. The ground plane (bottom metal layer of the main PCB 227) extends across a majority of the one side of the main PCB 227. For example, the ground plane may cover and/or extend across 70% to 90% or more of one side of the main PCB 227. The shunt resistor R38 is connected electrically between the ground plane of the main PCB and the ground battery terminal and the shunt resistor R38 is preferably positioned on the underside of the main PCB 227 (the same side of the main PCB 227 as the ground plane). This configuration enhances the robustness of the ground plane.

In some examples, the ground plane (GND node) is not the most negative node in the apparatus/driver 210 but the battery negative terminal 372 (BATN) is the most negative node. The shunt resistor R38 is therefore connected between the ground plane (GND) and the battery negative terminal 372 (BATN) to be able to measure the battery current. Substantially the entire return current flows through the ground plane bottom layer metal through the shunt resistor R38 back to the negative battery terminal 372.

The battery return current can reach magnitudes of up to 10 A. To avoid changing layers and directing the current through vias (current crowding) the shunt resistor R38 may be placed on the bottom side of the PCB where the (huge) ground plane (GND) plane forms the current return path. After this, the current does not change layers on the main PCB 227 any further but keeps flowing from the ground plane, funnelling through the shunt resistor R38, to the negative battery terminal 372.

The air flow sensor 251 is mounted to the main PCB 227. In some examples, the air flow sensor 251 is a static pressure sensor. In some examples, the driver 210 may comprise a second air flow sensor which may take the form of a dynamic pressure sensor. As described above, the air flow sensor 251 senses a change in the pressure in the sonication chamber 142 to sense inhalation when a user is drawing on the mouthpiece 112.

The driver 210 comprises three LEDs 321-323 which are mounted to the main PCB 227. The LEDs 321-3232 are preferably received in the recesses 257 of the air flow sensor cover 229. The LEDs 321-323 are controlled by the PMIC 300. In some examples, the driver 210 comprises a greater or few number of LEDs and in other examples the LEDs may be omitted entirely.

The controller 303 functions as a master device on the communication bus 302, with the PMIC 300 being a first slave device. The OTP IC 269 and the air flow sensor 251 may also be slave devices. The communication bus 302 enables the controller 303 to control the following functions within the driver 210:
1. All functions of the PMIC are highly configurable by the controller 303.
2. The current flowing through the ultrasonic transducer 123 is sensed by a high bandwidth sense and rectifier circuit at a high common mode voltage (high side of the bridge). The sensed current is converted into a voltage proportional to the rms current and provided as a buffered voltage at a current sense output pin 309 of the bridge IC 301. This voltage is fed to and sampled in the PMIC 300 and made available as a digital representation via I2C requests. Sensing the current flowing through the ultrasonic transducer 123 forms part of the resonant frequency tracking functionality. As described herein, the ability of the device to enable this functionality within the bridge IC 301 provides significant technical benefits.
3. The DAC (not shown in FIG. 43) integrated within the PMIC 300 enables the DC-DC converter voltage VBOOST to be programmed to be between 10V and 20V.
4. The controller 303 enables the charger sub-system of the driver 210 to manage the charging of the battery 228, which in this example is a single cell battery.
5. A Light Emitting Diode (LED) driver module (not shown) is powered by the PMIC 300 to drive and dim digitally the LEDs 321-323 either in linear mode or in gamma corrected mode.
6. The controller 303 is able to read pressure sensor values from the air flow sensor 251.

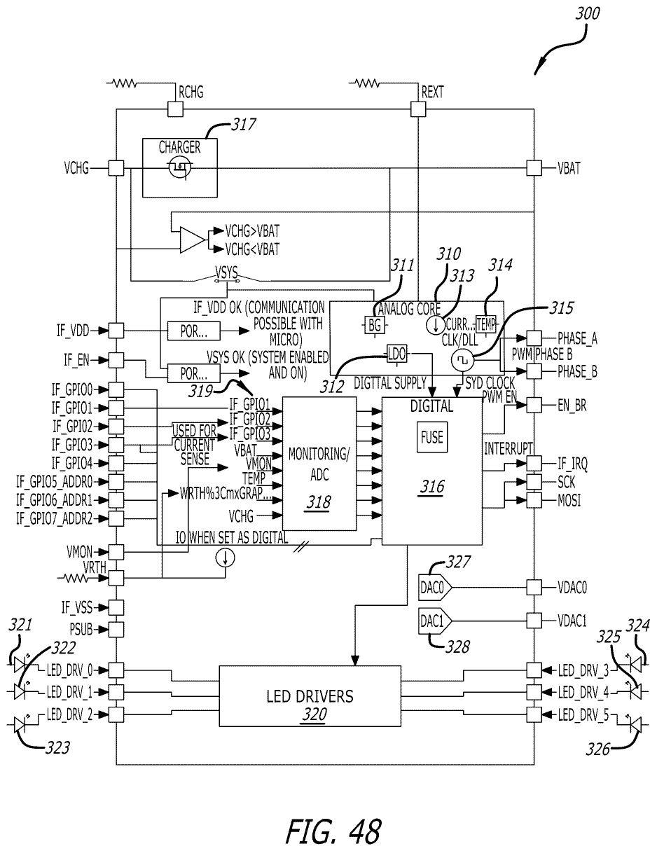

Referring now to FIG. 48 of the accompanying drawings, the PMIC 300 is, in this example, a self-contained chip or integrated circuit which comprises integrated subsystems and a plurality of pins which provide electrical inputs and outputs to the PMIC 300. The references to an integrated circuit or chip in this disclosure are interchangeable and either term encompasses a semiconductor device which may, for instance, be of silicon.

The PMIC 300 comprises an analogue core 310 which comprises analogue components including a reference block (BG) 311, a LDO 312, a current sensor 313, a temperature sensor 314 and an oscillator 315.

As described in more detail below, the oscillator 315 is coupled to a delay locked loop (DLL) which outputs pulse width modulation (PWM) phases A and B. The oscillator 315 and the DLL generate a two phase centre aligned PWM output which drives an H bridge in the bridge IC 301.

The DLL comprises a plurality of delay lines connected end to end, wherein the total delay of the delay lines is equal to the period of the main clock signal clk_m. In this example, the DLL is implemented in a digital processor subsystem, referred to herein as a digital core 316, of the PMIC 300 which receives a clock signal from the oscillator 315 and a regulated power supply voltage from the LDO 312. The DLL is implemented in a large number (e.g. in the order of millions) of delay gates which are connected end to end in the digital core 316.

The implementation of the oscillator 315 and the DLL in the same integrated circuit of the PMIC 300 in order to generate a two phase centre aligned PWM signal is unique since at present no signal generator component in the integrated circuit market comprises this implementation.

As described herein, PWM is part of the functionality which enables the driver 210 to track the resonant frequency of the ultrasonic transducer 123 accurately in order to maintain an efficient transfer from electrical energy to kinetic energy in order to optimise the generation of mist.

In this example, the PMIC 300 comprises a charger circuit 317 which controls the charging of the battery 228, for instance by power from a USB power source.

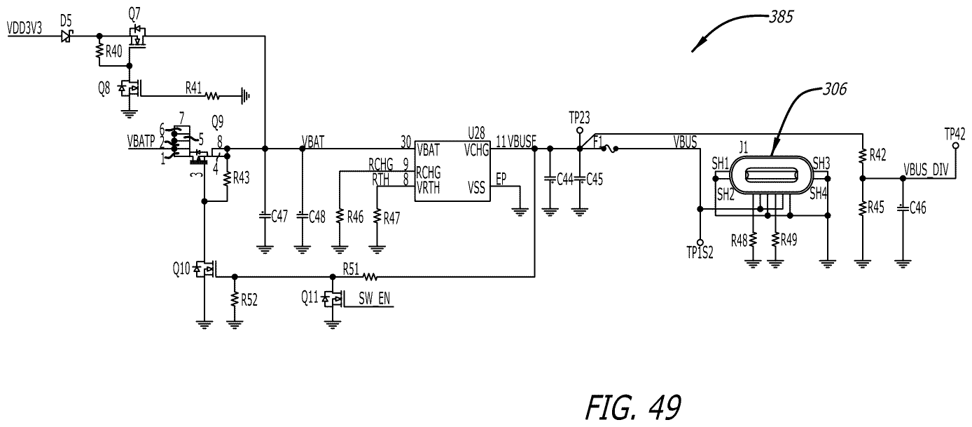

Referring now to FIG. 49 of the accompanying drawings, a charging control sub-system 385 (or power control circuit) of some examples is connected electrically to the USB port 306 to receive a voltage VBUS from an external power source connected to the USB port 306. The charging control sub-system 385 is also connected electrically to the positive battery pad 371. In some examples, the charging control sub-system 385 controls the charging of the battery 228 with power supplied via the USB port 306. The charging control sub-system 385 is controlled by the controller 303 to prevent current from being drawn from the USB port 306 when the driver 210 is activated drive the pod 110 to generate a mist.

The charging control sub-system 385 comprises a switch Q9 that is connected between a terminal VBAT of the charging control sub-system 385 and the positive battery pad 371. The switch Q9 is controllable by the controller 303 to switch on to connect the positive battery terminal 371 to the charging control sub-system 385 (power control circuit) and to switch off to disconnect the positive battery terminal 371 from the charging control sub-system 385.

The switch Q9 is a MOSFET which incorporates an inherent body diode between the source and drain terminals of the MOSFET. The switch Q9 is oriented so that the voltage of the battery 228 is present at the VBAT terminal even when switch Q9 is off. The voltage at the VBAT terminal is reduced in this condition due to the forward voltage drop across the body diode but the voltage is still detectable to enable the voltage of the battery 228 to be monitored.

The charging control sub-system 385 comprises a switch Q10 connected to the power supply input terminal 306 (USB port). The switch Q10 is configured to turn on the switch Q9 when power is received at the power supply input terminal 306 so that the power can charge the battery 228. The controller 303 is configured to turn the switch Q10 off in response to the controller 303 receiving a signal indicative of an inhalation by a user so that power is drawn by the load driver circuit from the battery 228 and not from the external power supply during inhalation. This improves the safety of the driver 210 by minimising excess current draw from an external power source. The driver 210 operation is optimised by ensuring that a regulated and sufficient current is delivered by the battery 228 during inhalation. The driver 210 can thus drive the pod 110 to deliver a mist consistently without the operation being affected by the capability of an external power source (which may deliver insufficient, inconsistent or unreliable power).

The charging control sub-system 385 enables the driver 210 to function in the selected modes for the following cases:
1) The battery is completely depleted, and the charging control sub-system 385 needs to charge the battery 228 in standalone mode: If an external power source is connected to the USB port 306 to apply a voltage, VBUS, the voltage VBUS turns ON switch Q10 which turns ON switch Q9. Switch Q9 forms a low impedance path from the charging control sub-system 378 to the battery 228. The battery 228 can therefore charge.
2) The battery is charging (in normal mode) and user wants to inhale (causing the driver 210 to activate to drive the pod 110 to generate a mist): Since charging and inhaling is prohibited but inhaling/activation has priority, charging is stopped through switch Q11 (turning Q10 OFF by pulling the gate down and subsequently Q9 as well) as long as the DC-DC main power trunk (load driver circuit) is activated by SW_EN. In this case it is guaranteed that the battery is not charged during inhalation by a user. This improves the safety of the driver by ensuring that charging of the battery does not occur during inhalation. Damage to the driver and/or damage to an external power source connected to the device is thereby prevented.
3) The battery is not charging (external power source not connected to driver) and the user starts inhalation: Q9 is switched OFF since VBUS is zero to avoid low battery voltages or tanking battery voltages triggering a power-on-reset (POR) from the PMIC 300. Triggering a POR would lead to a shutdown of the DAC output of the PMIC 300 which has consequences on the DC-DC power trunk loop (load driver circuit). Q9 bulk/body diode still provides a voltage to the PMIC 300 (but the reverse path of Q9 is switched off). The main current support during the inhalation is delivered through Q7 which connects the power supply voltage (3.3V) from the first (small) DC-DC converter 373 to the VBAT pin by asserting a VBAT_SUP signal. The diode D5 prevents current flow from the battery (in case Q9 is ON) to the output of the first (small) DC-DC converter 373.

The PMIC 300 comprises an integrated power switch VSYS which configures the PMIC 300 to power the analogue core 310 by power from the battery 228 or by power from an external power source if the battery 228 is being charged.

The PMIC 300 comprises an embedded analogue to digital converter (ADC) subsystem 318. The implementation of the ADC 318 together with the oscillator 315 in the same integrated circuit is, in itself, unique since there is no other integrated circuit in the integrated circuit market which comprises an oscillator and an ADC implemented as sub-blocks within the integrated circuit. In a conventional device, an ADC is typically provided as a separate discrete component from an oscillator with the separate ADC and oscillator being mounted to the same PCB. The problem with this conventional arrangement is that the two separate components of the ADC and the oscillator take up space unnecessarily on the PCB. A further problem is that the conventional ADC and oscillator are usually connected to one another by a serial data communication bus, such as an I2C bus, which has a limited communication speed of up to only 400 kHz. In contrast to conventional devices, the PMIC 300 comprises the ADC 318 and the oscillator 315 integrated within the same integrated circuit which eliminates any lag in communication between the ADC 318 and the oscillator 315, meaning that the ADC 318 and the oscillator 315 can communicate with one another at high speed, such as at the speed of the oscillator 315 (e.g. 3 MHz to 5 MHz).

In the PMIC 300 of this example, the oscillator 315 is running at 5 MHz and generates a clock signal SYS CLOCK at 5 MHz. However, in other examples, the oscillator 315 may generate a clock signal at a much higher frequency, such as up to 105 MHz. The integrated circuits described herein are all configured to operate at the high frequency of the oscillator 315.

The ADC 318 comprises a plurality of feedback input terminals or analogue inputs 319 which comprise a plurality of GPIO inputs (IF_GPIO1-3). At least one of the feedback input terminals or the analogue inputs 319 receives a feedback signal from an H-bridge circuit in the bridge IC 301, the feedback signal being indicative of a parameter of the operation of the H-bridge circuit or an AC drive signal when the H-bridge circuit is driving a resonant circuit, such as the ultrasonic transducer 123, with the AC drive signal. As described below, the GPIO inputs are used to receive a current sense signal from the bridge IC 301 which is indicative of the route mean square (rms) current reported by the bridge IC 301. In this example, one of the GPIO inputs is a feedback input terminal which receives a feedback signal from the H-bridge in the bridge IC 301.

The ADC subsystem 318 samples analogue signals received at the plurality of ADC input terminals 319 at a sampling frequency which is proportional to the frequency of the main clock signal. The ADC subsystem 318 then generates ADC digital signals using the sampled analogue signals.

In this example, the ADC 318 which is incorporated in the PMIC 300 samples not only the RMS current flowing through the H-bridge 334 and the ultrasonic transducer 123 but also voltages available in the system (e.g. VBAT, VCHRG, VBOOST), the temperature of the PMIC 300, the temperature of the battery 228 and the GPIO inputs (IF_GPIO1-3) which allow for future extensions.

The digital core 316 receives the ADC generated digital signals from the ADC subsystem and processes the ADC digital signals to generate the driver control signal. The digital core 316 communicates the driver control signal to the PWM signal generator subsystem (DLL 332) to control the PWM signal generator subsystem.

Rectification circuits existing in the market today have a very limited bandwidth (typically less than 1 MHz). Since the oscillator 315 of the PMIC 300 is running at up to 5 MHz or even up to 105 Mhz, a high bandwidth rectifier circuit is implemented in the PMIC 300. As will be described below, sensing the RMS current within an H bridge of the bridge IC 301 forms part of a feedback loop which enables the driver 210 to drive the ultrasonic transducer 123 with high precision. The feedback loop is a game changer in the industry of driving ultrasound transducers since it accommodates for any process variation in the piezo electric transducer production (variations of resonance frequencies) and it compensates for temperature effects of the resonance frequency. This is achieved, in part, by the inventive realisation of integrating the ADC 318, the oscillator 315 and the DLL within the same integrated circuit of the PMIC 300. The integration enables these sub-systems to communicate with one another at high speed (e.g. at the clock frequency of 5 MHz or up to 105 MHz). Reducing the lag between these subsystems is a game changer in the ultrasonics industry, particularly in the field of mist inhaler devices.

The ADC 318 comprises a battery voltage monitoring input VBAT and a charger input voltage monitoring input VCHG as well as voltage monitoring inputs VMON and VRTH as well as a temperature monitoring input TEMP.

The temperature monitoring input TEMP receives a temperature signal from the temperature sensor 314 which is embedded within the PMIC 300. This enables the PMIC 300 to sense the actual temperature within the PMIC 300 accurately so that the PMIC 300 can detect any malfunction within the PMIC 300 as well as malfunction to other components on the printed circuit board which affect the temperature of the PMIC 300. The PMIC 300 can then control the bridge IC 301 to prevent excitation of the ultrasonic transducer 123 if there is a malfunction in order to maintain the safety of the driver 210.

The additional temperature sensor input VRTH receives a temperature sensing signal from an external temperature sensor within the driver 210 which monitors the temperature of the battery 228. The PMIC 300 can thus react to stop the battery 228 from being charged in the event of a high battery temperature or otherwise shut down the driver 210 in order to reduce the risk of damage being caused by an excessively high battery temperature.

The PMIC 300 comprises an LED driver 320 which receives a digital drive signal from the digital core 316 and provides LED drive output signals to the LEDs 321-323 which are configured to be coupled to output pins of the PMIC 300. The LED driver 320 is configured to drive up to six LEDs 321-326 but in the examples described herein that comprise three LEDs 321-323, the LED driver 320 drives only those three LEDs 321-323. The LED driver 320 is preferably configured to drive and dim LEDs 321-326 in up to six independent channels.

The PMIC 300 comprises a first digital to analogue converter (DAC) 327 which converts digital signals within the PMIC 300 into an analogue voltage control signal which is output from the PMIC 300 via an output pin VDAC0. The first DAC 327 converts a digital control signal generated by the digital core 316 into an analogue voltage control signal which is output via the output pin VDAC0 to control a voltage regulator circuit, such as the second DC-DC converter 305. The voltage control signal thus controls the voltage regulator circuit to generate a predetermined voltage for modulation by the H-bridge circuit to drive a resonant circuit, such as the ultrasonic transducer 123, in response to feedback signals which are indicative of the operation of the resonant circuit (the ultrasonic transducer 123).

In this example, the PMIC 300 comprises a second DAC 328 which converts digital signals within the PMIC 300 into an analogue signal which is output from the PMIC 300 via a second analogue output pin VDAC1.

Embedding the DACs 327, 328 within the same microchip as the other subsystems of the PMIC 300 allows the DACs 327, 328 to communicate with the digital core 316 and other components within the PMIC 300 at high speed with no or minimal communication lag. The DACs 327, 328 provide analogue outputs which control external feedback loops. For instance, the first DAC 327 provides the control signal VCTL to the second DC-DC converter 305 to control the operation of the second DC-DC converter 305. In other examples, the DACs 327, 328 are configured to provide a drive signal to a DC-DC buck converter instead of or in addition to the second DC-DC converter 305. Integrating the two independent DAC channels in the PMIC 300 enables the PMIC 300 to manipulate the feedback loop of any regulator used in the driver 210 and allows the driver 210 to regulate the sonication power of the ultrasonic transducer 123 or to set analogue thresholds for absolute maximum current and temperature settings of the ultrasonic transducer 123.

The PMIC 300 comprises a serial communication interface 376 which, in this example, is an I2C interface which incorporates external I2C address set through pins.

The PMIC 300 also comprises various functional blocks which include a digital machine (FSM) to implement the functionality of the microchip. These blocks will be described in more detail below.

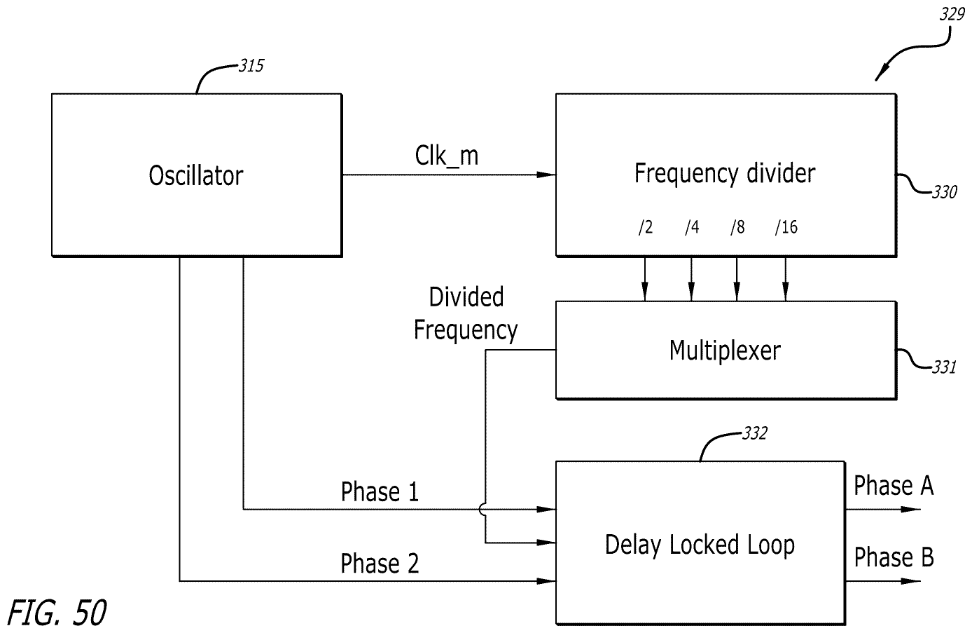

Referring now to FIG. 50 of the accompanying drawings, a pulse width modulation (PWM) signal generator subsystem 329 is embedded within the PMIC 300. The PWM generator system 329 comprises the oscillator 315, and frequency divider 330, a multiplexer 331 and a delay locked loop (DLL) 332. As will be described below, the PWM generator system 329 is a two phase centre aligned PWM generator.

The frequency divider 330, the multiplexer 331 and the DLL 332 are implemented in digital logic components (e.g. transistors, logic gates, etc.) within the digital core 316.

In examples of this disclosure, the frequency range which is covered by the oscillator 315 and respectively by the PWM generator system 329 is 50 KHz to 5 MHz or up to 105 MHz.

The frequency accuracy of the PWM generator system 329 is ±1% and the spread over temperature is ±1%. In the IC market today, no IC has an embedded oscillator and two phase centre aligned PWM generator that can provide a frequency range of 50 KHz to 5 MHz or up to 105 MHz.

The oscillator 315 generates a main clock signal (clk_m) with a frequency of 50 kHz to 5 MHz or up to 105 MHz. The main clock clk_m is input to the frequency divider 330 which divides the frequency of the main clock clk_m by one or more predetermined divisor amounts. In this example, the frequency divider 330 divides the frequency of the main clock clk_m by 2, 4, 8 and 16 and provides the divided frequency clocks as outputs to the multiplexer 331. The multiplexer 331 multiplexes the divided frequency clocks and provides a divided frequency output to the DLL 332. This signal which is passed to the DLL 332 is a frequency reference signal which controls the DLL 332 to output signals at a desired frequency. In other examples, the frequency divider 330 and the multiplexer 331 are omitted.

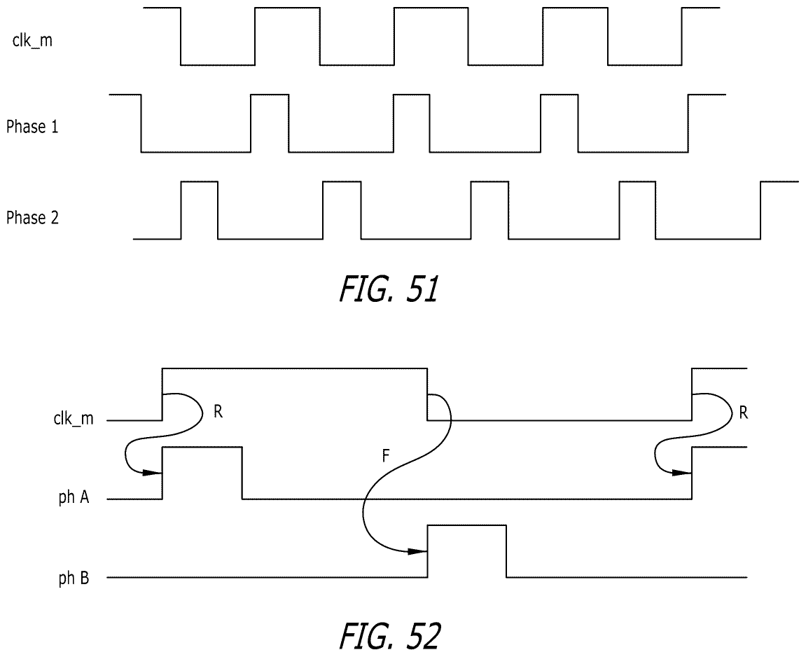

The oscillator 315 also generates two phases; a first phase clock signal Phase 1 and a second phase clock signal Phase 2. The phases of the first phase clock signal and the second phase clock signal are centre aligned. As illustrated in FIG. 51:
The first phase clock signal Phase 1 is high for a variable time of clk_m's positive half-period and low during clk_m's negative half-period.
The second phase clock signal Phase 2 is high for a variable time of clk_m's negative half-period and low during clk_m's positive half-period.

Phase 1 and Phase 2 are then sent to the DLL 332 which generates a double frequency clock signal using the first phase clock signal Phase 1 and the second phase clock signal Phase 2. The double frequency clock signal is double the frequency of the main clock signal clk_m. In this example, an “OR” gate within the DLL 332 generates the double frequency clock signal using the first phase clock signal Phase 1 and the second phase clock signal Phase 2. This double frequency clock or the divided frequency coming from the frequency divider 330 is selected based on a target frequency selected and then used as reference for the DLL 332.

Within the DLL 332, a signal referred to hereafter as “clock” represents the main clock clk_m multiplied by 2, while a signal referred to hereafter as “clock_del” is a replica of clock delayed by one period of the frequency. Clock and clock_del are passed through a phase frequency detector. A node Vc is then charged or discharged by a charge-pump based on the phase error polarity. A control voltage is fed directly to control the delay of every single delay unit within the DLL 332 until the total delay of the DLL 332 is exactly one period.

The DLL 332 controls the rising edge of the first phase clock signal Phase 1 and the second phase clock signal Phase 2 to be synchronous with the rising edge of the double frequency clock signal. The DLL 332 adjusts the frequency and the duty cycle of the first phase clock signal Phase 1 and the second phase clock signal Phase 2 in response to a respective frequency reference signal and a duty cycle control signal to produce a first phase output signal Phase A and a second phase output signal Phase B to drive an H-bridge or an inverter to generate an AC drive signal to drive an ultrasonic transducer.

The PMIC 300 comprises a first phase output signal terminal PHASE_A which outputs the first phase output signal Phase A to an H-bridge circuit and a second phase output signal terminal PHASE_B which outputs the second phase output signal Phase B to an H-bridge circuit.

In this example, the DLL 332 adjusts the duty cycle of the first phase clock signal Phase 1 and the second phase clock signal Phase 2 in response to the duty cycle control signal by varying the delay of each delay line in the DLL 332 response to the duty cycle control signal.

The clock is used at double of its frequency because guarantees better accuracy. As shown in FIG. 52, for the purpose of explanation if the frequency of the main clock clk_m is used (which it is not in examples of this disclosure), Phase A is synchronous with clock's rising edge R, while Phase B is synchronous with clock's falling edge F. The delay line of the DLL 332 controls the rising edge R and so, for the falling edge F, the PWM generator system 329 would need to rely on a perfect matching of the delay units of the DLL 332 which can be imperfect. However, to remove this error, the PWM generator system 329 uses the double frequency clock so that both Phase A and Phase B are synchronous with the rising edge R of the double frequency clock.

To perform a duty-cycle from 20% to 50% with a 2% step size, the delay line of the DLL 332 comprises 25 delay units, with the output of each respective delay unit representing a Phase nth. Eventually the phase of the output of the final delay unit will correspond to the input clock. Considering that all delays will be almost the same, a particular duty cycle is obtained with the output of the specific delay unit with simple logic in the digital core 316.

It is important to take care of the DLL 332 startup as the DLL 332 might not be able to lock a period of delay but two or more periods, taking the DLL 332 to a non-convergence zone. To avoid this issue, a start-up circuit is implemented in the PWM generator system 329 which allows the DLL 332 to start from a known and deterministic condition. The start-up circuit furthermore allows the DLL 332 to start with the minimum delay.

In examples of this disclosure, the frequency range covered by the PWM generator system 329 is extended and so the delay units in the DLL 332 can provide delays of 4 ns (for an oscillator frequency of 5 MHz) to 400 ns (for an oscillator frequency of 50 kHz). In order to accommodate for these differing delays, capacitors Cb are included in the PWM generator system 329, with the capacitor value being selected to provide the required delay.

The Phase A and Phase B are output from the DLL 332 and passed through a digital IO to the bridge IC 301 so that the Phase A and Phase B can be used to control the operation of the bridge IC 301.

The battery charging functionality of the driver 210 will now be described in more detail. The battery charging sub-system comprises the charger circuit 317 which is embedded in the PMIC 300 and controlled by a digital charge controller hosted in the PMIC 300. The charger circuit 317 is controlled by the controller 303 via the communication bus 302. The battery charging sub-system is able to charge a single cell lithium polymer (LiPo) or lithium-ion (Li-ion) battery, such as the battery 228 described above.

In this example, the battery charging sub-system is able to charge a battery or batteries with a charging current of up to 1 A from a 5V power supply (e.g. a USB power supply). One or more of the following parameters can be programmed through the communication bus 302 (I2C interface) to adapt the charge parameters for the battery:
Charge voltage can be set between 3.9V and 4.3V in 100 mV steps.
The charge current can be set between 150 mA and 1000 mA in 50 mA steps.
The pre-charge current is 1/10 of the charge current.
Pre-charge and fast charge timeouts can be set between 5 and 85 min respectively 20 and 340 min.
Optionally an external negative temperature coefficient (NTC) thermistor can be used to monitor the battery temperature.

In some examples, the battery charging sub-system reports one or more of the following events by raising an interrupt to the host controller 303:
Battery detected
Battery is being charged
Battery is fully charged
Battery is not present
Charge timeout reached
Charging supply is below the undervoltage limit

The main advantage of having the charger circuit 317 embedded in the PMIC 300, is that it allows all the programming options and event indications listed to be implemented within the PMIC 300 which guarantees the safe operation of the battery charging sub-system.

Furthermore, a significant manufacturing cost and PCB space saving can be accomplished compared with conventional mist inhaler devices which comprise discrete components of a charging system mounted separately on a PCB. The charger circuit 317 also allows for highly versatile setting of charge current and voltage, different fault timeouts and numerous event flags for detailed status analysis.

The analogue to digital converter (ADC) 318 will now be described in more detail. The inventors had to overcome significant technical challenges to integrate the ADC 318 within the PMIC 300 with the high speed oscillator 315. Moreover, integrating the ADC 318 within the PMIC 300 goes against the conventional approach in the art which relies on using one of the many discrete ADC devices that are available in the IC market.

In this example, the ADC 318 samples at least one parameter within the ultrasonic transducer driver chip (PMIC 300) at a sampling rate which is equal to the frequency of the main clock signal clk_m. In this example, the ADC 318 is a 10 bit analogue to digital converter which is able to unload digital sampling from the microprocessor 303 to save the resources of the microprocessor 303. Integrating the ADC 318 within the PMIC 300 also avoids the need to use an I2C bus that would otherwise slow down the sampling ability of the ADC (a conventional device relies on an I2C bus to communicate data between a dedicated discrete ADC and a microcontroller at a limited clock speed of typically up to 400 kHz).

In examples of this disclosure, one or more of the following parameters can be sampled sequentially by the ADC 318:
i. An rms current signal which is received at the ultrasonic transducer driver chip (PMIC 300) from an external inverter circuit which is driving an ultrasonic transducer. In this is example, this parameter is a root mean square (rms) current reported by the bridge IC 301. Sensing the rms current is important to implementing the feedback loop used for driving the ultrasound transducer 123. The ADC 318 is able to sense the rms current directly from the bridge IC 301 via a signal with minimal or no lag since the ADC 318 does not rely on this information being transmitted via an I2C bus. This provides a significant speed and accuracy benefit over conventional devices which are constrained by the comparatively low speeds of an I2C bus.
ii. The voltage of a battery connected to the PMIC 300.
iii. The voltage of a charger connected to the PMIC 300.
iv. A temperature signal, such as a temperature signal which is indicative of the PMIC 300 chip temperature. As described above, this temperature can be measured very accurately due to the temperature sensor 314 being embedded in the same IC as the oscillator 315. For example, if the PMIC 300 temperature goes up, the current, frequency and PWM are regulated by the PMIC 300 to control the transducer oscillation which in turn controls the temperature.
v. Two external pins.
vi. External NTC temperature sensor to monitor battery pack temperature.

In some examples, the ADC 318 samples one or more of the above-mentioned sources sequentially, for instance in a round robin scheme. The ADC 318 samples the sources at high speed, such as the speed of the oscillator 315 which may be up to 5 MHz or up to 105 MHz.

In some examples, the driver 210 is configured so that a user or the manufacturer of the device can specify how many samples shall be taken from each source for averaging. For instance, a user can configure the system to take 512 samples from the rms current input, 64 samples from the battery voltage, 64 from the charger input voltage, 32 samples from the external pins and 8 from the NTC pin. Furthermore, the user can also specify if one of the above-mentioned sources shall be skipped.

In some examples, for each source the user can specify two digital thresholds which divide the full range into a plurality of zones, such as 3 zones. Subsequently the user can set the system to release an interrupt when the sampled value changes zones e.g. from a zone 2 to a zone 3.

No conventional IC available in the market today can perform the above features of the PMIC 300. Sampling with such flexibility and granularity is paramount when driving a resonant circuit or component, such as an ultrasound transducer.

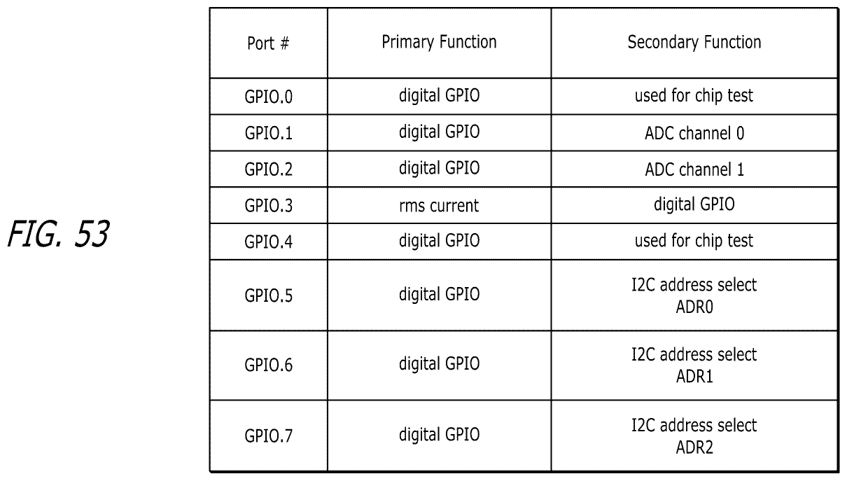

In this example, the PMIC 300 comprises an 8 bit general purpose digital input output port (GPIO). Each port can be configured as digital input and digital output. Some of the ports have an analogue input function, as shown in the table in FIG. 53.

The GPIO7-GPIO5 ports of the PMIC 300 can be used to set the device's address on the communication (I2C) bus 302. Subsequently eight identical devices can be used on the same I2C bus. This is a unique feature in the IC industry since it allows eight identical devices to be used on the same I2C bus without any conflicting addresses. This is implemented by each device reading the state of GPIO7-GPIO5 during the first 100 μs after the startup of the PMIC 300 and storing that portion of the address internally in the PMIC 300. After the PMIC 300 has been started up the GPIOs can be used for any other purpose.

As described above, the PMIC 300 comprises a six channel LED driver 320. In this example the LED driver 320 comprises N-Channel Metal-Oxide Semiconductor (NMOS) current sources which are 5V tolerant. The LED driver 320 is configured to set the LED current in four discrete levels; 5 mA, 10 mA, 15 mA and 20 mA. The LED driver 320 is configured to dim each LED channel with a 12 bit PWM signal either with or without gamma correction. The LED driver 320 is configured to vary the PWM frequency from 300 Hz to 1.5 KHz. This feature is unique in the field of ultrasonic mist inhaler devices as the functionality is embedded as a sub-system of the PMIC 300.

In this example, the PMIC 300 comprises two independent 6 Bit Digital to Analog Converters (DAC) 327, 328 which are incorporated into the PMIC 300. The purpose of the DACs 327, 328 is to output an analogue voltage to manipulate the feedback path of an external regulator (e.g. the second DC-DC converter 305 a Buck converter or a LDO). Furthermore, in some examples, the DACs 327, 328 can also be used to dynamically adjust the over current shutdown level of the bridge IC 301, as described below.

The output voltage of each DAC 327, 328 is programmable between 0V and 1.5V or between 0V and V_battery (Vbat). In this example, the control of the DAC output voltage is done via I2C commands. Having two DAC incorporated in the PMIC 300 is unique and will allow the dynamic monitoring control of the current. If either DAC 327, 328 was an external chip, the speed would fall under the same restrictions of speed limitations due to the I2C protocol. The active power monitoring arrangement of the driver 210 works with optimum efficiency if all these embedded features are in the PMIC. Had they been external components, the active power monitoring arrangement would be totally inefficient.

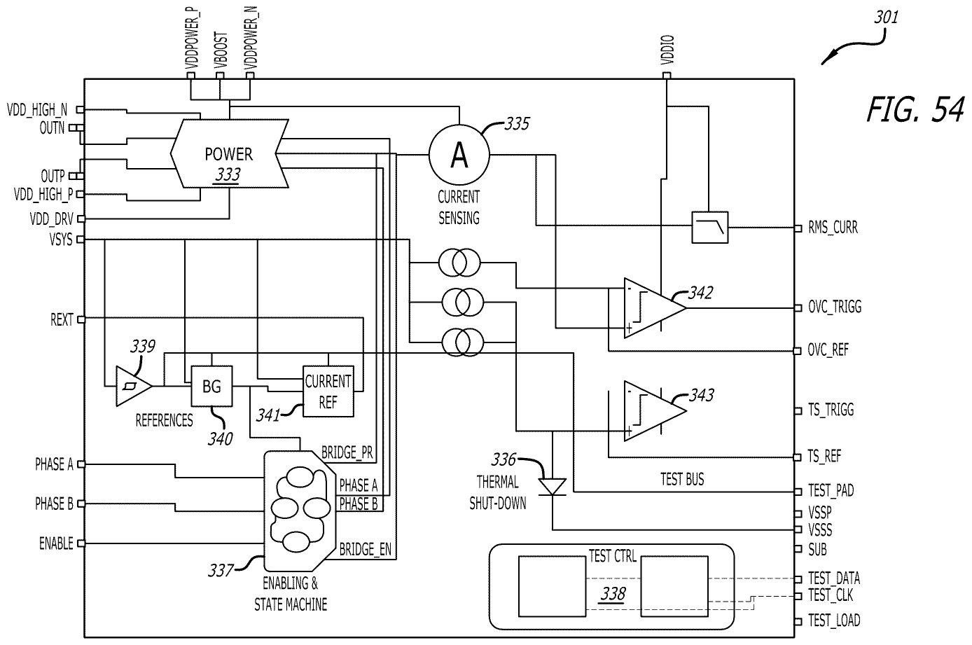

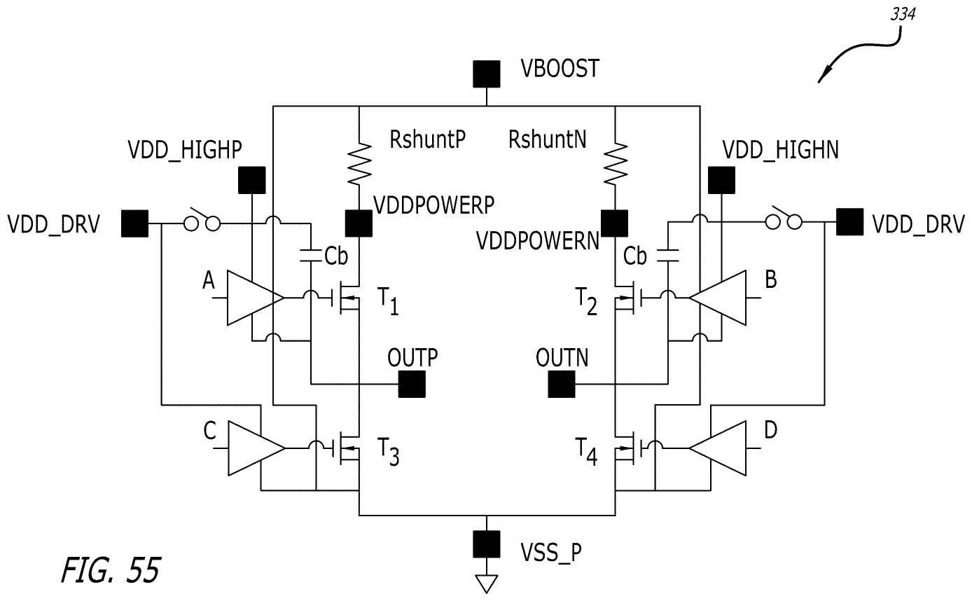

Referring now to FIG. 54 of the accompanying drawings, the bridge IC 301 is a microchip which comprises an embedded power switching circuit 333. In this example, the power switching circuit 333 is an H-bridge 334 which is shown in FIG. 55 and which is described in detail below. It is, however, to be appreciated that the bridge IC 301 of other examples may incorporate an alternative power switching circuit to the H-bridge 334, provided that the power switching circuit performs an equivalent function for generating an AC drive signal to drive the ultrasonic transducer 123.

The bridge IC 301 comprises a first phase terminal PHASE A which receives a first phase output signal Phase A from the PWM signal generator subsystem of the PMIC 300. The bridge IC 301 also comprises a second phase terminal PHASE B which receives a second phase output signal Phase B from the PWM signal generator subsystem of the PMIC 300.

The bridge IC 301 comprises a current sensing circuit 335 which senses current flow in the H-bridge 334 directly and provides an RMS current output signal via the RMS_CURR pin of the bridge IC 301. The current sensing circuit 335 is configured for over current monitoring, to detect when the current flowing in the H-bridge 334 is above a predetermined threshold. The integration of the power switching circuit 333 comprising the H-bridge 334 and the current sensing circuit 335 all within the same embedded circuit of the bridge IC 301 is a unique combination in the IC market. At present, no other integrated circuit in the IC market comprises an H-bridge with embedded circuitry for sensing the RMS current flowing through the H-bridge.

The bridge IC 301 comprises a temperature sensor 336 which includes over temperature monitoring. The temperature sensor 336 is configured to shut down the bridge IC 301 or disable at least part of the bridge IC 336 in the event that the temperature sensor 336 detects that the bridge IC 301 is operating at a temperature above a predetermined threshold. The temperature sensor 336 therefore provides an integrated safety function which prevents damage to the bridge IC 301 or other components within the driver 210 in the event that the bridge IC 301 operates at an excessively high temperature.

The bridge IC 301 comprises a digital state machine 337 which is integrally connected to the power switching circuit 333. The digital state machine 337 receives the phase A and phase B signals from the PMIC 300 and an ENABLE signal, for instance from the controller 303. The digital state machine 337 generates timing signals based on the first phase output signal Phase A and the second phase output signal Phase B.

The digital state machine 337 outputs timing signals corresponding to the phase A and phase B signals as well as a BRIDGE PR and BRIDGE EN signals to the power switching circuit 333 in order to control the power switching circuit 333. The digital state machine 337 thus outputs the timing signals to the switches T₁-T₄ of the H-bridge circuit 334 to control the switches T₁-T₄ to turn on and off in a sequence such that the H-bridge circuit outputs an AC drive signal for driving a resonant circuit, such as the ultrasonic transducer 123.

As described in more detail below, the switching sequence comprises a free-float period in which the first switch T₁ and the second switch T₂ are turned off and the third switch T₃ and the fourth switch T₄ are turned on in order to dissipate energy stored by the resonant circuit (the ultrasonic transducer 123).

The bridge IC 301 comprises a test controller 338 which enables the bridge IC 301 to be tested to determine whether the embedded components within the bridge IC 301 are operating correctly. The test controller 338 is coupled to TEST DATA, TEST CLK and TEST LOAD pins so that the bridge IC 301 can be connected to an external control device which feeds data into and out from the bridge IC 301 to test the operation of the bridge IC 301. The bridge IC 301 also comprises a TEST BUS which enables the digital communication bus within the bridge IC 301 to be tested via a TST PAD pin.

The bridge IC 301 comprises a power on reset circuit (POR) 339 which controls the startup operation of the bridge IC 301. The POR 339 ensures that the bridge IC 301 starts up properly only if the supply voltage is within a predetermined range. If the power supply voltage is outside of the predetermined range, for instance if the power supply voltage is too high, the POR 339 delays the startup of the bridge IC 301 until the supply voltage is within the predetermined range.

The bridge IC 301 comprises a reference block (BG) 340 which provides a precise reference voltage for use by the other subsystems of the bridge IC 301.

The bridge IC 301 comprises a current reference 341 which provides a precise current to the power switching circuit 333 and/or other subsystems within the bridge IC 301, such as the current sensor 335.

The temperature sensor 336 monitors the temperature of the silicon of the bridge IC 301 continuously. If the temperature exceeds the predetermined temperature threshold, the power switching circuit 333 is switched off automatically. In addition, the over temperature may be reported to an external host to inform the external host that an over temperature event has occurred.

The digital state machine (FSM) 337 generates the timing signals for the power switching circuit 333 which, in this example, are timing signals for controlling the H-bridge 334.

The bridge IC 301 comprises comparators 342,343 which compare signals from the various subsystems of the bridge IC 301 with the voltage and current references 340,341 and provide reference output signals via the pins of the bridge IC 301.

Referring again to FIG. 55 of the accompanying drawings, the H-bridge 334 of this example comprises four switches in the form of NMOS field effect transistors (FET) switches on both sides of the H-bridge 334. The H-bridge 334 comprises four switches or transistors T₁-T₄ which are connected in an H-bridge configuration, with each transistor T₁-T₄ being driven by a respective logic input A-D. The transistors T₁-T₄ are configured to be driven by a bootstrap voltage which is generated internally with two external capacitors Cb which are connected as illustrated in FIG. 55.

The H-bridge 334 comprises various power inputs and outputs which are connected to the respective pins of the bridge IC 301. The H-bridge 334 receives the programmable voltage VBOOST which is output from the second DC-DC converter 305 via a first power supply terminal, labelled VBOOST in FIG. 55. The H-bridge 334 comprises a second power supply terminal, labelled VSS_P in FIG. 55.

The H-bridge 334 comprises outputs OUTP, OUTN which are configured to connect to respective terminals of the ultrasonic transducer 123 so that the AC drive signal output from the H-bridge 334 can drive the ultrasonic transducer 123.

The switching of the four switches or transistors T₁-T₄ is controlled by switching signals from the digital state machine 337 via the logic input A-D. It is to be appreciated that, while FIG. 55 shows four transistors T₁-T₄, in other examples, the H-bridge 334 incorporates a larger number of transistors or other switching components to implement the functionality of the H-bridge.

In this example, the H-bridge 334 operates at a switching power of 22 W to 50 W in order to deliver an AC drive signal with sufficient power to drive the ultrasonic transducer 123 to generate mist optimally. The voltage which is switched by the H-bridge 334 of this example is ±15 V. In other examples, the voltage is ±20 V.

In this example, the H-bridge 334 switches at a frequency of 3 MHz to 5 MHz or up to 105 MHz. This is a high switching speed compared with conventional integrated circuit H-bridges which are available in the IC market. For instance, a conventional integrated circuit H-bridge available in the IC market today is configured to operate at a maximum frequency of only 2 MHz. Aside from the bridge IC 301 described herein, no conventional integrated circuit H-bridge available in the IC market is able to operate at a power of 22 V to 50 V at a frequency of up to 5 MHz, let alone up to 105 MHz.

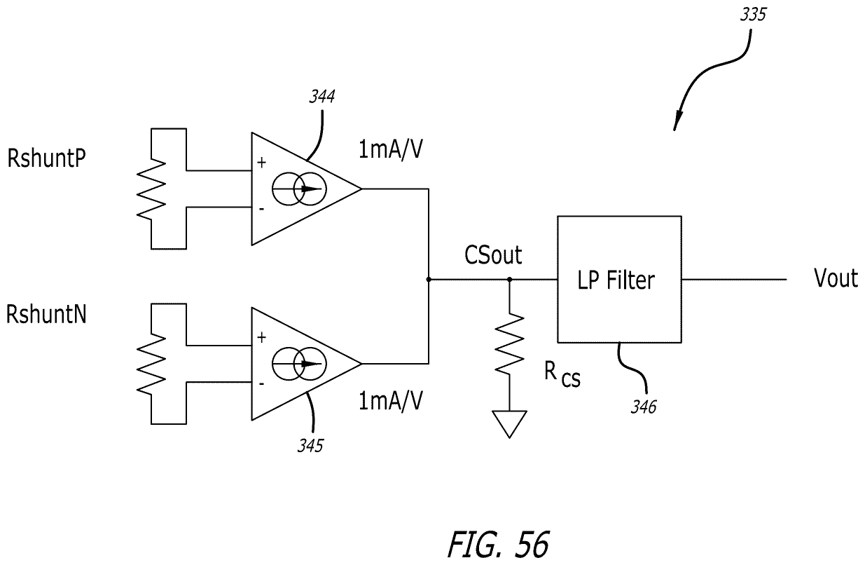

Referring now to FIG. 56 of the accompanying drawings, the current sensor 335 comprises positive and negative current sense resistors RshuntP, RshuntN which are connected in series with the respective high and low sides of the H-bridge 334, as shown in FIG. 55. The current sense resistors RshuntP, RshuntN are low value resistors which, in this example, are 0.1Ω. The current sensor 335 comprises a first voltage sensor in the form of a first operational amplifier 344 which measures the voltage drop across the first current sensor resistor RshuntP and a second voltage sensor in the form of a second operational amplifier 345 which measures the voltage drop across the second current sensor resistor RshuntN. In this example, the gain of each operational amplifier 344, 345 is 2V/V. The output of each operational amplifier 344, 345 is, in this example, 1 mA/V. The current sensor 335 comprises a pull down resistor Res which, in this example, is 2 kΩ. The outputs of the operational amplifiers 344, 345 provide an output CSout which passes through a low pass filter 346 which removes transients in the signal CSout. An output Vout of the low pass filter 346 is the output signal of the current sensor 335.

The current sensor 335 thus measures the AC current flowing through the H-bridge 334 and respectively through the ultrasonic transducer 123. The current sensor 335 translates the AC current into an equivalent RMS output voltage (Vout) relative to ground. The current sensor 335 has high bandwidth capability since the H-bridge 334 can be operated at a frequency of up to 5 MHz or, in some examples, up to 105 MHz. The output Vout of the current sensor 335 reports a positive voltage which is equivalent to the measured AC rms current flowing through the ultrasonic transducer 123. The output voltage Vout of the current sensor 335 is, in this example, fed back to the control circuitry within the bridge IC 301 to enable the bridge IC 301 to shut down the H-bridge 334 in the event that the current flowing through the H-bridge 334 and hence through the transducer 123 is in excess of a predetermined threshold. In addition, the over current threshold event is reported to the first comparator 342 in the bridge IC 301 so that the bridge IC 301 can report the over current event via the OVC TRIGG pin of the bridge IC 301.

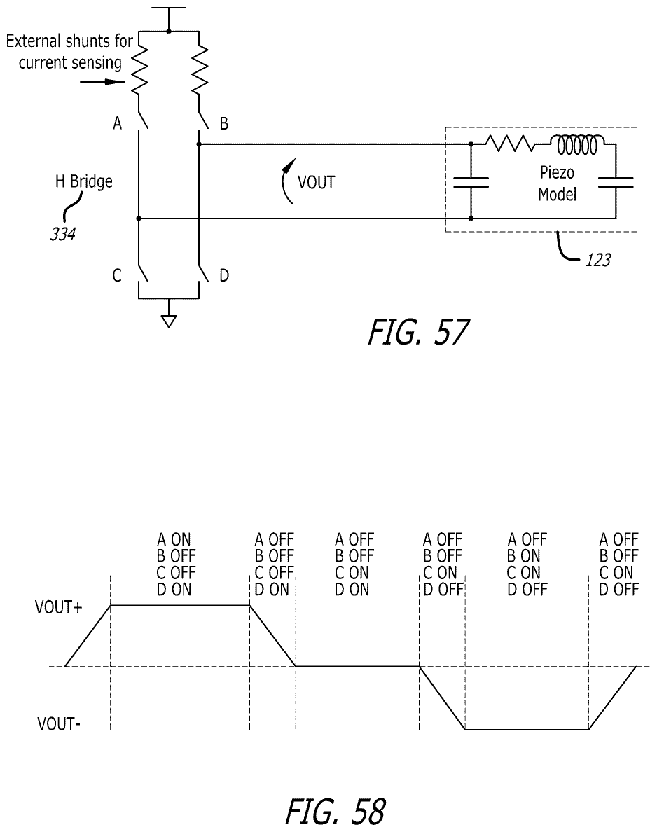

Referring now to FIG. 57 of the accompanying drawings, the control of the H-bridge 334 will now be described also with reference to the equivalent piezoelectric model of the ultrasonic transducer 123.

To develop a positive voltage across the outputs OUTP, OUTN of the H-bridge 334 as indicated by V_out in FIG. 57 (note the direction of the arrow) the switching sequence of the transistors T₁-T₄ via the inputs A-D is as follows:
1. Positive output voltage across the ultrasonic transducer 123: A-ON, B-OFF, C-OFF, D-ON
2. Transition from positive output voltage to zero: A-OFF, B-OFF, C-OFF, D-ON. During this transition, C is switched off first to minimise or avoid power loss by minimising or avoiding current flowing through A and C if there is a switching error or delay in A.
3. Zero output voltage: A-OFF, B-OFF, C-ON, D-ON. During this zero output voltage phase, the terminals of the outputs OUTP, OUTN of the H-bridge 334 are grounded by the C and D switches which remain on. This dissipates the energy stored by the capacitors in the equivalent circuit of the ultrasonic transducer, which minimises the voltage overshoot in the switching waveform voltage which is applied to the ultrasonic transducer.
4. Transition from zero to negative output voltage: A-OFF, B-OFF, C-ON, D-OFF.
5. Negative output voltage across the ultrasonic transducer 123: A-OFF, B-ON, C-ON, D-OFF

At high frequencies of up to 5 MHz or even up to 105 MHz, it will be appreciated that the time for each part of the switching sequence is very short and in the order of nanoseconds or picoseconds. For instance, at a switching frequency of 6 MHz, each part of the switching sequence occurs in approximately 80 ns.

A graph showing the output voltage OUTP, OUTN of the H-bridge 334 according to the above switching sequence is shown in FIG. 58 of the accompanying drawings. The zero output voltage portion of the switching sequence is included to accommodate for the energy stored by the ultrasonic transducer 123 (e.g. the energy stored by the capacitors in the equivalent circuit of the ultrasonic transducer). As described above, this minimises the voltage overshoot in the switching waveform voltage which is applied to the ultrasonic transducer and hence minimises unnecessary power dissipation and heating in the ultrasonic transducer.

Minimising or removing voltage overshoot also reduces the risk of damage to transistors in the bridge IC 301 by preventing the transistors from being subject to voltages in excess of their rated voltage. Furthermore, the minimisation or removal of the voltage overshoot enables the bridge IC 301 to drive the ultrasonic transducer accurately in a way which minimises disruption to the current sense feedback loop described herein. Consequently, the bridge IC 301 is able to drive the ultrasonic transducer at a high power of 22 W to 50 W or even as high as 70 W at a high frequency of up to 5 MHz or even up to 105 MHz.

The bridge IC 301 of this example is configured to be controlled by the PMIC 300 to operate in two different modes, referred to herein as a forced mode and a native frequency mode. These two modes of operation are novel over existing bridge ICs. In particular, the native frequency mode is a major innovation which offers substantial benefits in the accuracy and efficiency of driving an ultrasonic transducer as compared with conventional devices.

### Forced Frequency Mode (FFM)

In the forced frequency mode the H-bridge 334 is controlled in the sequence described above but at a user selectable frequency. As a consequence, the H-bridge transistors T₁-T₄ are controlled in a forced way irrespective of the inherent resonant frequency of the ultrasonic transducer 123 to switch the output voltage across the ultrasonic transducer 123. The forced frequency mode therefore allows the H-bridge 334 to drive the ultrasonic transducer 123, which has a resonant frequency f1, at different frequency f2.

Driving an ultrasonic transducer at a frequency which is different from its resonant frequency may be appropriate in order to adapt the operation to different applications. For example, it may be appropriate to drive an ultrasonic transducer at a frequency which is slightly off the resonance frequency (for mechanical reasons to prevent mechanical damage to the transducer). Alternatively, it may be appropriate to drive an ultrasonic transducer at a low frequency but the ultrasonic transducer has, because of its size, a different native resonance frequency.

The driver 210 controls the bridge IC 301 to drive the ultrasonic transducer 123 in the forced frequency mode in response to the configuration of the driver 210 for a particular application or a particular ultrasonic transducer. For instance, the driver 210 may be configured to operate in the forced frequency mode when the driver 210 and pod 110 is being used for a particular application, such as generating a mist from a liquid of a particular viscosity containing nicotine for delivery to a user.

### Native Frequency Mode (NFM)

The following native frequency mode of operation is a significant development and provides benefits in improved accuracy and efficiency over conventional ultrasonic drivers that are available on the IC market today.

The native frequency mode of operation follows the same switching sequence as described above but the timing of the zero output portion of the sequence is adjusted to minimise or avoid problems that can occur due to current spikes in the forced frequency mode operation. These current spikes occur when the voltage across the ultrasonic transducer 123 is switched to its opposite voltage polarity. An ultrasonic transducer which comprises a piezoelectric crystal has an electrical equivalent circuit which incorporates a parallel connected capacitor (e.g. see the piezo model in FIG. 57). If the voltage across the ultrasonic transducer is hard-switched from a positive voltage to a negative voltage, due to the high dV/dt there can be a large current flow current flow as the energy stored in the capacitor dissipates.

The native frequency mode avoids hard switching the voltage across the ultrasonic transducer 123 from a positive voltage to a negative voltage (and vice versa). Instead, prior to applying the reversed voltage, the ultrasonic transducer 123 (piezoelectric crystal) is left free-floating with zero voltage applied across its terminals for a free-float period. The PMIC 300 sets the drive frequency of the bridge IC 301 such that the bridge 334 sets the free-float period such that current flow inside the ultrasonic transducer 123 (due to the energy stored within the piezoelectric crystal) reverses the voltage across the terminals of the ultrasonic transducer 123 during the free-float period.

Consequently, when the H-bridge 334 applies the negative voltage at the terminals of the ultrasonic transducer 123 the ultrasonic transducer 123 (the capacitor in the equivalent circuit) has already been reverse charged and no current spikes occur because there is no high dV/dt.

It is, however, to be appreciated that it takes time for the charge within the ultrasonic transducer 123 (piezoelectric crystal) to build up when the ultrasonic transducer 123 is first activated. Therefore, the ideal situation in which the energy within the ultrasonic transducer 123 is to reverse the voltage during the free-float period occurs only after the oscillation inside the ultrasonic transducer 123 has built up the charge. To accommodate for this, when the bridge IC 301 activates the ultrasonic transducer 123 for the first time, the PMIC 300 controls the power delivered through the H-bridge 334 to the ultrasonic transducer 123 to a first value which is a low value (e.g. 5 V). The PMIC 300 then controls the power delivered through the H-bridge 334 to the ultrasonic transducer 123 to increase over a period of time to a second value (e.g. 15 V) which is higher than the first value in order to build up the energy stored within the ultrasonic transducer 123. Current spikes still occur during this ramp of the oscillation until the current inside the ultrasonic transducer 123 developed sufficiently. However, by using a low first voltage at start up those current spikes are kept sufficiently low to minimise the impact on the operation of the ultrasonic transducer 123.

In order to implement the native frequency mode, the driver 210 controls the frequency of the oscillator 315 and the duty cycle (ratio of turn-on time to free-float time) of the AC drive signal output from the H-bridge 334 with high precision. In this example, the driver 210 performs three control loops to regulate the oscillator frequency and the duty cycle such that the voltage reversal at the terminals of the ultrasonic transducer 123 is as precise as possible and current spikes are minimised or avoided as far as possible. The precise control of the oscillator and the duty cycle using the control loops is a significant advance in the field of IC ultrasonic drivers.

During the native frequency mode of operation, the current sensor 335 senses the current flowing through the ultrasonic transducer 123 (resonant circuit) during the free-float period. The digital state machine 337 adapts the timing signals to switch on either the first switch T₁ or the second switch T₂ when the current sensor 335 senses that the current flowing through the ultrasonic transducer 123 (resonant circuit) during the free-float period is zero.

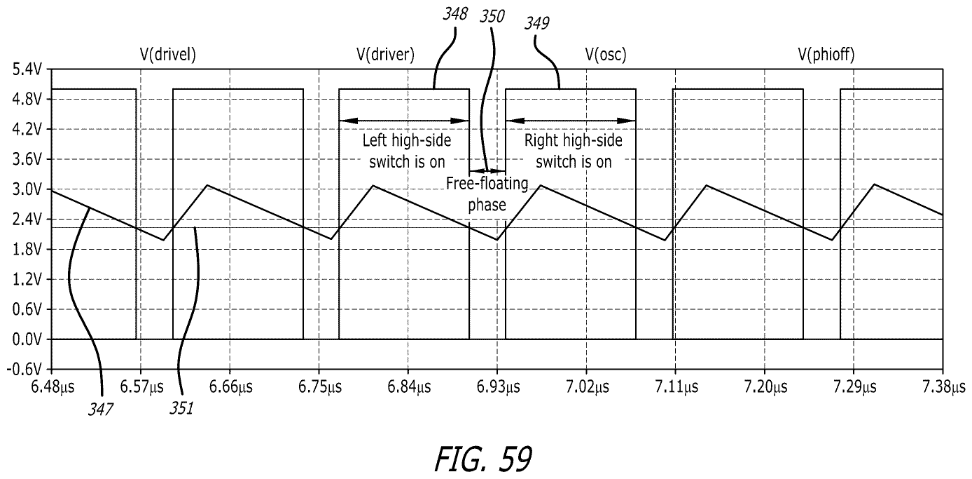

FIG. 59 of the accompanying drawings shows the oscillator voltage waveform 347 (V(osc)), a switching waveform 348 resulting from the turn-on and turn-off the left hand side high switch T₁ of the H-bridge 334 and a switching waveform 349 resulting from the turn-on and turn-off the right hand side high switch T₂ of the H-bridge 334. For an intervening free-float period 350, both high switches T₁, T₂ of the H-bridge 334 are turned off (free-floating phase). The duration of the free-float period 350 is controlled by the magnitude of the free-float control voltage 351 (Vphioff).

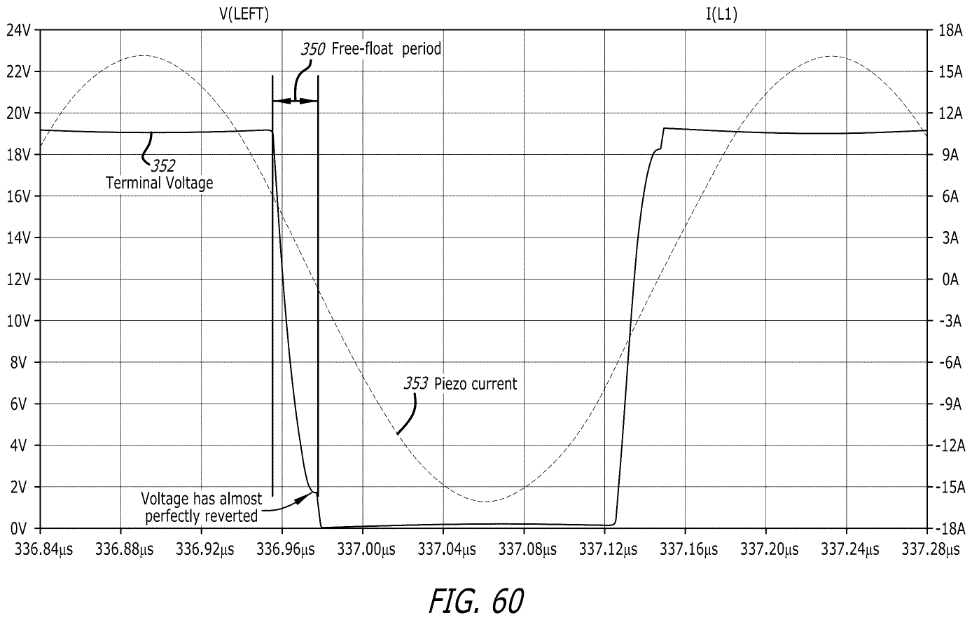

FIG. 60 of the accompanying drawings shows the voltage waveform 352 at a first terminal of the ultrasonic transducer 123 (the voltage waveform is reversed at the second terminal of the ultrasonic transducer 123) and the piezo current 353 flowing through the ultrasonic transducer 123. The piezo current 353 represents an (almost) ideal sinusoidal waveform (this is never possible in the forced frequency mode or in any bridge in the IC market).

Before the sinusoidal wave of the piezo current 353 reaches zero, the left hand side high switch T₁ of the H-bridge 334 is turned off (here, the switch T₁ is turned off when the piezo current 353 is approximately 6 A). The remaining piezo current 353 which flows within the ultrasonic transducer 123 due to the energy stored in the ultrasonic transducer 123 (the capacitor of the piezo equivalent circuit) is responsible for the voltage reversal during the free-float period 350. The piezo current 353 decays to zero during the free-float period 350 and into negative current flow domain thereafter. The terminal voltage at the ultrasonic transducer 123 drops from the supply voltage (in this case 19 V) to less than 2 V and the drop comes to a stop when the piezo current 353 reaches zero. This is the perfect time to turn on the low-side switch T₃ of the H-bridge 334 in order to minimise or avoid a current spike.

Compared to the forced frequency mode described above, the native frequency mode has at least three advantages:
1. The current spike associated with hard switching of the package capacitor is significantly reduced or avoided completely.
2. Power loss due to hard switching is almost eliminated.
3. Frequency is regulated by the control loops and will be kept close to the resonance of the piezo crystal (i.e. the native resonance frequency of the piezo crystal).

In the case of the frequency regulation by the control loops (advantage 3 above), the PMIC 300 starts by controlling the bridge IC 301 to drive the ultrasonic transducer 123 at a frequency above the resonance of the piezo crystal. The PMIC 300 then controls the bridge IC 301 to that the frequency of the AC drive signal decays/reduces during start up. As soon as the frequency approaches resonance frequency of the piezo crystal, the piezo current will develop/increase rapidly. Once the piezo current is high enough to cause the desired voltage reversal, the frequency decay/reduction is stopped by the PMIC 300. The control loops of the PMIC 300 then take over the regulation of frequency and duty cycle of the AC drive signal.

In the forced frequency mode, the power delivered to the ultrasonic transducer 123 is controlled through the duty cycle and/or a frequency shift and/or by varying the supply voltage. However, in this example in the native frequency mode the power delivered to the ultrasonic transducer 123 controlled only through the supply voltage.

In this example, during a setup phase of operation of the driver 210, the bridge IC 301 is configured to measure the length of time taken for the current flowing through the ultrasonic transducer 123 (resonant circuit) to fall to zero when the first switch T₁ and the second switch T₂ are turned off and the third switch T₃ and the fourth switch T₄ are turned on. The bridge IC 301 then sets the length of time of the free-float period to be equal to the measured length of time.

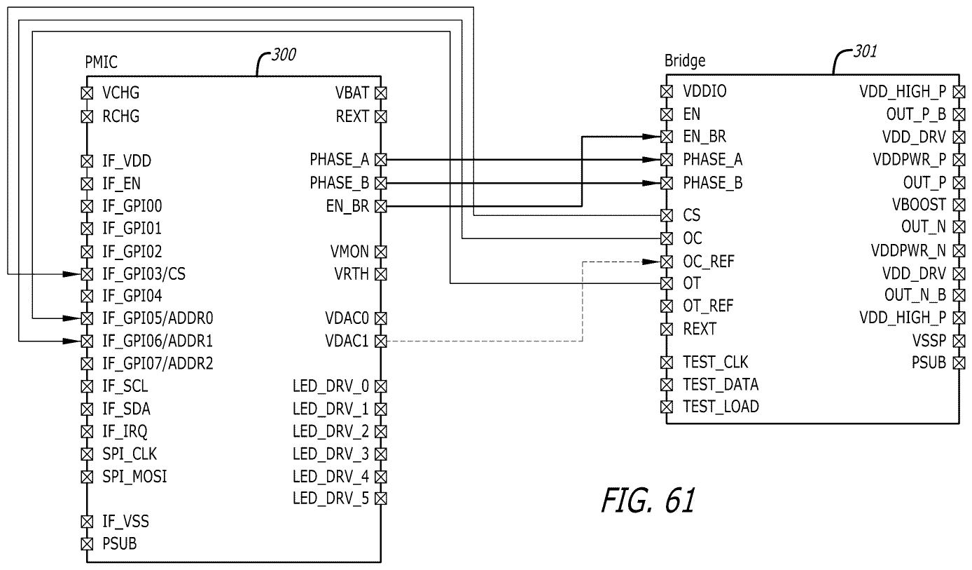

Referring now to FIG. 61 of the accompanying drawings, the PMIC 300 and the bridge IC 301 of this example are designed to work together as a companion chip set. The PMIC 300 and the bridge IC 301 are connected together electrically for communication with one another. In this example, there are interconnections between the PMIC 300 and the bridge IC 301 which enable the following two categories of communication:
1. control signals
2. feedback signals

The connections between the PHASE_A and PHASE_B pins of the PMIC 300 and the bridge IC 301 carry the PWM modulated control signals which drive the H-bridge 334. The connection between the EN_BR pins of the PMIC 300 and the bridge IC 301 carries the EN_BR control signal which triggers the start of the H-bridge 334. The timing between the PHASE_A, PHASE_B and EN_BR control signals is important and handled by the digital bridge control of the PMIC 300.

The connections between the CS, OC and OT pins of the PMIC 300 and the bridge IC 301 carry CS (current sense), OC (over current) and OT (over temperature) feedback signals from the bridge IC 301 back to the PMIC 300. Most notably, the CS (current sense) feedback signal comprises a voltage equivalent to the rms current flowing through the ultrasonic transducer 123 which is measured by the current sensor 335 of the bridge IC 301.

The OC (over current) and OT (over temperature) feedback signals are digital signals indicating that either an over current or an over voltage event has been detected by the bridge IC 301. In this example, the thresholds for the over current and over temperature are set with an external resistor. Alternatively, the thresholds can also be dynamically set in response to signals passed to the OC_REF pin of the bridge IC 301 from one of the two DAC channels VDAC0, VDAC1 from the PMIC 300.

In this example, the design of the PMIC 300 and the bridge IC 301 allow the pins of these two integrated circuits to be connected directly to one another (e.g. via copper tracks on a PCB) so that there is minimal or no lag in the communication of signals between the PMIC 300 and the bridge IC 301. This provides a significant speed advantage over conventional bridges in the IC market which are typically controlled by signals via a digital communications bus. For example, a standard I2C bus is clocked at only 400 kHz, which is too slow for communicating data sampled at the high clock speeds of up to 5 MHz of examples of this disclosure.

While examples of this disclosure have been described above in relation to the microchip hardware, it is to be appreciated that other examples of this disclosure comprise a method of operating the components and subsystems of each microchip to perform the functions described herein. For instance, the methods of operating the PMIC 300 and the bridge IC 301 in either the forced frequency mode or the native frequency mode.

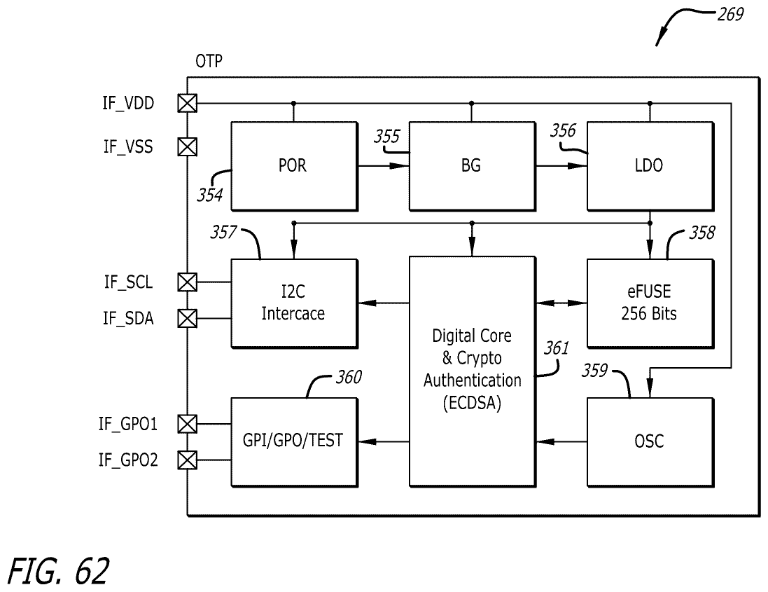

Referring now to FIG. 62 of the accompanying drawings, the OTP IC 269 comprises a power on reset circuit (POR) 354, a bandgap reference (BG) 355, a cap-less low dropout regulator (LDO) 356, a communication (e.g. I2C) interface 357, a one-time programmable memory bank (eFuse) 358, an oscillator 359 and a general purpose input-output interface 360. The OTP IC 269 also comprises a digital core 361 which includes a cryptographic authenticator. In this example, the cryptographic authenticator uses the Elliptic Curve Digital Signature Algorithm (ECDSA) for encrypting/decrypting data stored within the OTP IC 269 as well as data transmitted to and from the OTP IC 269.

The POR 354 ensures that the OTP IC 269 starts up properly only if the supply voltage is within a predetermined range. If the supply voltage is outside the predetermined range, the POR 354 resets the OTP IC 269 and waits until the supply voltage is within the predetermined range.

The BG 355 provides precise reference voltages and currents to the LDO 356 and to the oscillator 359. The LDO 356 supplies the digital core 361, the communication interface 357 and the eFuse memory bank 358.

The OTP IC 269 is configured to operate in at least the following modes:
Fuse Programming (Fusing): During efuse programming (programming of the one time programmable memory) a high current is required to burn the relevant fuses within the eFuse memory bank 358. In this mode higher bias currents are provided to maintain gain and bandwidth of the regulation loop.
Fuse Reading: In this mode a medium level current is required to maintain efuse reading within the eFuse memory bank 358. This mode is executed during the startup of the OTP IC 269 to transfer the content of the fuses to shadow registers. In this mode the gain and bandwidth of the regulation loop is set to a lower value than in the Fusing Mode.
Normal Operation: In this mode the LDO 356 is driven in a very low bias current condition to operate the OTP IC 269 with low power so that the OTP IC 269 consumes as little power as possible.

The oscillator 359 provides the required clock for the digital core/engine 361 during testing (SCAN Test), during fusing and during normal operation. The oscillator 359 is trimmed to cope with the strict timing requirements during the fusing mode.

In this example, the communication interface 357 is compliant with the FM+ specification of the I2C standard but it also complies with slow and fast mode. The OTP IC 269 uses the communication interface 357 to communicate with the driver 210 (the Host) for data and key exchange.

The digital core 361 implements the control and communication functionality of the OTP IC 269. The cryptographic authenticator of the digital core 361 enables the OTP IC 269 to authenticate itself (e.g. using ECDSA encrypted messages) with the driver 210 (e.g. for a particular application) to ensure that the OTP IC 269 is genuine and that the OTP IC 269 is authorised to connect to the driver 210 (or another product).

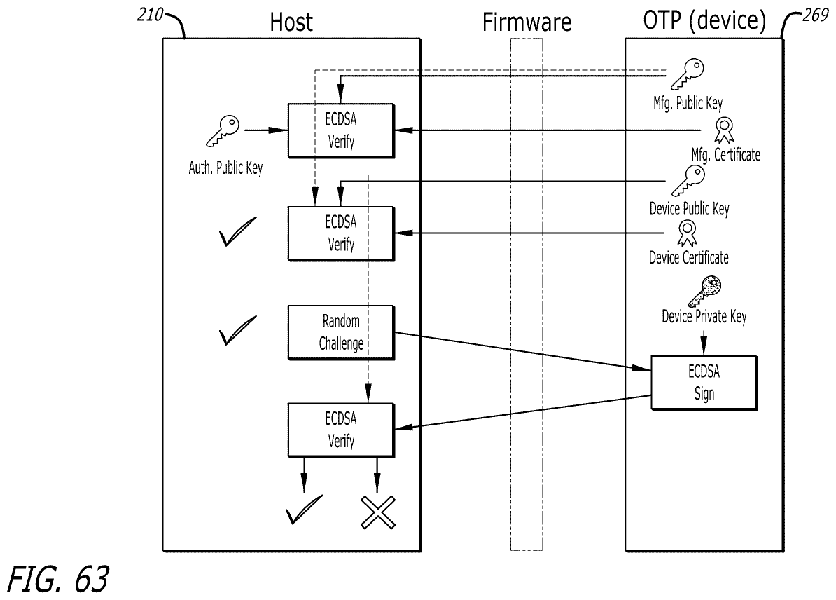

With reference to FIG. 63 of the accompanying drawings, the OTP IC 269 performs the following PKI procedure in order to authenticate the OTP IC 269 for use with a Host (e.g. the driver 210):
1. Verify Signer Public Key: The Host requests the Manufacturing Public key and Certificate. The Host verifies the certificate with the Authority Public key.
2. Verify Device Public Key: If the verification is successful, the Host requests the Device Public key and Certificate. The Host verifies the certificate with the Manufacturing Public key.
3. Challenge-Response: If the verification is successful, the Host creates a random number challenge and sends it to the Device. The End Product signs the random number challenge with the Device Private key.
4. The signature is sent back to the Host for verification using the Device Public key.

If all steps of the authentication procedure complete successfully then the Chain of Trust has been verified back to the Root of Trust and the OTP IC 269 is successfully authenticated for use with the Host. However, if any of the steps of the authentication procedure fail then the OTP IC 269 is not authenticated for use with the Host and use of the device incorporating the OTP IC 269 is restricted or prevented.

The driver 210 comprises an AC driver for converting a voltage from the battery into an AC drive signal at a predetermined frequency to drive the ultrasonic transducer.

The driver 210 comprises an active power monitoring arrangement for monitoring the active power used by the ultrasonic transducer (as described above) when the ultrasonic transducer is driven by the AC drive signal. The active power monitoring arrangement provides a monitoring signal which is indicative of an active power used by the ultrasonic transducer.

The processor within the driver 210 controls the AC driver and receives the monitoring signal drive from the active power monitoring arrangement.

The memory of driver 210 stores instructions which, when executed by the processor, cause the processor to:
A. control the AC driver to output an AC drive signal to the ultrasonic transducer at a predetermined sweep frequency;
B. calculate the active power being used by the ultrasonic transducer based on the monitoring signal;
C. control the AC driver to modulate the AC drive signal to maximise the active power being used by the ultrasonic transducer;
D. store a record in the memory of the maximum active power used by the ultrasonic transducer and the sweep frequency of the AC drive signal;
E. repeat steps A-D for a predetermined number of iterations with the sweep frequency incrementing or decrementing with each iteration such that, after the predetermined number of iterations has occurred, the sweep frequency has been incremented or decremented from a start sweep frequency to an end sweep frequency;
F. identify from the records stored in the memory the optimum frequency for the AC drive signal which is the sweep frequency of the AC drive signal at which a maximum active power is used by the ultrasonic transducer; and
G. control the AC driver to output an AC drive signal to the ultrasonic transducer at the optimum frequency to drive the ultrasonic transducer to atomise a liquid.

In some examples, the active power monitoring arrangement comprises a current sensing arrangement for sensing a drive current of the AC drive signal driving the ultrasonic transducer, wherein the active power monitoring arrangement provides a monitoring signal which is indicative of the sensed drive current.

In some examples, the current sensing arrangement comprises an Analog-to-Digital Converter which converts the sensed drive current into a digital signal for processing by the processor.

In some examples, the memory stores instructions which, when executed by the processor, cause the processor to: repeat steps A-D above with the sweep frequency being incremented from a start sweep frequency of 2900 kHz to an end sweep frequency of 2960 KHz.

In some examples, the memory stores instructions which, when executed by the processor, cause the processor to: repeat steps A-D above with the sweep frequency being incremented from a start sweep frequency of 2900 kHz to an end sweep frequency of 3100 kHz.

In some examples, the memory stores instructions which, when executed by the processor, cause the processor to: in step G, control the AC driver to output an AC drive signal to the ultrasonic transducer at frequency which is shifted by a predetermined shift amount from the optimum frequency.

In some examples, the predetermined shift amount is between 1-10% of the optimum frequency.

### 2. Control and Information (CI) Section

The Control and Information section comprises an external EEPROM for data storage, LEDs for user indications, an air flow sensor for airflow detection and a Bluetooth Low Energy (BLE) capable microcontroller for constant monitoring and managing of the aerosolisation section.

The air flow sensor used in the device serves two purposes. The first purpose is to prevent unwanted and accidental start of the sonic engine (driving the ultrasonic transducer). This functionality is implemented in the processing arrangement of the device, but optimised for low power, to constantly measures environmental parameters such as temperature and ambient pressure with internal compensation and reference setting in order to accurately detect and categorise what is called a true inhalation.

Unlike all the other e-smoking devices on the market, this solution uses the strength of a micro-controller to allow the use of only one sensor.

The second purpose of the air flow sensor is to be able to monitor not only the exact duration of the inhalations by the user for precise inhalation volume measurement, but also to be able to determine the strength of the user inhalation which is a critical information if the device is being used as part of a smoking cessation program. All in all, we are able to completely draw the pressure profile of every inhalation and anticipate the end of an inhalation for both aerosolisation optimisation and nicotine dependency comprehension.

This was possible with the usage of a Bluetooth™ Low Energy (BLE) microcontroller. Indeed, this enables the setting to provide extremely accurate inhalation times, optimised aerosolisation, monitor numerous parameters to guarantee safe misting and prevent the use of non-genuine e-liquids or aerosol chambers and protect both the device against over-heating risks and the user against over-misting in one shot unlike any other products on the market.

The use of the BLE microcontroller allows over-the-air update to continuously provide improved software to users based on anonymised data collection and trained AI for PZT modelling.

### 3. Power Management (PM) Section

The Power Management section is constituted by the 3.7V LiPo battery path to a low dropout regulator (LDO) that powers the Control and Information section and a battery management system (BMS) that provides high level of protection and charging to the internal LiPo battery.

The components in this section have been selected carefully and thoroughly to be able to provide such an integrated and compact device while providing high power to the sonication section and ensuring a steady powering of the control and information section.

Indeed, when providing high power to the aerosolisation section from a 3.7V LiPo battery, the supply voltage varies a lot during operation. Without a low dropout regulator, the Control and Information section could not be powered with a mandatory steady supply when the battery voltage drops to as low as 0.3V above the minimum ratings of the components in this section, which is why the LDO plays a crucial role here. A loss in the CI section would disturb or even stop the functioning of the entire device.

This is why the careful selection of components not only ensures high reliability of the device but also allows it to work under harsh conditions and for a longer consecutive time between recharge.

### Controlled Aerosolisation

The device is a precise, reliable and a safe aerosolisation solution for smoke cessation programs and daily customer usage and, as such, must provide a controlled and trusted aerosolisation.

This is performed through an internal method that can be broken apart into several sections as follows:

### 1. Sonication

In order to provide the most optimal aerosolisation the ultrasonic transducer (PZT) needs to vibrate in the most efficient way.

### Frequency

The electromechanical properties of piezoelectrical ceramics state that the component has the most efficiency at the resonant frequency. But also, vibrating a PZT at resonance for a long duration will inevitably end with the failure and breaking of the component which renders the aerosol chamber unusable.

Another important point to consider when using piezoelectrical materials is the inherent variability during manufacturing and its variability over temperature and lifetime.

Resonating a PZT at 3 MHz in order to create droplets of a size <1 μm requires an adaptive method in order to locate and target the ‘sweet spot’ of the particular PZT inside every aerosol chamber used with the device for every single inhalation.

### Sweep

Because the device has to locate the ‘sweet spot’ for every single inhalation and because of over-usage, the PZT temperature varies as the device uses an in-house double sweep method.

The first sweep is used when the device has not been used with a particular aerosol chamber for a time that is considered enough for all the thermal dissipation to occur and for the PZT to cool down to ‘default temperature’. This procedure is also called a cold start. During this procedure the PZT needs a boost in order to produce the required aerosol. This is achieved by only going over a small subset of Frequencies between 2900 kHz to 2960 kHz which, considering extensive studies and experiments, covers the resonant point.

For each frequency in this range, the sonic engine in activated and the current going through the PZT is actively monitored and stored by the controller via an Analog-to-Digital Converter (ADC), and converted back to current in order to be able to precisely deduct the Power used by the PZT.

This yields the cold profile of this PZT regarding frequency and the Frequency used throughout the inhalation is the one that uses the most current, meaning the lowest impedance Frequency.

The second sweep is performed during any subsequent inhalation and cover the entire range of frequencies between 2900 kHz to 3100 kHz due to the modification of the PZT profile with regards to temperature and deformation. This hot profile is used to determine the shift to apply.

### Shift

Because the aerosolisation must be optimal, the shift is not used during any cold inhalation and the PZT will hence vibrate at resonant frequency. This can only happen for a short and unrepeated duration of time otherwise the PZT would inevitably break.

The shift however is used during most of inhalations as a way to still target a low impedance frequency, thus resulting in quasi-optimal operation of the PZT while protecting it against failures.

Because the hot and cold profiles are stored during inhalation the controller can then select the proper shifted frequency according to the measured values of current through the PZT during sweep and ensure a safe mechanical operation.

The selection of the direction to shift is crucial as the piezoelectrical component behaves in a different way if outside the duplet resonant/anti-resonant frequency or inside this range. The selected shift should always be in this range defined by Resonant to anti-Resonant frequencies as the PZT is inductive and not capacitive.

Finally, the percentage to shift is maintained below 10% in order to still remain close to the lowest impedance but far enough of the resonance.

### Adjustment

Because of the intrinsic nature of PZTs, every inhalation is different. Numerous parameters other than the piezoelectrical element influence the outcome of the inhalation, like the amount of e-liquid remaining inside the aerosol chamber, the wicking state of the gauze or the battery level of the device.

As of this, the device permanently monitors the current used by the PZT inside the aerosol chamber and the controller constantly adjusts the parameters such as the frequency and the Duty Cycle in order to provide the aerosol chamber with the most stable power possible within a pre-defined range that follows the studies and experimental results for most optimal safe aerosolisation.

### Battery Monitoring

In order to provide an AC voltage of 15V and maintain a current inside the PZT around 2.5 A, the current drawn from the battery reaches around 7 to 8 Amps, which in turn, creates a drop in the battery voltage. Any common LiPo battery would not sustain this demanding resource for the duration of an inhalation that can top 6s.

This is the reason why a custom LiPo battery is developed that can handle around 11 Amps, which is 50% more than the maximum allowed in the PZT at all time, while still being simple to use in compact and integrated portable device.

Because the battery voltage drops and varies a lot when activating the sonication section, the controller constantly monitors the power used by the PZT inside the aerosol chamber to ensure a proper but also safe aerosolisation.

And because the key to aerosolisation is control, the device ensures first that the Control and Information section of the device always function and does not stop in the detriment of the sonication section.

This is why the adjustment method also takes into great account the real time battery level and, if need be, modifies the parameters like the Duty Cycle to maintain the battery at a safe level, and in the case of a low battery before starting the sonic engine, the Control and Information section will prevent the activation.

### Power Control

As being said, the key to aerosolisation is control and the method used in the device is a real time multi-dimensional function that takes into account the profile of the PZT, the current inside the PZT and the battery level of the device at all time.

All this is only achievable thanks to the use of a controller that can monitor and control every element of the device to produce an optimal inhalation.

### 1. Inhalation Control

The device is a safe device and confirmed by BNS (Broughton Nicotine Services) report, but in order to guarantee the safety of misting and the integrity of both the aerosol chamber and the device, each inhalation has to be controlled.

### Inhalation Duration

In order to reduce the exposure to carbonyls and other toxic components that might result from the heating of e-liquid, the maximum duration of an inhalation is set to 6 seconds which completely ensure that the exposure to these components is contained.

### Interval

Because the device relies on a piezoelectrical component, the device prevents the activation of the sonication section if an inhalation stops. The safety delay in between two inhalations is adaptive depending on the duration of the previous one. This allows the gauze to wick properly before the next activation.

With this functioning, the device can safely operate and the aerosolisation is rendered more optimal with no risk of breaking the PZT element nor exposing the user to toxic components.

### Connectivity (BLE)

The device Control and Information section is composed of a wireless communication system in the form of a Bluetooth Low Energy capable controller. The wireless communication system is in communication with the processor of the device and is configured to transmit and receive data between the driver 210 and a computing device, such as a smartphone.

The connectivity via Bluetooth Low Energy to a companion mobile application ensures that only small power for this communication is required thus allowing the device to remain functioning for a longer period of time if not used at all, compared to traditional wireless connectivity solutions like Wi-Fi, classic Bluetooth, GSM or even LTE-M and NB-IOT.

Most importantly, this connectivity is what enables the OTP as a feature and the complete control and safety of the inhalations. Every data from resonant frequency of an inhalation to the one used, or the negative pressure created by the user and the duration are stored and transferred over BLE for further analysis and improvements of the embedded software.

Moreover, all these information are crucial when the device is used in smoke cessation programs because it gives doctors and users all the information regarding the process of inhalation and the ability to track in real-time the prescriptions and the usage.

Finally, this connectivity enables the update of the embedded firmware inside the device and over the air (OTA), which guarantees that the latest versions can always be deployed rapidly. This gives great scalability to the device and insurance that the device is intended to be maintained.

### Data Collection for Clinical Smoke Cessation Purposes

The device can collect user data such as number of puffs and puff duration in order to determine the total amount of nicotine consumed by the user in a session.

This data can be interpreted by an algorithm that sets consumption limits per time period based on a physician's recommendations.

This will allow a controlled dose of nicotine to be administered to the user that is controlled by a physician or pharmacist and cannot be abused by the end user.

The physician would be able to gradually lower dosages over time in a controlled method that is both safe for the user and effective in providing therapeutic smoke cessation doses.

### Puff Limitations

The process of ultrasonic cavitation has a significant impact on the nicotine concentration in the produced mist.

A device limitation of <7 second puff durations will limit the user to exposure of carbonyls commonly produced by electronic nicotine delivery systems.

Based on Broughton Nicotine Services' experimental results, after a user performs 10 consecutive puffs of <7 seconds, the total amount of carbonyls is <2.67 μg/10 puffs (average: 1.43 μg/10 puffs) for formaldehyde, <0.87 μg/10 puffs (average: 0.50 μg/10 puffs) for acetaldehyde, <0.40 μg/10 puffs (average: 0.28 μg/10 puffs) for propionaldehyde, <0.16 μg/10 puffs (average: 0.16 μg/10 puffs) for crotonaldehyde, <0.19 μg/10 puffs (average: 0.17 μg/10 puffs) for butyraldehyde, <0.42 μg/10 puffs (average: 0.25 μg/10 puffs) for diacetyl, and acetylpropionyl was not detected at all in the emissions after 10 consecutive <7 second puffs.

Because the aerosolisation of the e-liquid is achieved via the mechanical action of the piezoelectric disc and not due to the direct heating of the liquid, the individual components of the e-liquid (propylene glycol, vegetable glycerine, flavouring components, etc.) remain largely in-tact and are not broken into smaller, harmful components such as acrolein, acetaldehyde, formaldehyde, etc. at the high rate seen in traditional ENDS.

In order to limit the user's exposure to carbonyls while using the ultrasonic device, puff length is limited to 6 seconds maximum so that the above results would be the absolute worst-case scenario in terms of exposure.

A device of other examples of this disclosure comprises most of or preferably all of the elements of the driver 210 described above, but with the memory of the driver 210 storing instructions which, when executed by the processor, provide additional functionality to the driver.

In one example, the driver 210 comprises an active power monitor which incorporates a current sensor, such as the current sensor 335 described above, for sensing an rms drive current of the AC drive signal driving the ultrasonic transducer 123. The active power monitor provides a monitoring signal which is indicative of the sensed drive current, as described above.

The additional functionality of this example enables the driver to monitor the operation of the ultrasonic transducer while the ultrasonic transducer is activated. The driver 210 calculates an effectiveness value or quality index which is indicative of how effective the ultrasonic transducer is operating to atomise a liquid within the device. The device uses the effectiveness value to calculate the actual amount of mist that was generated over the duration of activation of the ultrasonic transducer.

Once the actual amount of mist has been calculated, the device is configured to calculate the actual amount of nicotine which was present in the mist and hence the actual amount of nicotine which was inhaled by a user based on the concentration of the nicotine in the liquid. Knowing the exact amount of nicotine which is delivered to a user is particularly important when the driver and pod is being used as part of a smoke cessation program which gradually limits the amount of nicotine which is delivered to a user over a period of time. Knowing the exact amount of nicotine which is delivered to a user during each inhalation or puff allows for the smoke cessation program to operate more accurately and effectively compared with using a conventional device which simply counts the number of inhalations or puffs, with each inhalation or puff assumed to deliver the same quantity of nicotine to a user.

In practice, as described above, there are many different factors which affect the operation of an ultrasonic transducer and which have an impact on the amount of mist which is generated by the ultrasonic transducer and hence the actual amount of nicotine which is delivered to a user.

For instance, if an ultrasonic transducer within a pod is not operating in an optimal manner due to a low charge in the battery reducing the current flowing through the ultrasonic transducer, a lower amount of mist will be generated and a lower amount of nicotine will be delivered to a user compared with if the device was operating optimally. If the driver and pod is being used in a smoke cessation program, the device may thus allow a greater number of puffs for a user in order to deliver a set amount of nicotine to the user over a period of time compared with the number of puffs that would be permitted if the ultrasonic transducer was operating optimally. This enables the smoke cessation program to operate more effectively and precisely compared with a conventional program which relies on using a device which simply counts and restricts the number of puffs taken by a user.

The configuration of the driver and a method of generating mist using the mist inhaler device of some examples will now be described in detail below.

In some examples, the memory of the driver 210 further stores instructions which, when executed by the processor, cause the processor to activate the pod 110 for a first predetermined length of time.

The executed instructions cause the processor to sense, using a current sensor, periodically during the first predetermined length of time the current of the AC drive signal flowing through the ultrasonic transducer 123 and storing periodically measured current values in the memory.

In some examples, the executed instructions cause the processor to calculate an effectiveness value using the current values stored in the memory. The effectiveness value is indicative of the effectiveness of the operation of the ultrasonic transducer at atomising the liquid.

In some examples, the executed instructions cause the processor to calculate the effectiveness value using this equation:

$${Q}_{I} = \frac{{\sum }_{t = 0}^{t = D} \frac{\sqrt{{Q}_{A ( t )}^{2} + {Q}_{F ( t )}^{2}}}{\sqrt{2}}}{N}$$

where:
Q_{I} is the effectiveness value,
Q_{F} is a frequency sub-effectiveness value which is based on the monitored frequency value (the frequency at which the ultrasonic transducer 123 is being driven),
Q_{A} is an analogue to digital converter sub-effectiveness value which is based on the measured current value (the rms current flowing through the ultrasonic transducer 123),
t=0 is the start of the first predetermined length of time,
t=D is the end of the first predetermined length of time,
N is the number of periodic measurements (samples) during the first predetermined length of time, and
\sqrt{2} is a normalization factor.

In some examples, the memory stores instructions which, when executed by the processor, cause the processor to measure periodically during the first predetermined length of time the duty cycle of the AC drive signal driving the ultrasonic transducer and storing periodically measured duty cycle values in the memory. The driver then modifies the analogue to digital converter sub-effectiveness value Q_{A} based on the current values stored in the memory. Consequently, the driver of this example takes into account variations in the duty cycle which may occur throughout the activation of the ultrasonic transducer 123 when the device calculates the effectiveness value. The driver can therefore calculate the actual amount of mist which is generated accurately by taking into account variations in the duty cycle of the AC drive signal which may occur while the ultrasonic transducer is activated.

In one example, the memory stores instructions which, when executed by the processor, cause the processor to measure periodically during the first predetermined length of time a voltage of a battery which is powering the driver and storing periodically measured battery voltage values in the memory. The driver then modifies the analogue to digital converter sub-effectiveness value Q_{A} based on the battery voltage values stored in the memory. Consequently, the driver of this example takes into account variations in the battery voltage which may occur throughout the activation of the ultrasonic transducer 123 when the device calculates the effectiveness value. The driver can therefore calculate the actual amount of mist which is generated accurately by taking into account variations in the battery voltage which may occur while the ultrasonic transducer is activated.

The effectiveness value is used by the driver as a weighting to calculate the actual amount of mist generated by the driver by proportionally reducing a value of a maximum amount of mist that would be generated if the device was operating optimally.

In one example, the memory stores instructions which, when executed by the processor, cause the processor to measure periodically during the first predetermined length of time the frequency of the AC drive signal driving the ultrasonic transducer 123 and storing periodically measured frequency values in the memory. The device then calculates the effectiveness value using the using the frequency values stored in the memory, in addition to the current values as described above.

In one example, the memory stores instructions which, when executed by the processor, cause the processor to calculate a maximum mist amount value that would be generated if the ultrasonic transducer 123 was operating optimally over the duration of the first predetermined length of time. In one example, the maximum mist amount value is calculate based on modelling which determines the maximum amount of mist which would be generated when the ultrasonic transducer was operating optimally.

Once the maximum mist amount value has been calculated, the driver can calculate an actual mist amount value by reducing the maximum mist amount value proportionally based on the effectiveness value to determine the actual mist amount that was generated over the duration of the first predetermined length of time.

Once the actual mist amount has been calculated, the driver can calculate a nicotine amount value which is indicative of the amount of nicotine in the actual mist amount that was generated over the duration of the first predetermined length of time. The driver then stores a record of the nicotine amount value in the memory. In this way, the driver can record accurately the actual amount of nicotine which has been delivered to a user in each inhalation or puff.

In one example, the memory stores instructions which, when executed by the processor, cause the processor to selecting a second predetermined length of time in response to the effectiveness value. In this case, the second predetermined length of time is a length of time over which the ultrasonic transducer 123 is activated during a second inhalation or puff by a user. In one example, the second predetermined length of time is equal to the first predetermined length of time but with the time reduced or increased proportionally according to the effectiveness value. For instance, if the effectiveness value indicates that the ultrasonic transducer 123 is not operating effectively, the second predetermined length of time is made longer by the effectiveness value such that a desired amount of mist is generated during the second predetermined length of time.

When it comes to the next inhalation, the driver activates the pod for the second predetermined length of time so that the pod generates a predetermined amount of mist during the second predetermined length of time. The driver thus controls the amount of mist generated during the second predetermined length of time accurately, taking into account the various parameters which are reflected by the effectiveness value which affect the operation of the driver.

In one example, the memory stores instructions which, when executed by the processor, cause the processor to activate the pod for a plurality of predetermined lengths of time. For instance, the pod is activated during a plurality of successive inhalations or puffs by a user.

The driver stores a plurality of nicotine amount values in the memory, each nicotine amount value being indicative of the amount of nicotine in the mist that was generated over the duration of a respective one of the predetermined lengths of time. In one example, the driver prevents further activation of the pod for a predetermined duration if the total amount of the nicotine in the mist that was generated over the duration of the predetermined lengths of time is equal to or greater than a predetermined threshold. In one example, the predetermined duration is a duration in the range of 1 to 24 hours. In other examples, the predetermined duration is 24 hours or 12 hours.

The mist inhaler of some examples of this disclosure is configured to transmit data indicative of the nicotine amount values from the pod to a computing device (e.g. via Bluetooth™ Low Energy communication) for storage in a memory of the computing device (e.g. a smartphone). An executable application running on the computing device can thus log the amount of nicotine which has been delivered to a user. The executable application can also control the operation of the driver to limit the activation of the driver to restrict the amount of nicotine being delivered to a user over a period of time, for instance as part of a smoking cessation program.

The driver of some examples of this disclosure is therefore configured to prevent further activation once a user has consumed a set amount of nicotine during a set timeframe, such as the amount of nicotine consumed during a day.

All of the above applications involving ultrasonic technology can benefit from the optimisation achieved by the frequency controller which optimises the frequency of sonication for optimal performance.

The driver 210 and pod 110 of some examples has particular applicability for those who use electronic inhalers as a means to quit smoking and reduce their nicotine dependency. The ultrasonic mist inhaler 100 provides a way to gradually taper the dose of nicotine.

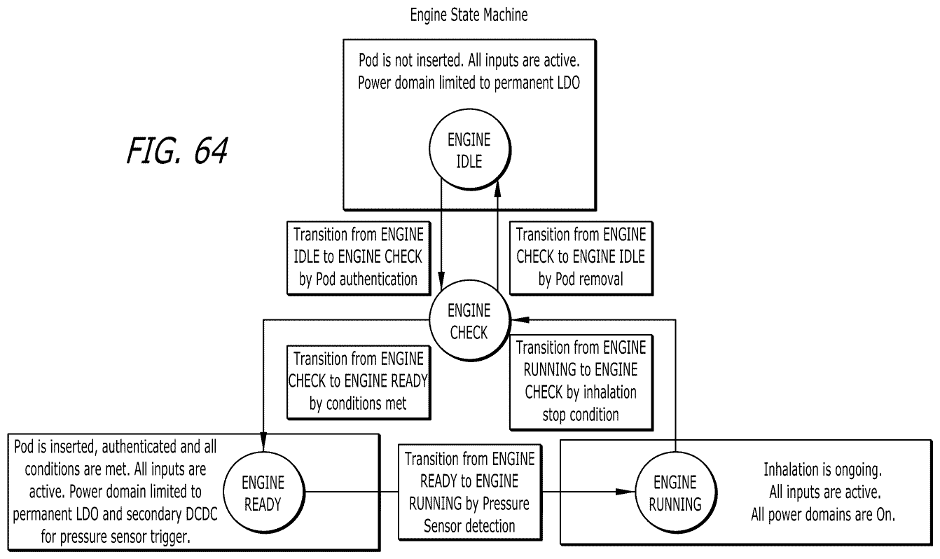

Referring now to FIG. 64 of the accompanying drawings, the driver 210 of some embodiments incorporates an engine state machine. The engine state machine is preferably implemented in executable instructions stored in the memory and executed by the processor of the controller 303. The engine state machine is configured to control the operation of the driver 210 when the driver 210 is used with the pod 110. The engine state machine transitions between different states to change the mode of operation of the driver 210. The changes in mode optimise the operation of the driver 210 and ensure robust security and operation.

The driver 210 is in the ENGINE IDLE state when the pod 110 is not inserted in the cavity 110. In this state, all inputs to the driver 210 may be active. The driver 210 limits the power domain to the low drop out regulator 304 so that the controller 303 remains powered while other power domains are not in use. This minimises unnecessary power consumption (e.g. by the load driver circuits) during the idle state.

When the pod 110 is inserted into the cavity 211, the driver 210 transitions from the ENGINE IDLE state to the ENGINE CHECK state. In the ENGINE CHECK state, the driver 210 authenticates the pod 110. If all conditions are met (including pod authentication success), then the driver 210 transitions to the ENGINE READY state. Otherwise, if all conditions are not met or if the pod 110 is removed, the driver 210 transitions back to the ENGINE IDLE state.

Once the driver 210 enters the ENGINE READY state, the controller 303 controls the power domains within the driver 210 to activate the first DC-DC converter circuit 373 to enable the air flow sensor 251 to detect a change in pressure indicative of inhalation. The driver 210 is thus ready to be activated to generate a mist within the pod 110 when a user draws on the mouthpiece.

In the event that the air flow sensor 251 detects a change in pressure indicative of inhalation, the driver 210 transitions to the ENGINE RUNNING state. When the driver 210 is in the ENGINE RUNNING state, the controller 303 activates all power domains or the majority of the power domains within the driver 210, including the secondary DC-DC converter 305. When the driver 210 is in the ENGINE RUNNING state, the driver 210 outputs a drive signal to the pod 110 to cause the pod 110 to generate a mist for inhalation by a user.

In the event that the air flow sensor 251 detects a further change in air pressure indicative of inhalation by a user stopping, the driver 210 transitions to the ENGINE CHECK state.

The driver 210 thus transitions between the various states of the engine state machine to control operation of the driver 210 when the driver 210 is used with the pod 110 and based on inhalation by a user.

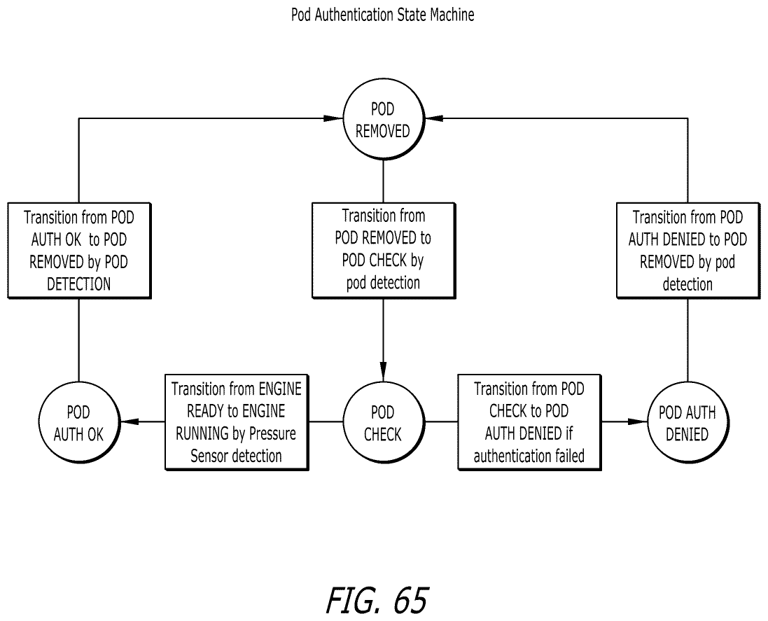

Referring now to FIG. 65 of the accompanying drawings, the driver 210 of some embodiments incorporates a pod authentication state machine which may be implemented in executable instructions stored in the memory and executed by the processor of the controller 303. The authentication state machine controls the mode of operation of the driver 210 based on the authentication status of the pod 110. The authentication states ensure robust checking of the pod 211 and minimise the risk of a counterfeit or unauthorised pod from being used with the driver 210.

The driver 210 enters a POD REMOVED state when the pod 110 is not received within the cavity 211. The driver 210 transitions to a POD CHECK state when the pod 110 is inserted into the cavity 211.

In the POD CHECK state, the driver 210 performs the authentication sequence to authenticate the pod 110. If the pod 110 is authenticated successfully, then the driver 210 transitions to the POD AUTH OK state and the driver 210 is allowed to activate the pod 110 to generate a mist. If, on the other hand, the POD CHECK state identifies that authentication of the pod 110 fails, then the driver 210 enters the POD AUTH DENIED state and activation of the pod 110 by the driver 210 is prevented.

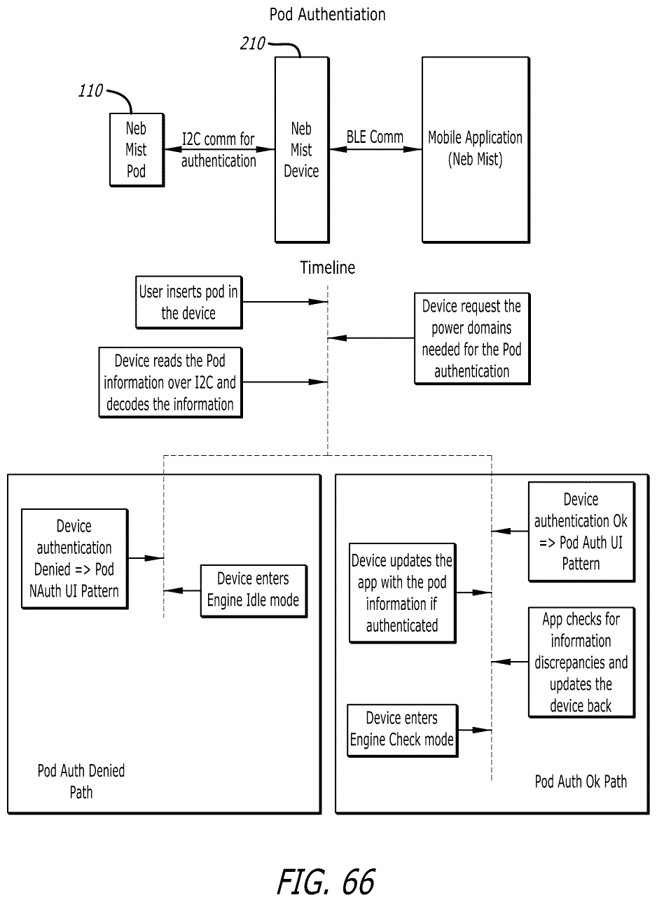

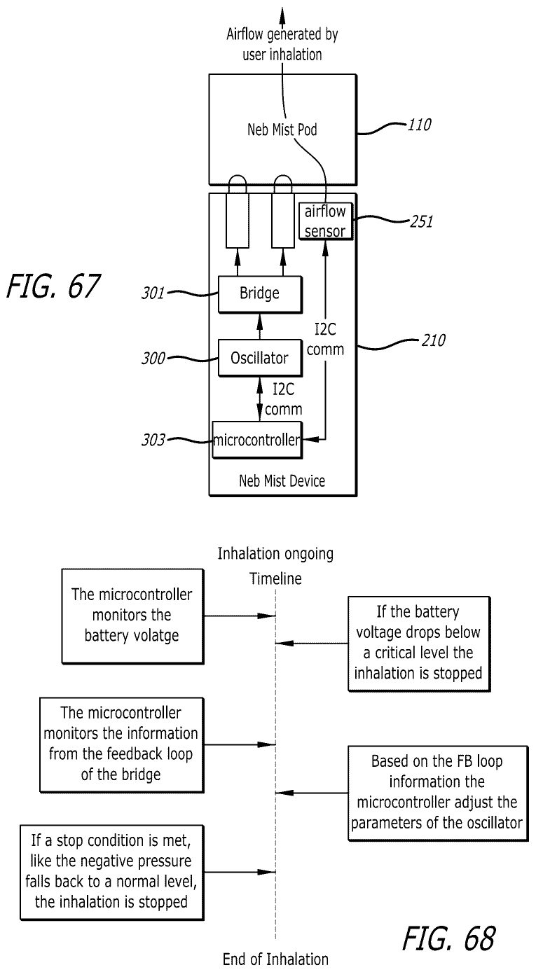

Referring now to FIG. 66 of the accompanying drawings, the authentication timeline that is followed by the driver 210 to authenticate the pod 110 is shown to indicate the timeline followed by the driver 210 as the driver 210 transitions between the states of the pod authentication state machine. As shown in FIG. 66, the driver 210 may communicate with the pod 110 via the I2C bus. The driver 210 may also communicate with a mobile application running on a separate computing device, such as a smartphone. The communication with the computing device may be via Bluetooth or BLE. The communication with the computing device enables the mobile application to monitor and control the driver 210 and record data in relation to the use of the driver 210 and/or the pod 110.

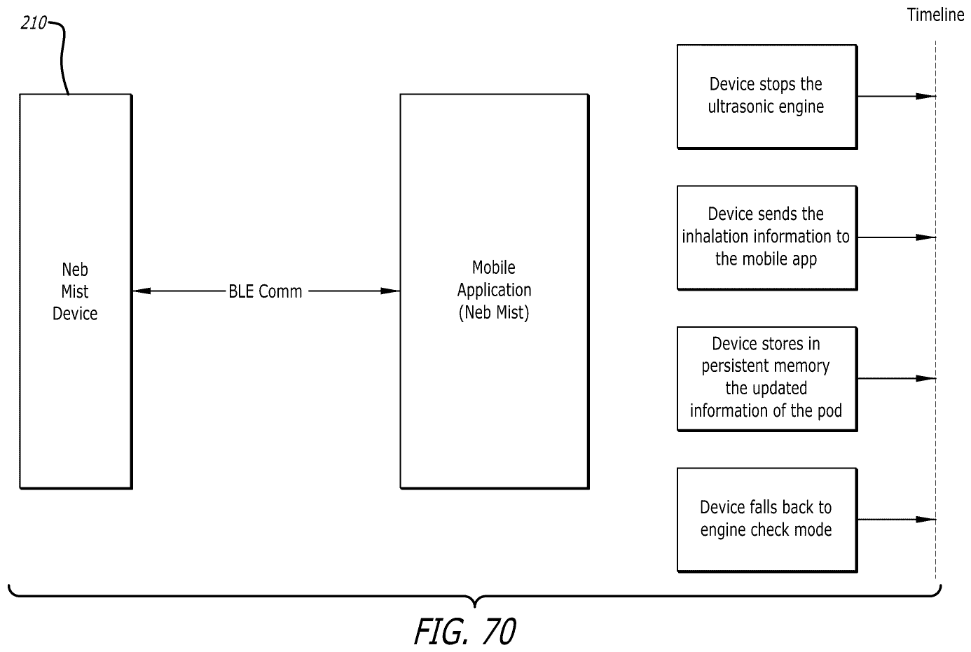

Referring now to FIGS. 68-70 of the accompanying drawings, when the pod 110 is attached to the driver 210, the air flow sensor 251 senses a change of air pressure which is indicative of inhalation by a user and the controller 303 controls the operation of the PMIC (oscillator) 300. The PMIC 300 outputs a signal to the bridge IC 301 which generates an AC drive signal to drive the pod 110 to generate a mist.

With reference to FIG. 68, as inhalation is ongoing, the controller 303 monitors the voltage of the battery 228. If the battery voltage drops below a threshold or critical level, the controller 303 stops the operation of the driver 210.

The controller 303 monitors information from the feedback loop of the bridge IC 301 during inhalation. Based on the feedback loop from the bridge IC 301, the controller 303 controls the PMIC 300 to adjust the parameters of the oscillator configuration within the PMIC 300. For example, the controller 303 may control the PMIC 300 to vary the frequency of the driver signal which drives the pod 110.

If a stop condition is met, such as a change of air pressure indicative of inhalation stopping, then the driver 210 returns to the ENGINE READY state.

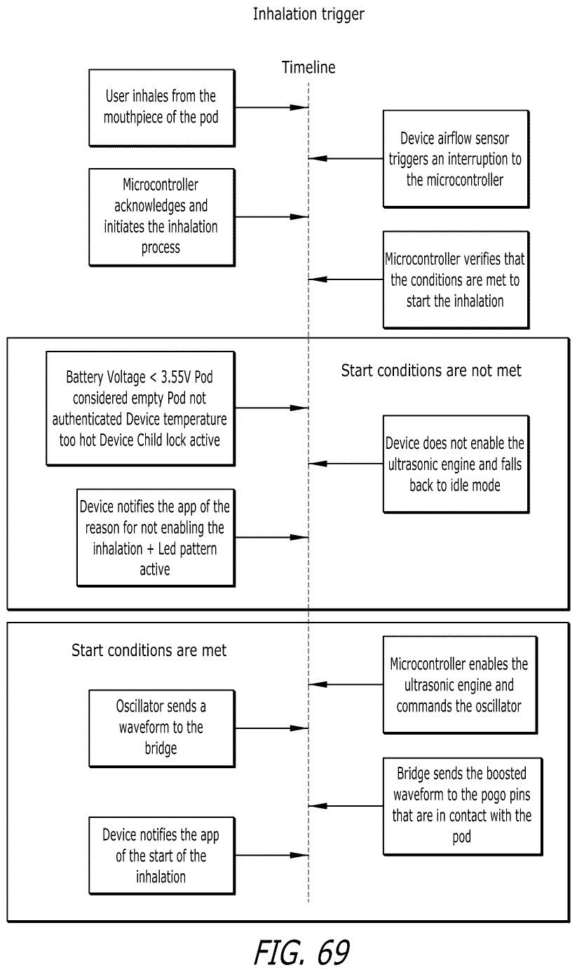

Referring to FIG. 69 of the accompanying drawings, start conditions for activating the driver 210 to control the pod 110 to generate a mist may not be met in the event that certain conditions are not met. For instance, the voltage of the battery 228 being below a threshold, such as below 3.55V. Other conditions include the pod 110 being considered to be empty of liquid or the pod not being authenticated successfully by the device 210.

A further condition in which the pod 210 does not activate the pod 110 is if the driver 210 is at a temperature above or below a safety threshold, for instance if the device driver 210 is too hot. The driver 210 may also not activate the pod 110 to generate a mist in the event that a child lock implemented in the driver 210 is activated. The child lock may be used to prevent a user that is below a certain age from using the device. The child lock is preferably implemented in the driver 210 as electronic functionality that may be encrypted or otherwise protected to ensure that only a verified user over a certain age is able to activate and use the device.

In some examples, the driver 210 communicates with a mobile application on a computing device to verify the age of a user before the driver 210 removes the child block. In these examples, the mobile application may interface with external, regulatory, official or government systems to verify the age and/or identify of a user. The mobile application may use a camera or biometric sensors on the computing device to identify a user. In other examples, the child lock is operational within the driver 210 to verify the identity or age of a user directly from the driver 210 (e.g. using a fingerprint or other biometric sensor provide on the driver 210).

In the event that the driver 210 identifies that the start conditions are not met, the driver 210 notifies the user of the start conditions not being met by controlling the illumination/sequence of the LEDs 321-323.

In the event that the driver 210 determines that the start conditions are met, the controller 303 controls the driver 210 to activate the pod 110 to generate a mist.

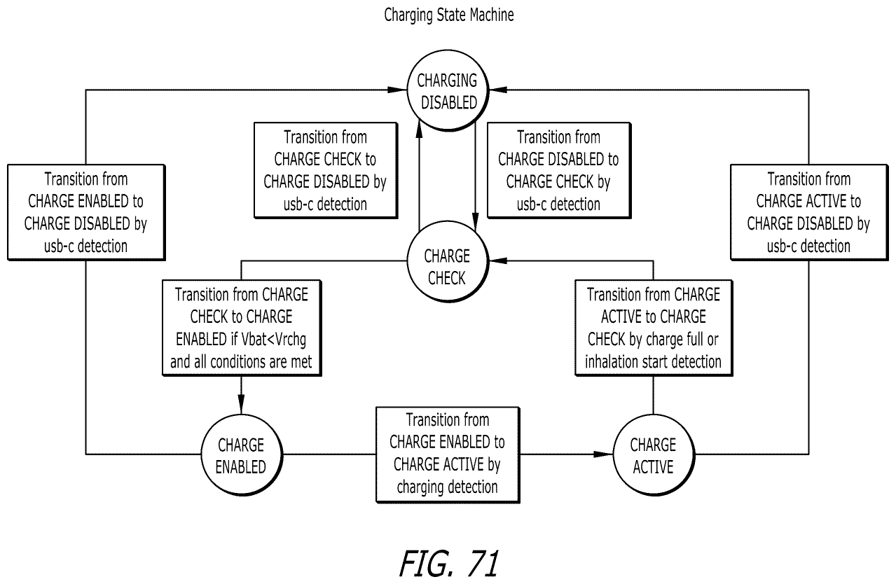

Referring now to FIG. 71 of the accompanying drawings, the driver 210 of some examples incorporate a charging state machine that is preferably implemented in the executable instructions stored in the memory. The executable instructions are executed by the processor of the controller 303 to control the operation of the driver 210 and transition the driver 210 between the various states when the driver 210 is being charged or not charged by an external power source.

The driver 210 enters a CHARGING DISABLED state. In the event that the driver 210 detects that an external power supply is connected to the USB socket 306, the driver 210 enters the CHARGE CHECK state. In this state, if the driver 210 determines that the voltage of the battery 228 is less than the voltage of the power supply connected to the USB socket 306, the driver 210 enters a CHARGE ENABLED state. The driver 210 detects that the external power source is disconnected from the USB port 306, the driver 210 returns to the CHARGING DISABLED state.

The driver 210 transitions from the CHARGE ENABLED state to a CHARGE ACTIVE state. In the event that the driver 210 detects that the charge level of the battery 228 is full or if the driver 210 detects the start of inhalation, the driver 210 transitions to the CHARGE CHECK state. Again, if an external power source is disconnected from the USB port 306, the driver 210 transitions from the CHARGE ACTIVE state to the CHARGING DISABLED state.

The charging state machine therefore provides a robust process for reliably charging the battery 228 in the driver 210. The charging state machine improves safety by minimising the risk of excess current being drawn from an external power source by disabling charging (via the high side switches described herein) during inhalation.

The foregoing outlines features of several examples or embodiments so that those of ordinary skill in the art may better understand various aspects of the present disclosure. Those of ordinary skill in the art should appreciate that they may readily use the present disclosure as a basis for designing or modifying other processes and structures for carrying out the same purposes and/or achieving the same advantages of various examples or embodiments introduced herein. Those of ordinary skill in the art should also realise that such equivalent constructions do not depart from the spirit and scope of the present disclosure, and that they may make various changes, substitutions, and alterations herein without departing from the spirit and scope of the present disclosure.

Although the subject matter has been described in language specific to structural features or methodological acts, it is to be understood that the subject matter of the appended claims is not necessarily limited to the specific features or acts described above. Rather, the specific features and acts described above are disclosed as example forms of implementing at least some of the claims.

Various operations of examples or embodiments are provided herein. The order in which some or all of the operations are described should not be construed to imply that these operations are necessarily order dependent. Alternative ordering will be appreciated having the benefit of this description. Further, it will be understood that not all operations are necessarily present in each embodiment provided herein. Also, it will be understood that not all operations are necessary in some examples or embodiments.

Moreover, “exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application and the appended claims are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, or variants thereof are used, such terms are intended to be inclusive in a manner similar to the term “comprising”. Also, unless specified otherwise, “first,” “second,” or the like are not intended to imply a temporal aspect, a spatial aspect, an ordering, etc. Rather, such terms are merely used as identifiers, names, etc. for features, elements, items, etc. For example, a first element and a second element generally correspond to element A and element B or two different or two identical elements or the same element.

Also, although the disclosure has been shown and described with respect to one or more implementations, equivalent alterations and modifications will occur to others of ordinary skill in the art based upon a reading and understanding of this specification and the annexed drawings. The disclosure comprises all such modifications and alterations and is limited only by the scope of the following claims. In particular regard to the various functions performed by the above described features (e.g., elements, resources, etc.), the terms used to describe such features are intended to correspond, unless otherwise indicated, to any features which performs the specified function of the described features (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

Examples or embodiments of the subject matter and the functional operations described herein can be implemented in digital electronic circuitry, or in computer software, firmware, or hardware, including the structures disclosed in this specification and their structural equivalents, or in combinations of one or more of them.

Some examples or embodiments are implemented using one or more modules of computer program instructions encoded on a computer-readable medium for execution by, or to control the operation of, a data processing apparatus. The computer-readable medium can be a manufactured product, such as hard drive in a computer system or an embedded system. The computer-readable medium can be acquired separately and later encoded with the one or more modules of computer program instructions, such as by delivery of the one or more modules of computer program instructions over a wired or wireless network. The computer-readable medium can be a machine-readable storage device, a machine-readable storage substrate, a memory device, or a combination of one or more of them.

The terms “computing device” and “data processing apparatus” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment, or a combination of one or more of them. In addition, the apparatus can employ various different computing model infrastructures, such as web services, distributed computing and grid computing infrastructures.

The processes and logic flows described in this specification can be performed by one or more programmable processors executing one or more computer programs to perform functions by operating on input data and generating output.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices.

When used in this specification and the appended claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A mist inhaler including a pod and a driver for delivering a mist including nicotine, the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid chamber containing a liquid to be atomised, the liquid comprising the nicotine;
a spacer positioned within the housing between the liquid chamber and the second end wall, the spacer including a hollow interior surrounded by a perimeter;
a fluid flow manifold positioned at least partially within the hollow interior of the spacer and including a first side proximate the first end wall, and a second side proximate the second end wall, the first side of the fluid flow manifold including a channel having a first portion and a second portion, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, and through the fluid flow manifold to the sonication chamber, a first end of the mist outlet conduit being in fluid communication with a mist outlet port in the first end wall of the housing and a second end of the mist outlet conduit being in fluid communication with the sonication chamber, and
the driver comprising:
a driver casing having a base end, an open end, and an air intake aperture positioned between the base end and the open end, the open end incorporating a casing recess;
electronic components configured to generate control signals to control the operation of the pod, and drive signals to drive the ultrasonic transducer to atomise the liquid to generate the mist; and
a bulkhead positioned within the casing recess, the bulkhead including:
a body substantially of a resiliently deformable material, the body having a top surface, a bottom surface and a portion that extends from the top surface to form a seal;
a plurality of apertures formed in the body, each aperture extending through the top surface and the bottom surface, the plurality of apertures being positioned in an area at least partly surrounded by the seal; and
a bulkhead intake conduit in airtight fluid communication with the air intake aperture and with a first aperture of the plurality of apertures, the bulkhead intake conduit being formed entirely within the driver and forming a part of an intake air flow path, the bulkhead intake conduit being configured to conduct air from the air intake aperture to the pod through the first aperture of the plurality of apertures in the bulkhead,
a part of the pod being received into the casing recess, the seal of the driver contacting the part of the pod and forming a substantially airtight seal therebetween to prevent air leaking out from between the bulkhead and the part of the pod, and
an intake air flow path extending from the air intake aperture in the driver casing to the sonication chamber, the intake air flow path passing through the bulkhead intake conduit in the driver, and the air intake conduit in the pod.

2. The mist inhaler of claim 1, wherein the first portion of the channel in the fluid flow manifold extends to the second portion tangentially.

3. The mist inhaler of claim 1, wherein the seal is an upturned lip extending around the periphery of the top surface.

4. The mist inhaler of claim 1, wherein the seal spaces the part of the pod from the top surface of the body of the bulkhead by a distance, the distance defining a chamber therebetween.

5. The mist inhaler of claim 4, wherein the chamber defines a part of the intake air flow path.

6. The mist inhaler of claim 1, further including a plurality of electrical connectors, each of the electrical connectors extending through a respective one of the plurality of apertures formed in the body of the bulkhead, the body of the bulkhead providing an airtight seal around each of the electrical connectors such that air cannot flow from the top surface of the body to the bottom surface of the body.

7. The mist inhaler of claim 1, further including:
an air flow sensor for detecting a change in air pressure relative to an ambient air pressure and providing an air flow pressure signal to the electronic components within the driver;
an air flow sensor holder within the driver casing, the air flow sensor holder including an opening for accepting the air flow sensor, the opening having a perimeter contacting the air flow sensor to form an airtight seal with the air flow sensor to create an ambient air space and an intake air space separated by the air flow sensor, wherein the air flow sensor holder includes:
a channel for supplying air at substantially the ambient air pressure to the ambient air space; and
a further channel in fluid communication with the intake air flow path extending from the driver casing to the pod, the further channel for detecting an air pressure in the intake air flow path lower than the ambient air pressure, the lower air pressure being indicative of a user using the pod.

8. The mist inhaler of claim 7, further comprising at least one LED, the air flow sensor holder including a recess for accepting a respective one of the at least one LEDs.

9. The mist inhaler of claim 8, wherein the air flow sensor holder comprises:
a body portion which includes the opening for accepting the air flow sensor, the channel for supplying air at substantially the ambient air pressure to the ambient air space, and the further channel for detecting an air pressure in the intake air flow path lower than the ambient air pressure; and
an auxiliary portion extending from the base portion, the auxiliary portion covering each of the at least one LEDs and including a first side and a second side,
the second side of the auxiliary portion including the recess for accepting a respective one of the at least one LEDs,
the auxiliary portion further including at least one projection extending from and substantially normal to the first side of the auxiliary portion.

10. The mist inhaler of claim 9, wherein the projection is translucent such that, when the at least one LED is activated, light is visible through the projection.

11. The mist inhaler of claim 8, wherein the at least one LED is a plurality of LEDs, and wherein a corresponding projection is provided for each LED.

12. A mist inhaler including a pod and a driver for delivering a mist including nicotine, the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid chamber, containing a liquid to be atomised, the liquid comprising the nicotine;
a spacer positioned within the housing between the liquid chamber and the second end wall, the spacer including a hollow interior surrounded by a perimeter;
a fluid flow manifold positioned at least partially within the hollow interior of the spacer and including a first side proximate the first end wall, and a second side proximate the second end wall, the first side of the fluid flow manifold including a channel having a first portion and a second portion, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, and through the fluid flow manifold to the sonication chamber, a first end of the mist outlet conduit being in fluid communication with a mist outlet port in the first end wall of the housing and a second end of the mist outlet conduit being in fluid communication with the sonication chamber, and
the driver comprising:
a substrate;
an air flow sensor coupled to the substrate for detecting a change in air pressure relative to the ambient air pressure;
at least one LED coupled to the substrate; and
an air flow sensor holder coupled to the substrate, the air flow sensor holder including:
a body portion having an opening for accepting the air flow sensor, the opening having a perimeter contacting the air flow sensor to form an airtight seal with the air flow sensor to create an ambient air space and an intake air space separated by the air flow sensor, wherein the body portion of the air flow sensor holder includes:
a channel for supplying air at substantially ambient air pressure to the ambient air space;
a further channel in fluid communication with an intake air flow path extending from the driver casing to the pod, the further channel for detecting an air pressure in the intake air flow path lower than the ambient air pressure, the lower air pressure being indicative of a user using the pod;
the air flow sensor holder having a recess for accepting a respective one of the at least one LEDs,
an intake air flow path extending from the air intake aperture in the driver casing to the sonication chamber, the intake air flow path passing through the bulkhead intake conduit in the driver, and the air intake conduit in the pod.

13. The driver of claim 12, wherein the air flow sensor holder further comprises an auxiliary portion extending from the base portion, the auxiliary portion including a first side and a second side, the auxiliary portion covering each of the at least one LEDs and the second side of the auxiliary portion including the recess for accepting a respective one of the at least one LEDs, the auxiliary portion further including at least one projection extending from and substantially normal to the first side of the auxiliary portion.

14. The driver of claim 13, wherein the projection is translucent such that, when the at least one LED is activated, light is visible through the projection.

15. The driver of claim 12, wherein the substrate is a PCB.

16. The driver of claim 12, wherein the driver includes a main PCB mounted therein, the substrate being the main PCB.

17. The driver of claim 12, wherein the air flow sensor holder is formed of a single piece.

18. The driver of claim 12, wherein the air flow sensor holder is of a resiliently deformable material.

19. The driver of claim 18, wherein the air flow sensor holder is of Shore A 40 silicone.

20. The driver of claim 12, wherein the at least one LED is a plurality of LEDs, and wherein a corresponding projection is provided for each LED.
